(19) United States
(12) Patent Application Publication
(10) Pub. No.: US 2003/0092042 A1
(43) Pub. Date: May 15, 2003

(54) Amplified oncogenes and their involvement in cancer

(76) Inventors: David Mu, Jericho, NY (US); Scott Powers, Greenlawn, NY (US)

Correspondence Address: HELLER EHRMAN WHITE & MCAULIFFE LLP, 1666 K STREET,NW, SUITE 300, WASHINGTON, DC, 20006, US

(21) Appl. No.: 10/228,264

(22) Filed: Aug. 27, 2002

Related U.S. Application Data
(60) Provisional application No. 60/314,655, filed on Aug. 27, 2001.
(60) Provisional application No. 60/330,797, filed on Oct. 31, 2001.
(60) Provisional application No. 60/340,863, filed on Dec. 19, 2001.
(60) Provisional application No. 60/375,027, filed on Apr. 25, 2002.

(51) Int. Cl. A61K 31/00; C12Q 1/68; G06F 19/00; G01N 33/48; G01N 33/50; A61K 48/00

(52) U.S. Cl. 435/6; 702/20; 514/1; 514/44

(57) ABSTRACT

There are disclosed methods and compositions for the diagnosis, prevention, and treatment of tumors and cancers in mammals such as humans, utilizing the Galanin, Galanin Receptor 2 (GALR2) and Galanin Receptor 3 (GALR3) genes, which are amplified in breast and/or lung and/or brain and/or colon and/or prostate and/or ovarian cancer genes. The Galanin, GALR2 and GALR3 genes and their expressed protein products and antibodies are used diagnostically or as targets for cancer therapy or vaccine; they are also used to identify compounds and reagents useful in cancer diagnosis, prevention, and therapy. There is also disclosed a GALR2-GALR3 heterocomplex formation during tumorigenesis.

This application claims priority to U.S. Serial No. 60/314,655, filed Aug. 27, 2001; U.S. Serial No. 60/330,797, filed Oct. 31, 2001; U.S. Serial No. 60/340,863, filed Dec. 19, 2001; and U.S. Serial No. 60/375,027, filed Apr. 25, 2002. The entireties of all above applications are hereby incorporated by reference.

### BACKGROUND OF THE INVENTION

1. Field of the Invention

The present invention relates to oncogenes and to cancer diagnostics and therapeutics. More specifically, the present invention relates to amplified and overexpressed Galanin, Galanin Receptor 2 (GALR2) and Galanin Receptor 3 (GALR3) genes that are involved in certain types of cancers. The invention also relates to a functionally important GALR2-GALR3 heterocomplex formation during tumorigenesis. The invention pertains to the amplified genes, their encoded proteins, and antibodies, inhibitors, activators and the like and their use in cancer diagnostics, vaccines, and anti-cancer therapy, including breast, lung, brain, colon, prostate, and ovarian cancers.

2. Background of the Invention

Cancer and Gene Amplification:

Cancer is the second leading cause of death in the United States, after heart disease (Boring, et al., CA Cancer J. Clin., 43:7, 1993), and it develops in one in three Americans. One of every four Americans dies of cancer. Cancer features uncontrolled cellular growth, which results either in local invasion of normal tissue or systemic spread of the abnormal growth. A particular type of cancer or a particular stage of cancer development may involve both elements.

The division or growth of cells in various tissues functioning in a living body normally takes place in an orderly and controlled manner. This is enabled by a delicate growth control mechanism, which involves, among other things, contact, signaling, and other communication between neighboring cells. Growth signals, stimulatory or inhibitory, are routinely exchanged between cells in a functioning tissue. Cells normally do not divide in the absence of stimulatory signals, and will cease dividing when dominated by inhibitory signals. However, such signaling or communication becomes defective or completely breaks down in cancer cells. As a result, the cells continue to divide; they invade adjacent structures, break away from the original tumor mass, and establish new growth in other parts of the body. The latter progression to malignancy is referred to as “metastasis.”

Cancer generally refers to malignant tumors, rather than benign tumors. Benign tumor cells are similar to normal, surrounding cells. These types of tumors are almost always encapsulated in a fibrous capsule and do not have the potential to metastasize to other parts of the body. These tumors affect local organs but do not destroy them; they usually remain small without producing symptoms for many years. Treatment becomes necessary only when the tumors grow large enough to interfere with other organs. Malignant tumors, by contrast, grow faster than benign tumors; they penetrate and destroy local tissues. Some malignant tumors may spread throughout the body via blood or the lymphatic system. The unpredictable and uncontrolled growth makes malignant cancers dangerous, and fatal in many cases. These tumors are not morphologically typical of the original tissue and are not encapsulated. Malignant tumors commonly recur after surgical removal.

Accordingly, treatment ordinarily targets malignant cancers or malignant tumors. The intervention of malignant growth is most effective at the early stage of the cancer development. It is thus exceedingly important to discover sensitive markers for early signs of cancer formation and to identify potent growth suppression agents associated therewith. The development of such diagnostic and treatment agents involves an understanding of the genetic control mechanisms for cell division and differentiation, particularly in connection with tumorigenesis. Cancer is caused by inherited or acquired mutations in cancer genes, which have normal cellular functions and which induce or otherwise contribute to cancer once mutated or expressed at an abnormal level. Certain well-studied tumors carry several different independently mutated genes, including activated oncogenes and inactivated tumor suppressor genes. Each of these mutations appears to be responsible for imparting some of the traits that, in aggregate, represent the full neoplastic phenotype (Land et al., Science, 222:771, 1983; Ruley, Nature, 4:602, 1983; Hunter, Cell, 64:249, 1991).

One such mutation is gene amplification. Gene amplification involves a chromosomal region bearing specific genes undergoing a relative increase in DNA copy number, thereby increasing the copies of any genes that are present. In general, gene amplification results in increased levels of transcription and translation, producing higher amounts of the corresponding gene mRNA and protein. Amplification of genes causes deleterious effects, which contribute to cancer formation and proliferation (Lengauer et al. Nature, 396:643-649,1999).

It is commonly appreciated by cancer researchers that whole collections of genes are demonstrably overexpressed or differentially expressed in a variety of different types of tumor cells. Yet, only a very small number of these overexpressed genes are likely to be causally involved in the cancer phenotype. The remaining overexpressed genes likely are secondary consequences of more basic primary events, for example, overexpression of a cluster of genes, involved in DNA replication. On the other hand, gene amplification is established as an important genetic alteration in solid tumors (Knuutila et al., Am. J. Pathol., 152(5):1107-23, 1998; Knuutila et al., Cancer Genet. Cytogenet., 100(1):25-30, 1998).

The overexpression of certain well known genes, for example, c-myc, has been observed at fairly high levels in the absence of gene amplification (Yoshimoto et al., JPN J. Cancer Res., 77(6):540-5, 1986), although these genes are frequently amplified (Knuutila et al., Am. J. Pathol., 152(5):1107-23, 1998) and thereby activated. Such a characteristic is considered a hallmark of oncogenes. Overexpression in the absence of amplification may be caused by higher transcription efficiency in those situations. In the case of c-myc, for example, Yoshimoto et al. showed that its transcriptional rate was greatly increased in the tested tumor cell lines. The characteristics and interplay of overexpression and amplification of a gene in cancer tissues, therefore, provide significant indications of the gene's role in cancer development. That is, increased DNA copies of certain genes in tumors, along with and beyond its overexpression, may point to their functions in tumor formation and progression.

It must be remembered that overexpression and amplification are not the same phenomenon. Overexpression can be obtained from a single, unamplified gene, and an amplified gene does not always lead to greater expression levels of mRNA and protein. Thus, it is not possible to predict whether one phenomenon will result in or related to the other. However, in situations where both amplification of a gene and overexpression of the gene product occur in cells or tissues that are in a precancerous or cancerous state, then that gene and its product present both a diagnostic target and a therapeutic opportunity for intervention. Because some genes are sometimes amplified as a consequence of their location next to a true oncogene, it is also beneficial to determine the DNA copy number of nearby genes in a panel of tumors so that amplified genes that are in the epicenter of the amplification unit can be distinguished from amplified genes that are occasionally amplified due to their proximity to another, more relevant amplified gene.

Thus, discovery and characterization of amplified cancer genes, along with and in addition to their features of overexpression or differential expression, will be a promising avenue that leads to novel targets for diagnostic, vaccines, and therapeutic applications. Additionally, the completion of the working drafts of the human genome and the paralleled advances in genomics technologies offer new promises in the identification of effective cancer markers and the anti-cancer agents. The high-throughput microarray detection and screening technology, computer-empowered genetics and genomics analysis tools, and multi-platform functional genomics and proteomics validation systems, all assist in applications in cancer research and findings. With the advent of modern sequencing technologies and genomic analyses, many unknown genes and genes with unknown or partially known functions can be revealed.

Galanin and Galanin Receptors:

Until the recent invention, Galanin, GALR2 and GALR3 have been thought to be associated with cancer, but have not been successfully isolated and characterized to have their role understood in tumor development.

Galanin is a 29- or 30-amino acid neuropeptide with a complex role in pain processing. Several galanin receptor subtypes are present in dorsal root ganglia and spinal cord with a differential distribution. The galanin receptor type 1 (GALR1) is known predominantly to be expressed in basal forebrain, hypothalamus, as well as spinal cord. On the other hand, the galanin receptor type 2 (GALR2) has been found to be widely distributed in brain and is also present in the pituitary gland and peripheral tissues (Depczynski et al., Annals of the New York Academy of Sciences, Dec. 21, 1998, 863 pp. 120-128, United States). Recently, GALR2 is found to initiate multiple signaling pathways in small cell lung cancer cells by coupling to G(q), G(i) and G(12) proteins (Wittau et al., Oncogene 19(37):4199-209, (2000)).

Sethi and Rozengurt described GALR3 involvement in cancer (Cancer Res. 51, 1674-1679 (1991)) by disclosing the fact that Galanin stimulates Ca²⁺ mobilization, inositol phosphate accumulation, and clonal growth in small cell lung cancer cells. Addition of the neuropeptide galanin to small cell lung cancer (SCLC) cells loaded with the fluorescent Ca²⁺ indicator fura-2-tetraacetoxymethylester causes a rapid and transient increase in the intracellular concentration. Galanin increases Ca²⁺ ([Ca²⁺]i) followed by homologous desensitization. Galanin increases [Ca²⁺]i in a concentration-dependent fashion with half-maximum effect (EC₅₀) at 20-22 nM in H69 and H510 SCLC cells. Galanin mobilizes Ca²⁺ from intracellular stores since its effects on [Ca²⁺]i are not blocked by chelation of extracellular Ca²⁺. Pretreatment with pertussis toxin (200 ng/ml for 4 h) does not prevent galanin-induced Ca²⁺mobilization. In contrast, direct activation of protein kinase C with phorbol esters attenuates the Ca²⁺response induced by galanin. The effects of galanin can be dissociated from changes in membrane potential: galanin does not increase membrane potential in SCLC cells loaded with bis (1,3-diethylthiobarbiturate)-trimethineoxonol and induces Ca²⁺mobilization in depolarized SCLC cells, i.e., in cells suspended in a solution containing 145 mM K+instead of Na+. Galanin also causes an increase in the formation of inositol phosphates in a time- and dose-dependent manner (EC₅₀ 10 nM). A rapid increase in the inositol trisphosphate fraction is followed by a slower increase in the inositol monophosphate fraction. Galanin stimulates clonal growth of both H69 and H510 cells in semisolid (agarose-containing) medium. This growth-promoting effect is sharply dependent on galanin concentration (EC₅₀ 20 nM) and markedly inhibited by [Arg6, D-Trp7, 9, Mephe 8] substance P, a recently identified broad-spectrum neuropeptide antagonist. The results show that galanin receptors are coupled to inositol phosphate and [Ca²⁺]i responses in SCLC cells and, in particular, that this neuropeptide can act as a direct growth factor for these human cancer cells.

### SUMMARY OF THE INVENTION

The present invention relates to isolation, characterization, overexpression and implication of genes, including amplified genes, in cancers, methods and compositions for use in diagnosis, vaccines, prevention, and treatment of tumors and cancers, for example, breast cancer, lung cancer, brain cancer, colon cancer, prostate cancer, and ovarian cancer, in mammals, for example, humans. The invention is based on the finding of novel traits of Galanin, Galanin Receptor 2 (GALR2) and Galanin Receptor 3 (GALR3) genes. The invention also relates to a functionally important heterocomplex of GALR2-GALR3 that is involved in tumorigenesis.

Galanin, the ligand to the galanin receptors, is frequently amplified and overexpressed in the lung tumors containing amplified and overexpressed galanin receptors. This suggests that the galanin autocrine loop is under genetic pressure in lung cancers to be over-represented, indicating an important role for galanin and its receptors in lung cancers.

As demonstrated herein, Galanin appears to be at the epicenter of an amplicon that is amplified in over 70% of human lung tumors, indicating that the gene has an important biological function so that increased DNA copies of the gene are selected for during tumor formation. As demonstrated herein, the Galanin gene also is frequently amplified and overexpressed in human lung cancers. As disclosed herein, the Galanin gene and its expressed protein product can thus be used diagnostically or as targets for cancer therapy; and they can also be used to identify and design compounds useful in the diagnosis, prevention, and therapy of tumors and cancers (for example, lung cancer).

GALR2 is coamplified with GALR3 frequently in the same human lung, breast, colon, prostate, and ovarian primary tumors, implying that both GALR2 and GALR3 are selected for during tumorigenesis and that a functionally important heterocomplex of GALR2-GALR3 appears to exist. GALR2 and GALR1 share 58% and 36% amino acid identity with GALR3, respectively, and it is found that only GALR2 is amplified in the same lung tumors that also contain increased GALR3 gene copy number.

GALR3 is involved in cancer as Galanin stimulates Ca²⁺ mobilization, inositol phosphate accumulation, and clonal growth in small cell lung cancer cells. As disclosed for the first time herein, GALR3 appears to be at the epicenter of an amplicon that is amplified in 50% of human lung, breast, colon, prostate, and ovarian tumors, implying that the gene has an important biological function so that increased DNA copies of the gene are selected for during tumor formation. The GALR3 gene also is frequently overexpressed in human lung cancers. The GALR3 gene and its expressed protein product can thus be used diagnostically or as targets for cancer therapy; and they can also be used to identify and design compounds useful in the diagnosis, prevention, and therapy of tumors and cancers (for example, lung, breast, colon, prostate, and ovarian cancers).

According to one aspect of the present invention, the use of Galanin, GALR2 and/or GALR3 in gene therapy, development of antisense nucleic acids and small interfering RNAs (siRNAs), and development of immunodiagnostics or immunotherapy are provided. The present invention also includes production and the use of antibodies, for example, monoclonal, polyclonal, single-chain and engineered antibodies (including humanized antibodies) and fragments, which specifically bind Galanin protein, GALR2 protein, GALR3 protein, or GALR2-GALR3 heterocomplex. The invention also features antagonists and inhibitors of Galanin protein, GALR2 protein, GALR3 protein, or GALR2-GALR3 heterocomplex protein that can inhibit one or more of the functions or activities of Galanin protein, GALR2 protein, GALR3 protein, or GALR2-GALR3 heterocomplex protein. Suitable antagonists can include small molecules (molecular weight below about 500), large molecules (molecular weight above about 500), antibodies, including fragments and single chain antibodies, that bind and interfare or neutralize Galanin protein, GALR2 protein, GALR3 protein, or GALR2-GALR3 heterocomplex protein and which compete with a native form of Galanin protein, GALR2 protein, GALR3 protein, or GALR2-GALR3 heterocomplex protein for binding to a protein that naturally interacts with Galanin protein, GALR2 protein, GALR3 protein, or GALR2-GALR3 heterocomplex protein for the latter's function, and nucleic acid molecules that interfere with transcription of the Galanin, GALR2, or GALR3 genes (for example, antisense nucleic acid molecules, ribozymes and small interfering RNAs (siRNAs)). Useful agonists, ones that may induce certain mutants of Galanin, GALR2, or GALR3 thereby attenuating activities of Galanin, GALR2, or GALR3, also include small and large molecules, and antibodies other than “neutralizing” antibodies.

In addition, the present invention provides an inhibitor of Galanin, GALR2, or GALR3 activity, wherein the inhibitor is an antibody that blocks the oncogenic function or anti-apoptotic activity of Galanin, GALR2, GALR3, or GALR2-GALR3 heterocomplex.

The present invention also provides an inhibitor of Galanin, GALR2, or GALR3 activity, wherein the inhibitor is an antibody that binds to a cell overexpressing Galanin, GALR2, or GALR3 protein, thereby resulting in suppression or death of the cell.

The present invention further features molecules that can decrease the expression of Galanin, GALR2, or GALR3 by affecting transcription or translation. Small molecules (molecular weight below about 500), large molecules (molecular weight above about 500), and nucleic acid molecules, for example, ribozymes, siRNAs and antisense molecules, including antisense RNA, antisense DNA or DNA decoy or decoy molecules (for example, Morishita et al., Ann. N Y Acad. Sci., 947:294-301, 2001; Andratschke et al., Anticancer Res., 21:(5)3541-3550, 2001), may all be utilized to inhibit the expression or amplification.

As mentioned above, the Galanin, GALR2, and GALR3 gene sequences also can be employed in an RNA, interference context. The phenomenon of RNA interference is described and discussed in Bass, Nature, 411: 428-29 (2001); Elbashir et al., Nature, 411: 494-98 (2001); and Fire et al., Nature, 391: 806-11 (1998), where methods of making interfering RNA also are discussed.

In one aspect, the present invention provide methods for diagnosing a cancer, for example, a lung cancer, a brain cancer, a breast cancer, a colon cancer, a prostate cancer, or an ovarian cancer, in a mammal, which comprises, for example, obtaining a biological test sample from a region in the tissue that is suspected to be precancerous or cancerous; obtaining a biological control sample from a region in the tissue or other tissues in the mammal that is normal; and detecting in both the biological test sample and the biological control sample the level of Galanin, GALR2, or GALR3 messenger RNA transcripts, wherein a level of the transcripts higher in the biological subject than that in the biological control sample indicates a cancer in the tissue. In another aspect, the biological control sample may be obtained from a different individual or be a normalized value based on baseline values found in a population.

In another aspect, the present invention provide methods for diagnosing a cancer, such as a lung cancer, a brain cancer, a breast cancer, a colon cancer, a prostate cancer, or an ovarian cancer, in a mammal, which comprises, for example, obtaining a biological test sample from a region in the tissue that is suspected to be precancerous or cancerous; and detecting in the biological subject the number of Galanin, GALR2, or GALR3 DNA copies thereby determining whether the Galanin, GALR2, or GALR3 gene is amplified in the biological test subject, wherein amplification of the Galanin, GALR2, or GALR3 gene indicates a cancer in the tissue.

Another aspect of the present invention provide methods for diagnosing a cancer, for example, a lung cancer, a brain cancer, a breast cancer, a colon cancer, a prostate cancer, or an ovarian cancer, in a mammal, which comprises, for example, obtaining a biological test sample from a region in the tissue that is suspected to be precancerous or cancerous, contacting the samples with anti-Galanin, anti-GALR2, or anti-GALR3 antibodies, and detecting in the biological subject the level of Galanin, GALR2, or GALR3 gene expression, respectively, wherein a level of the Galanin, GALR2, or GALR3 gene expression higher in the biological subject than that in the biological control sample indicates a cancer in the tissue. In an alternative aspect the biological control sample may be obtained from a different individual or be a normalized value based on baseline values found in a population.

In yet another aspect, the present invention relates to methods for comparing and compiling data wherein the data is stored in electronic or paper format. Electronic format can be selected from the group consisting of electronic mail, disk, compact disk (CD), digital versatile disk (DVD), memory card, memory chip, ROM or RAM, magnetic optical disk, tape, video, video clip, microfilm, internet, shared network, shared server and the like; wherein data is displayed, transmitted or analyzed via electronic transmission, video display, telecommunication, or by using any of the above stored formats; wherein data is compared and compiled at the site of sampling specimens or at a location where the data is transported following a process as described above.

In still another aspect, the present invention provide methods for preventing, controlling, or suppressing cancer growth in a mammalian organ and tissue, such as in the lung, brain, breast, colon, prostate, or ovary, which comprises administering an inhibitor of Galanin, GALR2, or GALR3 protein, or GALR2-GALR3 heterocomplex to the organ or tissue, thereby inhibiting Galanin, GALR2, or GALR3 protein or GALR2-GALR3 heterocomplex activities. Such inhibitors may be, inter alia, an antibody to Galanin, GALR2, GALR3 protein, or GALR2-GALR3 heterocomplex, or polypeptide portions thereof, an antagonist to Galanin, GALR2, or GALR3 protein, or GALR2-GALR3 heterocomplex, or other small molecules.

In a further aspect, the present invention provide methods for preventing, controlling, or suppressing cancer growth in a mammalian organ and tissue, such as in the lung, brain, breast, colon, prostate, or ovary, which comprises administering to the organ or tissue a nucleotide molecule that is capable of interacting with Galanin, GALR2, or GALR3 DNA or RNA and thereby blocking or interfering the Galanin, GALR2, or GALR3 gene functions, respectively. Such nucleotide molecule can be an antisense nucleotide of the Galanin, GALR2, or GALR3 gene, a ribozyme of Galanin, GALR2 or GALR3 RNA; a small interfering RNA (siRNA) or, it may be capable of forming a triple helix with the Galanin, GALR2, or GALR3 gene.

In still a further aspect, the present invention provide methods for monitoring the efficacy of a therapeutic treatment regimen for treating a cancer, for example, a lung cancer, and brain cancer, a breast cancer, a colon cancer, a prostate cancer, or an ovarian cancer, in a patient, which comprises obtaining a first sample of cancer cells from the patient; administering the treatment regimen to the patient; obtaining a second sample of cancer cells from the patient after a time period; and detecting in both the first and the second samples the level of Galanin, GALR2, or GALR3 messenger RNA transcripts, wherein a level of the transcripts lower in the second sample than that in the first sample indicates that the treatment regimen is effective to the patient.

In another aspect, the present invention provide methods for monitoring the efficacy of a compound to suppress a cancer, for example, a lung cancer, a brain cancer, a breast cancer, a colon cancer, a prostate cancer, or an ovarian cancer, in a patient in a clinical trial, which comprises obtaining a first sample of cancer cells from the patient; administering the treatment regimen to the patient; obtaining the second sample of cancer cells from the patient after a time period; and detecting in both the first and the second samples the level of Galanin, GALR2, or GALR3 messenger RNA transcripts, wherein a level of the transcripts lower in the second sample than that in the first sample indicates that the compound is effective to suppress such a cancer.

Another aspect of the present invention provide methods for monitoring the efficacy of a therapeutic treatment regimen for treating a cancer, for example, a lung cancer, a brain cancer, a breast cancer, a colon cancer, a prostate cancer, or an ovarian cancer, in a patient, which comprises obtaining a first sample of cancer cells from the patient; administering the treatment regimen to the patient; obtaining a second sample of cancer cells from the patient after a time period; and detecting in both the first and the second samples the number of Galanin, GALR2, or GALR3 DNA copies, thereby determining the Galanin, GALR2, or GALR3 gene amplification state in the first and second samples, wherein a lower number of Galanin, GALR2, or GALR3 DNA copies in the second sample than that in the first sample indicates that the treatment regimen is effective because the cells with amplified Galanin, GALR2 and/or GALR3 are being eliminated or suppressed.

In yet another aspect, the present invention provide methods for monitoring the efficacy of a therapeutic treatment regimen for treating a cancer, for example, a lung cancer, a brain cancer, a breast cancer, a colon cancer, a prostate cancer, or an ovarian cancer, in a patient, which comprises obtaining a first sample of cancer cells from the patient; administering the treatment regimen to the patient; obtaining a second sample of cancer cells from the patient after a time period, contacting the samples with anti-Galanin, anti-GALR2, or anti-GALR3 antibodies, and detecting in the level of Galanin, GALR2, or GALR3 gene expression, respectively, in both the first and the second samples. A lower level of the Galanin, GALR2, or-GALR3 gene expression in the second sample than that in the first sample indicates that the treatment regimen is effective to the patient.

In still another aspect, the present invention provide methods for monitoring the efficacy of a compound to suppress a cancer, for example, a lung cancer, a brain cancer, a breast cancer, a colon cancer, a prostate cancer, or an ovarian cancer, in a patient, for example, in a clinical trial, which comprises obtaining a first sample of cancer cells from the patient; administering the treatment regimen to the patient; obtaining a second sample of cancer cells from the patient after a time period; and detecting in both the first and the second samples the number of Galanin, GALR2, or GALR3 DNA copies, thereby determining the Galanin, GALR2, or GALR3 gene amplification state in the first and second samples, wherein a lower number of Galanin, GALR2, or GALR3 DNA copies in the second sample than that in the first sample indicates that the compound is effective because the cells with amplified Galanin, GALR2 and/or GALR3 are being eliminated or suppressed.

In one aspect of the inventions there are provided methods for diagnosing a cancer in a mammal, comprising: measuring the level of GALR2-GALR3 heterocomplex in a biological subject from a region of the mammal that is suspected to be precancerous or cancerous, thereby generating data for a test level; and comparing the test level to data for a control level, wherein an elevated test level of the biological subject relative to the control level indicates the presence of a cancer in the mammal.

In another aspect of the invention is there are provided methods for monitoring the efficacy of a therapeutic treatment regimen in a patient, comprising measuring the levels of GALR2-GALR3 heterocomplex in a first sample of cancer cells obtained from a patient; administering the treatment regimen to the patient; measuring the levels of GALR2-GALR3 heterocomplex in a second sample of cancer cells from the patient at a time following administration of the treatment regimen; and comparing the levels of GALR2-GALR3 heterocomplex in the first and the second samples, wherein data showing a decrease in the levels in the second sample relative to the first sample indicates that the treatment regimen is effective in the patient.

Still in another aspect of the invention there are provided methods for inhibiting cancer growth in a mammalian tissue, comprising contacting the tissue with an inhibitor of GALR2-GALR3 heterocomplex, wherein the inhibitor is an antibody that binds to GALR2-GALR3 heterocomplex or an antagonist to GALR2-GALR3 heterocomplex, such as a small molecule.

Yet in another aspect of the invention there are provided methods of making a pharmaceutical composition comprising: a) identifying a compound which is a modulator of GALR2-GALR3 heterocomplex; b) synthesizing the compound; and c) optionally mixing the compound with suitable additives.

Still in a further aspect of the invention there is provided a pharmaceutical composition comprising GALR2-GALR3 heterocomplex or a fragment thereof, wherein the fragment has functional characteristics of GALR2-GALR3 heterocomplex.

Still another aspect of the invention is to provide a pharmaceutical composition prepared by the methods described herein, wherein the composition comprises an antibody that blocks the oncogenic function or anti-apoptotic activity of Galanin, GALR2, GALR3, or GALR2-GALR3 heterocomplex.

Another aspect of the invention is to provide a pharmaceutical composition prepared by the methods described herein, wherein the composition comprises an antibody that binds to a cell overexpressing Galanin, GALR2, or GALR3 protein, thereby resulting in death of the cell.

Yet another aspect of the invention is to provide a pharmaceutical composition obtainable by the methods described herein, wherein the composition comprises a Galanin, GALR2, or GALR3-derived polypeptide or a fragment or a mutant thereof, wherein the polypeptide has inhibitory activity that blocks the oncogenic function or anti-apoptotic activity of Galanin, GALR2, GALR3, or GALR2-GALR3 heterocomplex.

In still a further aspect, the invention provide methods for inducing an immune response in a mammal comprising contacting the mammal with Galanin, GALR2, or GALR3 polypeptide or polynucleotide, or a fragment thereof, wherein the immune response produces antibodies and/or T cell immune response to protect the mammal from cancers, including ovarian cancer.

In another aspect, the invention provides isolated Galanin, GALR2, and GALR3 gene amplicons for diagnosing cancer and/or monitoring the efficacy of a cancer therapy, which comprises, for example, obtaining a biological test sample from a region in the tissue that is suspected to be precancerous or cancerous; obtaining a biological control sample from a region in the tissue or other tissues in the mammal that is normal; and detecting in both the biological test sample and the biological control sample the level of respective gene amplicons, wherein a level of amplification higher in the biological subject than that in the biological control sample indicates a precancerous or cancer condition in the tissue. In an alternative aspect the biological control sample may be obtained from a different individual or be a normalized value based on baseline values found in a population, such as two copies of a gene per cell.

In another aspect, the invention provides isolated Galanin, GALR2, and GALR3 gene amplicons, wherein the amplicons comprise a completely or partially amplified product of the respective gene, including a polynucleotide having at least about 90% sequence identity to Galanin, GALR2, or GALR3 gene, for example, SEQ ID NO: 1, a polynucleotide encoding the polypeptide set forth in SEQ ID NO:2, or a polynucleotide that is overexpressed in tumor cells having at least about 90% sequence identity to the polynucleotide of SEQ ID NO:1 or the polynucleotide encoding the polypeptide set forth in SEQ ID NO:2.

In yet another aspect, the present invention provides methods for modulating Galanin, GALR2, GALR3, or GALR2-GALR3 heterocomplex activities by contacting a biological subject from a region that is suspected to be precancerous or cancerous with a modulator of the Galanin, GALR2, GALR3, or GALR2-GALR3 heterocomplex protein, wherein the modulator is, for example, a small molecule.

In still another aspect, the present invention provides methods for modulating Galanin, GALR2, GALR3, or GALR2-GALR3 heterocomplex activities by contacting a biological subject from a region that is suspected to be precancerous or cancerous with a modulator of the Galanin, GALR2, GALR3, or GALR2-GALR3 heterocomplex protein, wherein said modulator partially or completely inhibits transcription of Galanin, GALR2, or GALR3 gene.

Another aspect of the invention is to provide methods of making pharmaceutical compositions comprising: identifying a compound which is an inhibitor of Galanin, GALR2, GALR3, or GALR2-GALR3 heterocomplex activity, including the oncogenic function or anti-apoptotic activity of Galanin, GALR2, GALR3, or GALR2-GALR3 heterocomplex; synthesizing the compound; and optionally mixing the compound with suitable additives.

Still another aspect of the invention is to provide pharmaceutical compositions obtainable by the methods described herein, wherein the composition comprises an antibody that blocks the oncogenic function or anti-apoptotic activity of Galanin, GALR2, GALR3, or GALR2-GALR3 heterocomplex.

Another aspect of the invention is to provide a pharmaceutical composition obtainable by the methods described herein, wherein the composition comprises an antibody that binds to a cell over-expressing Galanin, GALR2, GALR3, or GALR2-GALR3 heterocomplex protein, thereby resulting in death of the cell.

Yet another aspect of the invention is to provide pharmaceutical compositions obtainable by the methods described herein, wherein the composition comprises a Galanin-, GALR2-, GALR3-, or GALR2-GALR3 heterocomplex-derived polypeptide or a fragment or a mutant thereof, wherein the polypeptide has inhibitory activity that blocks the oncogenic function or anti-apoptotic activity of Galanin, GALR2, GALR3, or GALR2-GALR3 heterocomplex.

In still a further aspect, the invention provide methods for inducing an immune response in a mammal comprising contacting the mammal with Galanin, GALR2, or GALR3, polypeptide or polynucleotide, or a fragment thereof, wherein the immune response produces antibodies and/or T cell immune response to protect the mammal from cancers, including breast cancer, colon cancer, prostate cancer, and/or ovarian cancer.

Another aspect of the invention is to provide methods of administering siRNA to a patient in need thereof, wherein the siRNA molecule is delivered in the form of a naked oligonucleotide, sense molecule, antisense molecule, a decoy DNA molecule, or a vector, wherein the siRNA interacts with Galanin, GALR2, or GALR3 gene or their transcripts, wherein the vector is a plasmid, cosmid, bacteriophage, or a virus, wherein the virus is for example, a retrovirus, an adenovirus, or other suitable viral vector.

Yet in another aspect, the invention provide methods of administering decoy molecule to a patient in need thereof, wherein the molecule is delivered in the form of a naked oligonucleotide, sense molecule, antisense molecule, a decoy DNA molecule, or a vector, wherein the molecule interacts with Galanin, GALR2, or GALR3 gene, wherein the vector is a plasmid, cosmid, bacteriophage, or a virus, wherein the virus is for example, a retrovirus, an adenovirus, or other suitable viral vector.

In still a further aspect of the invention, Galanin, GALR2 and GALR3 decoys, antisense, triple helix forming molecules, and ribozymes can be administered concurrently or consecutively in any proportion; only two of the above can be administered concurrently or consecutively in any proportion; or they can be administered singly (that is, decoys, triple helix forming molecules, antisense or ribozymes targeting only one of Galanin, GALR2 or GALR3). Additionally, decoys, triple helix forming molecules, antisense and ribozymes having different sequences but directed against a given target (that is, Galanin, GALR2 or GALR3) can be administered concurrently or consecutively in any proportion, including equimolar proportions. Thus, as is apparent to the skilled person in view of the teachings herein, one could chose to administer 2 different Galanin decoys, triple helix forming molecules, antisense and/or ribozymes, 1 GALR2 decoy, triple helix forming molecule, antisense, ribozymes and/or 3 different GALR3 decoys, triple helix forming molecules, antisense and/or ribozymes in any proportion, including equimolar proportions, for example. Of course, other permutations and proportions can be employed by the person skilled in the art.

Still in another aspect, the invention provide methods of administering Galanin-siRNA and/or GALR2-siRNA and/or GALR3-siRNA to a patient in need thereof, wherein one or more of the above siRNA molecules are delivered in the form of a naked oligonucleotide, sense molecule, antisense molecule or a vector, wherein the siRNA(s) interact(s) with Galanin, GALR2, GALR3 or GALR2-GALR3 heterocomplex activity, wherein the vector is a plasmid, cosmid, bacteriophage or a virus, wherein the virus is for example, a retrovirus, an adenovirus, or other suitable viral vector. In other words, Galanin, GALR2 and GALR3 siRNAs can be administered concurrently or consecutively in any proportion; only two of the above can be administered concurrently or consecutively in any proportion; or they can be administered singly (that is, siRNAs targeting only one of Galanin, GALR2 or GALR3). Additionally, siRNAs having different sequences but directed against a given target (that is, Galanin, GALR2 or GALR3) can be administered concurrently or consecutively in any proportion, including equimolar proportions. Thus, as is apparent to the skilled person in view of the teachings herein, one could chose to administer 2 different Galanin siRNAs, 1 GALR2 siRNA and 3 different GALR3 siRNAs in any proportion, including equimolar proportions, for example. Of course, other permutations and proportions can be employed by the person skilled in the art. Additionally, siRNAs can be employed together with one or more of decoys, triple helix forming molecules, antisense, and ribozymes.

In another aspect, the present invention provide methods of blocking in vivo expression of a gene by administering a vector containing Galanin siRNA and/or GALR2 siRNA and/or GALR3 siRNA, wherein the siRNA interacts with Galanin and/or GALR2 and/or GALR3 activity, wherein the siRNA cause post-transcriptional silencing of Galanin and/or GALR2 and/or GALR3 gene in a mammalian cell, for example, a human cell.

Yet, in another aspect, the present invention provide methods of the treating cells ex vivo by administering a vector as described herein, wherein the vector is a plasmid, cosmid, bacteriophage, or a virus, such as a retrovirus or an adenovirus.

In its in vivo or ex vivo therapeutic applications, it is appropriate to administer siRNA/shRNA/decoy molecule using a viral or retroviral vector which enters the cell by transfection or infection. In particular, as a therapeutic product according to the invention, a vector can be a defective viral vector, such as an adenovirus, or a defective retroviral vector, such as a murine retrovirus.

Another aspect of the invention provide methods of screening a test molecule for Galanin, GALR2, GALR3, or GALR2-GALR3 heterocomplex antagonist activity comprising the steps of: contacting the molecule with a cancer cell; determining the level of Galanin, GALR2, GALR3, or GALR2-GALR3 heterocomplex in the cell, thereby generating data for a test level; and comparing the test level to a control level, wherein a decrease in Galanin, GALR2, GALR3, and/or GALR2-GALR3 heterocomplex level in the cell relative to the control indicates Galanin, GALR2, GALR3, and/or GALR2-GALR3 heterocomplex antagonist activity of the test molecule, wherein the level of Galanin, GALR2, GALR3, or GALR2-GALR3 heterocomplex is determined by reverse transcription and polymerase chain reaction (RT-PCR), Northern hybridization, or microarray analysis.

In another aspect, the invention provide methods of screening a test molecule for Galanin, GALR2, GALR3, or GALR2-GALR3 heterocomplex antagonist activity comprising the steps of: contacting the molecule with Galanin, GALR2, GALR3, or GALR2-GALR3 beterocomplex; and determining the effect of the test molecule on Galanin, GALR2, GALR3, or GALR2-GALR3 heterocomplex, wherein the level of Galanin, GALR2, GALR3, or GALR2-GALR3 heterocomplex, wherein the effect is determined via a binding assay.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 depicts the epicenter mapping of human chromosome region 22q13 amplicon, which includes GALR3 locus. The number of DNA copies for each sample is plotted on the Y-axis, and the X-axis corresponds to nucleotide position based on Human Genome Project working draft sequence (http://genome.ucsc.edu/goldenPath/aug2001Tracks.html).

FIG. 2 depicts the epicenter mapping of human chromosome region 17q25 amplicon, which includes GALR2 locus. The number of DNA copies for each sample is plotted on the Y-axis, and the X-axis corresponds to nucleotide position based on Human Genome Project working draft sequence (http://genome.ucsc.edu/goldenPath/aug2001Tracks.html).

FIG. 3 depicts the epicenter mapping of human chromosome region 11q13 amplicon, which includes Galanin locus. The number of DNA copies for each sample is plotted on the Y-axis, and the X-axis corresponds to nucleotide position based on Human Genome Project working draft sequence (http://genome.ucsc.edu/goldenPath/aug2001Tracks.html).

FIG. 4 shows animal tumorigenicity assay for the galanin gene. FIG. 4A is a control experiment using the vector-C8 cells and FIG. 4B shows the results of mice carrying galanin-C8 cells. All 5 mice carrying galanin-C8 cells developed tumors (FIG. 4B).

### DETAILED DESCRIPTION OF THE INVENTION

The present invention provides methods and compositions for the diagnosis, prevention, and treatment of tumors and cancers, for example, a lung cancer, a brain cancer, a breast cancer, a colon cancer, a prostate cancer, or an ovarian cancer, in mammals, for example, humans. The invention is based on the findings of novel traits of the Galanin, Galanin Receptor 2 (GALR2) and Galanin Receptor 3 (GALR3) genes. The Galanin, GALR3 and/or GALR2 genes and their expressed protein products can thus be used diagnostically or as targets for therapy; and, they can also be used to identify compounds useful in the diagnosis, prevention, and therapy of tumors and cancers (for example, a lung cancer, a brain cancer, a breast cancer, a colon cancer, a prostate cancer, or an ovarian cancer).

This invention provides that Galanin, and GALR3 are frequently amplified and overexpressed in human lung primary tumors. The present invention also discloses that a second galanin receptor (GALR2), is coamplified with GALR3 frequently in the same human lung, brain, breast, colon, prostate, and ovarian tumors, implying that both GALR2 and GALR3 are selected for during tumorigenesis and that a functionally important heterocomplex of GALR2-GALR3 exists.

This invention also provides that the ligand to the galanin receptors, Galanin, is frequently amplified (over 70%, 54/77 samples tested) and overexpressed (78%, 39/50 samples tested) in the lung tumors containing amplified and overexpressed galanin receptors. These findings indicate that the galanin autocrine loop is under genetic pressure in lung cancers to be over-represented, implicating an important role for Galanin and its receptors in lung cancers.

Definitions:

A “cancer” in an animal refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

The phrase “detecting a cancer” or “diagnosing a cancer” refers to determining the presence or absence of cancer or a precancerous condition in an animal. “Detecting a cancer” also can refer to obtaining indirect evidence regarding the likelihood of the presence of precancerous or cancerous cells in the animal or assessing the predisposition of a patient to the development of a cancer. Detecting a cancer can be accomplished using the methods of this invention alone, in combination with other methods, or in light of other information regarding the state of health of the animal.

A “tumor,” as used herein, refers to all neoplastic cell growth and proliferation, whether malignant or benign, and all precancerous and cancerous cells and tissues.

The term “precancerous” refers to cells or tissues having characteristics relating to changes that may lead to malignancy or cancer. Examples include adenomatous growths in breast tissue, or conditions, for example, dysplastic nevus syndrome, a precursor to malignant melanoma of the skin. Examples also include, abnormal neoplastic, in addition to dysplastic nevus syndromes, polyposis syndromes, prostatic dysplasia, and other such neoplasms, whether the precancerous lesions are clinically identifiable or not.

A “differentially expressed gene transcript”, as used herein, refers to a gene, including an oncogene, transcript that is found in different numbers of copies in different cell or tissue types of an organism having a tumor or cancer, for example, breast cancer, colon cancer, lung cancer, brain cancer, prostate cancer, or ovarian cancer, compared to the numbers of copies or state of the gene transcript found in the cells of the same tissue in a healthy organism, or in the cells of the same tissue in the same organism. Multiple copies of gene transcripts may be found in an organism having the tumor or cancer, while fewer copies of the same gene transcript are found in a healthy organism or healthy cells of the same tissue in the same organism, or vice-versa.

A “differentially expressed gene,” can be a target, fingerprint, or pathway gene. For example, a “fingerprint gene”, as used herein, refers to a differentially expressed gene whose expression pattern can be used as a prognostic or diagnostic marker for the evaluation of tumors and cancers, or which can be used to identify compounds useful for the treatment of tumors and cancers, for example, breast cancer, colon cancer, lung cancer, brain cancer, prostate cancer, or ovarian cancer. For example, the effect of a compound on the fingerprint gene expression pattern normally displayed in connection with tumors and cancers can be used to evaluate the efficacy of the compound as a tumor and cancer treatment, or can be used to monitor patients undergoing clinical evaluation for the treatment of tumors and cancer.

A “fingerprint pattern”, as used herein, refers to a pattern generated when the expression pattern of a series (which can range from two up to all the fingerprint genes that exist for a given state) of fingerprint genes is determined. A fingerprint pattern may also be referred to as an “expression profile”. A fingerprint pattern or expression profile can be used in the same diagnostic, prognostic, and compound identification methods as the expression of a single fingerprint gene.

A “target gene”, as used herein, refers to a differentially expressed gene in which modulation of the level of gene expression or of gene product activity prevents and/or ameliorates tumor and cancer, for example, breast cancer, colon cancer, lung cancer, brain cancer, prostate cancer, or ovarian cancer, symptoms. Thus, compounds that modulate the expression of a target gene, the target genes, or the activity of a target gene product can be used in the diagnosis, treatment or prevention of tumors and cancers. A particular target gene of the present invention is the Galanin, GALR2, or GALR3 gene.

In general, a “gene” is a region on the genome that is capable of being transcribed to an RNA that either has a regulatory function, a catalytic function, and/or encodes a protein. An eukaryotic gene typically has introns and exons, which may organize to produce different RNA splice variants that encode alternative versions of a mature protein. The skilled artisan will appreciate that the present invention encompasses all Galanin-, GALR2-, and GALR3-encoding transcripts that may be found, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. A “fragment” of a gene, including an oncogene, can be any portion from the gene, which may or may not represent a functional domain, for example, a catalytic domain, a DNA binding domain, etc. A fragment may preferably include nucleotide sequences that encode for at least 25 contiguous amino acids, and preferably at least about 30, 40, 50, 60, 65, 70, 75 or more contiguous amino acids or any integer thereabout or therebetween.

“Pathway genes”, as used herein, are genes that encode proteins or polypeptides that interact with other gene products involved in tumors and cancers. Pathway genes also can exhibit target gene and/or fingerprint gene characteristics.

A “detectable” RNA expression level, as used herein, means a level that is detectable by standard techniques currently known in the art or those that become standard at some future time, and include for example, differential display, RT (reverse transcriptase)-coupled polymerase chain reaction (PCR), Northern Blot, and/or RNase protection analyses. The degree of differences in expression levels need only be large enough to be visualized or measured via standard characterization techniques.

As used herein, the term “transformed cell” means a cell into which (or into an ancestor of which) a nucleic acid molecule encoding a polypeptide of the invention has been introduced, by means of, for example, recombinant DNA techniques or viruses.

The nucleic acid molecules of the invention, for example, the Galanin gene or its subsequences, can be inserted into a vector, as described below, which will facilitate expression of the insert. The nucleic acid molecules and the polypeptides they encode can be used directly as diagnostic or therapeutic agents, or can be used (directly in the case of the polypeptide or indirectly in the case of a nucleic acid molecule) to generate antibodies that, in turn, are clinically useful as a therapeutic or diagnostic agent. Accordingly, vectors containing the nucleic acids of the invention, cells transfected with these vectors, the polypeptides expressed, and antibodies generated against either the entire polypeptide or an antigenic fragment thereof, are among the aspects of the invention.

A “structural gene” is a DNA sequence that is transcribed into messenger RNA (mRNA) which is then translated into a sequence of amino acids characteristic of a specific polypeptide.

An “isolated DNA molecule” is a fragment of DNA that has been separated from the chromosomal or genomic DNA of an organism. Isolation also is defined to connote a degree of separation from original source or surroundings. For example, a cloned DNA molecule encoding an avidin gene is an isolated DNA molecule. Another example of an isolated DNA molecule is a chemically-synthesized DNA molecule, or enzymatically-produced cDNA, that is not integrated in the genomic DNA of an organism. Isolated DNA molecules can be subjected to procedures known in the art to remove contaminants such that the DNA molecule is considered purified, that is towards a more homogeneous state.

“Complementary DNA” (cDNA), often referred to as “copy DNA”, is a single-stranded DNA molecule that is formed from an mRNA template by the enzyme reverse transcriptase. Typically, a primer complementary to portions of the mRNA is employed for the initiation of reverse transcription. Those skilled in the art also use the term “cDNA” to refer to a double-stranded DNA molecule that comprises such a single-stranded DNA molecule and its complement DNA strand.

The term “expression” refers to the biosynthesis of a gene product. For example, in the case of a structural gene, expression involves transcription of the structural gene into mRNA and the translation of mRNA into one or more polypeptides.

The term “amplification” refers to amplification, duplication, multiplication, or multiple expression of nucleic acids or a gene, in vivo or in vitro, yielding about 2.5 fold or more copies. For example, amplification of the Galanin, GALR2, or GALR3 gene resulting in a copy number greater than or equal to 2.5 is deemed to have been amplified. However, an increase in Galanin, GALR2, or GALR3 gene copy number less than 2.5 fold can still be considered as an amplification of the gene. The 2.5 fold figure is due to current detection limit, rather than a biological state.

The term “amplicon” refers to an amplification product containing one or more genes, which can be isolated from a precancerous or a cancerous cell or a tissue. Galanin, GALR2, or GALR3 amplicon is a result of amplification, duplication, multiplication, or multiple expression of nucleic acids or a gene, in vivo or in vitro. “Amplicon”, as defined herein, also includes a completely or partially amplified Galanin and/or GALR2 and/or GALR3 genes. For example, an amplicon comprising a polynucleotide having at least about 90% sequence identity to SEQ ID NO:1 or a discernible fragment thereof.

A “cloning vector” is a nucleic acid molecule, for example, a plasmid, cosmid, or bacteriophage that has the capability of replicating autonomously in a host cell. Cloning vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed with the cloning vector. Marker genes include genes that provide tetracycline resistance or ampicillin resistance, for example.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

“Antisense RNA”: In eukaryotes, RNA polymerase catalyzes the transcription of a structural gene to produce mRNA. A DNA molecule can be designed to contain an RNA polymerase template in which the RNA transcript has a sequence that is complementary to that of a preferred mRNA. The RNA transcript is termed an “antisense RNA”. Antisense RNA molecules can inhibit mRNA expression (for example, Rylova et al., Cancer Res, 62(3):801-8, 2002; Shim et al., Int. J. Cancer, 94(1):6-15, 2001).

“Antisense DNA or DNA decoy or decoy molecule”: With respect to a first nucleic acid molecule, a second DNA molecule or a second chimeric nucleic acid molecule that is created with a sequence, which is a complementary sequence or homologous to the complementary sequence of the first molecule or portions thereof, is referred to as the “antisense DNA or DNA decoy or decoy molecule” of the first molecule. The term “decoy molecule” also includes a nucleic molecule, which may be single or double stranded, that comprises DNA or PNA (peptide nucleic acid) (Mischiati et al., Int. J. Mol. Med., 9(6):633-9, 2002), and that contains a sequence of a protein binding site, preferably a binding site for a regulatory protein and more preferably a binding site for a transcription factor. Applications of antisense nucleic acid molecules, including antisense DNA and decoy DNA molecules are known in the art, for example, Morishita et al., Ann. N Y Acad. Sci., 947:294-301, 2001; Andratschke et al., Anticancer Res, 21:(5)3541-3550, 2001. Antisense DNA or PNA molecules can inhibit, block, or regulate function and/or expression of a Galanin, a GALR2, or a GALR3 gene.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene or coding region is said to be “operably linked to” or “operatively linked to” the regulatory elements, meaning that the gene or coding region is controlled or influenced by the regulatory element.

“Sequence homology” is used to describe the sequence relationships between two or more nucleic acids, polynucleotides, proteins, or polypeptides, and is understood in the context of and in conjunction with the terms including: (a) reference sequence, (b) comparison window, (c) sequence identity, (d) percentage of sequence identity, and (e) substantial identity or “homologous.”

(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and most preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and most preferably about 100 nucleotides or about 300 nucleotides or any integer thereabout or therebetween.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Generally, the comparison window is at least 20 contiguous nucleotides in length, and optionally can be 30, 40, 50, 100, or longer. Those of skill in the art understand that to avoid a misleadingly high similarity to a reference sequence due to inclusion of gaps in the polynucleotide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482 (1981); by the homology alignment algorithm of Needleman and Wunsch, J. Mol. Biol., 48: 443 (1970); by the search for similarity method of Pearson and Lipman, Proc. Natl. Acad. Sci. USA, 8: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters. Altschul et al., Nucleic Acids Res, 2:3389-3402, 1997. It is to be understood that default settings of these parameters can be readily changed as needed in the future.

As those ordinary skilled in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163, 1993) and XNU (Clayerie and States, Comput. Chem., 17:191-1, 1993) low-complexity filters can be employed alone or in combination.

(c) “Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The term “substantial identity” or “homologous” in their various grammatical forms means that a polynucleotide comprises a sequence that has a desired identity, for example, at least 60% identity, preferably at least 70% sequence identity, more preferably at least 80%, still more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

(e) (ii) The terms “substantial identity” or “homologous” in their various grammatical forms in the context of a peptide indicates that a peptide comprises a sequence that has a desired identity, for example, at least 60% identity, preferably at least 70% sequence identity to a reference sequence, more preferably 80%, still more preferably 85%, most preferably at least 90% or 95% sequence identity to the reference sequence over a specified comparison window. Preferably, optimal alignment is conducted using the homology alignment algorithm of Needleman and Wunsch, J. Mol. Biol., 48:443, 1970. An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine, and others as known to the skilled person.

The term “Galanin” refers to Galanin nucleic acid (DNA and RNA), protein (or polypeptide), their polymorphic variants, alleles, mutants, and interspecies homologs that have (i) substantial nucleotide sequence homology with the nucleotide sequence of the GenBank Accession Number A28025, human preprogalanin cDNA sequence, SEQ ID NO: 1, [Unigene clusters ID (http://www.ncbi.nlm.nih.gov/UniGene/): Hs. 1907, Human Galanin.]; or (ii) substantial sequence homology with the encoding amino acid sequence GenBank Accession Number CAA01907, amino acid sequence of human preprogalanin, SEQ ID NO: 2; or (iii) substantial sequence homology with the encoding amino acid sequence GenBank Accession Number AAB20740, human galanin peptide sequence, SEQ ID NO: 3.

Galanin polynucleotide or polypeptide sequence is typically from a mammal including, but not limited to, human, rat, mouse, hamster, cow, pig, horse, sheep, or any mammal. A “Galanin polynucleotide” and a “Galanin polypeptide,” may be either naturally occurring, recombinant, or synthetic (for example via chemical synthesis).

The term “GALR2” refers to GALR2 nucleic acid (DNA and RNA), protein (or polypeptide), their polymorphic variants, alleles, mutants, and interspecies homologs that have (i) substantial nucleotide sequence homology with the nucleotide sequence of the GenBank Accession Number NM_{—}003857, human galanin receptor 2 (GALR2); or (ii) substantial sequence homology with the encoding amino acid sequence [SWISS-PROT Accession Number 043603. Unigene clusters GALR2 is Hs. 158351].

GALR2 polynucleotide or polypeptide sequence is typically from a mammal including, but not limited to, human, rat, mouse, hamster, cow, pig, horse, sheep, or any mammal. A “GALR2 polynucleotide” and a “GALR2 polypeptide,” may be either naturally occurring, recombinant, or synthetic (for example via chemical synthesis).

The term “GALR3” refers to GALR3 nucleic acid (DNA and RNA), protein (or polypeptide), their polymorphic variants, alleles, mutants, and interspecies homologs that have (i) substantial nucleotide sequence homology with the nucleotide sequence of the GenBank Accession NM_{—}003614, human galanin receptor 3 (GALR3); or (ii) substantial sequence homology with the encoding amino acid sequence [SWISS-PROT Accession Number 060755. Unigene clusters GALR3 is Hs. 158353 contains 2 ESTs with Accession Number BF 116239 and BF512731].

GALR3 polynucleotide or polypeptide sequence is typically from a mammal including, but not limited to, human, rat, mouse, hamster, cow, pig, horse, sheep, or any mammal. A “GALR3 polynucleotide” and a “GALR3 polypeptide,” may be either naturally occurring, recombinant, or synthetic (for example via chemical synthesis).

The term “GALR2-GALR3 heterocomplex” refers to a complex, mixture, or combination of GALR2 and GALR3 proteins or polypeptides, their polymorphic variants, alleles, mutants, and interspecies homologs that have (i) substantial nucleotide sequence homology with the GALR3 nucleotide sequence of the GenBank Accession Number NM_{—}003614, human galanin receptor 3 (GALR3) or GALR2 nucleotide sequence of GenBank Accession Number NM_{—}003857, human galanin receptor 2 (GALR2); or (ii) substantial sequence homology with the encoding GALR3 amino acid sequence [SWISS-PROT Accession Number 060755. Unigene clusters GALR3 is Hs. 158353 contains 2 ESTs with Accession Number BF 116239 and BF512731], or GALR2 amino acid sequence [SWISS-PROT Accession Number 043603. Unigene clusters GALR2 is Hs. 158351]. “GALR2-GALR3 heterocomplex” also can be obtained from a mixture or combination of GALR2 and GALR3 nucleic acids (DNA or RNA, or fragments thereof) in a construct or an expression vector that expresses proteins or polypeptides individually or as transcription or translational fusions, their polymorphic variants, alleles, mutants, and interspecies homologs that have (i) substantial nucleotide sequence homology with the GALR3 nucleotide sequence of the GenBank Accession Number NM 003614, human galanin receptor 3 (GALR3) or GALR2 nucleotide sequence of GenBank Accession Number NM_{—}003857, human galanin receptor 2 (GALR2); or (ii) substantial sequence homology with the encoding GALR3 amino acid sequence [SWISS-PROT Accession Number 060755. Unigene clusters GALR3 is Hs. 158353 contains 2 ESTs with Accession Number BF116239 and BF512731], or GALR2 amino acid sequence [SWISS-PROT Accession Number 043603. Unigene clusters GALR2 isHs. 158351].

GALR2-GALR3 heterocomplex is typically from a mammal including, but not limited to, human, rat, mouse, hamster, cow, pig, horse, sheep, or any mammal.

“Biological subject” as used herein refers to a target biological object obtained, reached, or collected in vivo or in situ, that contains or is suspected of containing nucleic acids or polypeptides of Galanin, GALR2, or GALR3, or GALR2-GALR3 protein heterocomplex. A biological subject is typically of eukaryotic nature, for example, insects, protozoa, birds, fish, reptiles, and preferably a mammal, for example, rat, mouse, cow, dog, guinea pig, or rabbit, and most preferably a primate, for example, chimpanzees, or humans such as a patient in need of diagnostic review, treatment and/or monitoring of therapy.

“Biological sample” as used herein refers to a sample obtained from a biological subject, including sample of biological tissue or fluid origin, obtained, reached, or collected in vivo or in situ, that contains or is suspected of containing nucleic acids or polypeptides of Galanin, GALR2, or GALR3, or GALR2-GALR3 protein heterocomplex. Such samples include, but are not limited to, organs, tissues, fractions and cells isolated from mammals including, humans such as a patient, mice, and rats. Biological samples may also include sections of the biological sample including tissues, for example, frozen sections taken for histologic purposes. A biological sample is typically of an eukaryotic origin, for example, insects, protozoa, birds, fish, reptiles, and preferably a mammal, for example, rat, mouse, cow, dog, guinea pig, or rabbit, and most preferably a primate, for example, chimpanzees or humans.

“Providing a biological subject or sample” means to obtain a biological subject in vivo or in situ, including tissue or cell sample for use in the methods described in the present invention. Most often, this will be done by removing a sample of cells from an animal, but can also be accomplished in vivo or in situ or by using previously isolated cells (for example, isolated from another person, at another time, and/or for another purpose).

A “control sample” refers to a sample of biological material representative of healthy, cancer-free animals. The level of Galanin, GALR2, GALR3, or GALR2-GALR3 heterocomplex in a control sample, or the encoding corresponding gene copy number, is desirably typical of the general population of normal, cancer-free animals of the same species. This sample either can be collected from an animal for the purpose of being used in the methods described in the present invention or it can be any biological material representative of normal, cancer-free animals obtained for other reasons but nonetheless suitable for use in the methods of this invention. A control sample can also be obtained from normal tissue from the animal that has cancer or is suspected of having cancer. A control sample also can refer to a given level of Galanin, GALR2, GALR3, or GALR2-GALR3 heterocomplex, representative of the cancer-free population, that has been previously established based on measurements from normal, cancer-free animals. Alternatively, a biological control sample can refer to a sample that is obtained from a different individual or be a normalized value based on baseline data obtained from a population. Further, a control sample can be defined by a specific age, sex, ethnicity or other demographic parameters. In some situations, the control is implicit in the particular measurement. An example of an implicit control is where a detection method can only detect Galanin, GALR2, GALR3, or GALR2-GALR3 heterocomplex, or the corresponding gene copy number, when a level higher than that typical of a normal, cancer-free animal is present. A typical control level for a gene is two copies per cell. Another example is in the context of an immunohistochemical assay where the control level for the assay is known. Other instances of such controls are within the knowledge of the skilled person.

“Data” includes, but is not limited to, information obtained that relates to “Biological Sample” or “Control Sample”, as described above, wherein the information is applied in generating a test level for diagnostics, prevention, monitoring or therapeutic use. The present invention relates to methods for comparing and compiling data wherein the data is stored in electronic or paper formats. Electronic format can be selected from the group consisting of electronic mail, disk, compact disk (CD), digital versatile disk (DVD), memory card, memory chip, ROM or RAM, magnetic optical disk, tape, video, video clip, microfilm, internet, shared network, shared server and the like; wherein data is displayed, transmitted or analyzed via electronic transmission, video display, telecommunication, or by using any of the above stored formats; wherein data is compared and compiled at the site of sampling specimens or at a location where the data is transported following a process as described above.

“Overexpression” of a Galanin, GALR2, or GALR3 genes or an “increased,” or “elevated,” level of a Galanin, GALR2, or GALR3 polynucleotide or protein or GALR2-GALR3 protein heterocomplex refers to a level of Galanin, GALR2, or GALR3 polynucleotide or polypeptide or GALR2-GALR3 protein heterocomplex that, in comparison with a control level of Galanin, GALR2, GALR3, or GALR2-GALR3 heterocomplex, respectively, is detectably higher. Comparison may be carried out by statistical analyses on numeric measurements of the expression; or, it may be done through visual examination of experimental results by qualified researchers.

A level of Galanin, GALR2, or GALR3 polypeptide or polynucleotide, or GALR2-GALR3 heterocomplex, that is “expected” in a control sample refers to a level that represents a typical, cancer-free sample, and from which an elevated, or diagnostic, presence of Galanin, GALR2, or GALR3 polypeptide or polynucleotide, or GALR2-GALR3 heterocomplex, can be distinguished. Preferably, an “expected” level will be controlled for such factors as the age, sex, medical history, etc. of the mammal, as well as for the particular biological subject being tested.

The phrase “functional effects” in the context of an assay or assays for testing compounds that modulate Galanin, GALR2, GALR3, or GALR2-GALR3 heterocomplex activity includes the determination of any parameter that is indirectly or directly under the influence of Galanin, GALR2, GALR3, or GALR2-GALR3 heterocomplex, for example, a functional, physical, or chemical effect, such as the protease activity, the ability to induce gene amplification or overexpression in cancer cells, and to aggravate cancer cell proliferation. “Functional effects” include in vitro, in vivo, and ex vivo activities.

“Determining the functional effect” refers to assaying for a compound that increases or decreases a parameter that is indirectly or directly under the influence of Galanin, GALR2, GALR3, or GALR2-GALR3 heterocomplex, for example, functional, physical, and chemical effects. Such functional effects can be measured by any means known to those skilled in the art, for example, changes in spectroscopic characteristics (for example, fluorescence, absorbance, refractive index), hydrodynamic (for example, shape), chromatographic, or solubility properties for the protein, measuring inducible markers or transcriptional activation of Galanin, GALR2, or GALR3; measuring binding activity or binding assays, for example, substrate binding, and measuring cellular proliferation; measuring signal transduction; measuring cellular transformation, etc.

“Inhibitors,” “activators,” “modulators,” and “regulators” refer to molecules that activate, inhibit, modulate, regulate and/or block an identified function. For example, referring to oncogenic function or anti-apoptotic activity of Galanin, GALR2, GALR3, or GALR2-GALR3 heterocomplex, such molecules may be identified using in vitro and in vivo assays of Galanin, GALR2, or GALR3, respectively. Inhibitors are compounds that partially or totally block Galanin, GALR2, GALR3, or GALR2-GALR3 heterocomplex, activity, decrease, prevent, or delay its activation, or desensitize its cellular response. This may be accomplished by binding to Galanin, GALR2, GALR3, or GALR2-GALR3 heterocomplex proteins directly or via other intermediate molecules, such as a receptor. An antagonist or an antibody that blocks Galanin, GALR2, GALR3, or GALR2-GALR3 heterocomplex activity, including inhibition of oncogenic function or anti-apoptotic activity of Galanin, GALR2, GALR3, or GALR2-GALR3 heterocomplex, is considered to be such an inhibitor. Activators are compounds that bind to Galanin, GALR2, GALR3, or GALR2-GALR3 heterocomplex protein directly or via other intermediate molecules, such as their receptor, thereby increasing or enhancing its activity, stimulating or accelerating its activation, or sensitizing its cellular response. An agonist of Galanin, GALR2, GALR3, or GALR2-GALR3 heterocomplex is considered to be such an activator. A modulator can be an inhibitor or activator. A modulator may or may not bind Galanin, GALR2, GALR3, or GALR2-GALR3 heterocomplex or their receptor directly; it affects or changes the activity or activation of Galanin, GALR2, GALR3, or GALR2-GALR3 heterocomplex, or the cellular sensitivity to Galanin, GALR2, GALR3, or GALR2-GALR3 heterocomplex. A modulator also may be a compound, such as a small molecule, that inhibits transcription of Galanin, GALR2, or GALR3 mRNA. A regulator of Galanin, GALR2, or GALR3 gene includes any element, for example, nucleic acid, peptide, polypeptide, protein, peptide nucleic acid or the like, that influence and/or control the transcription/expression of Galanin, GALR2, or GALR3 gene or its coding region.

The group of inhibitors, activators, modulators and regulators of this invention also includes genetically modified versions of Galanin, GALR2, GALR3, or GALR2-GALR3 heterocomplex, for example, versions with altered activity. The group thus is inclusive of the naturally occurring protein as well as synthetic ligands, antagonists, agonists, antibodies, small chemical molecules and the like.

“Assays for inhibitors, activators, or modulators” refer to experimental procedures including, for example, expressing Galanin, GALR2, GALR3, or GALR2-GALR3 heterocomplex in vitro, in cells, applying putative inhibitor, activator, modulator, or regulator compounds, and then determining the functional effects on Galanin, GALR2, GALR3, or GALR2-GALR3 heterocomplex activity or transcription, as described above. Samples that contain or are suspected of containing Galanin, GALR2, GALR3, or GALR2-GALR3 heterocomplex are treated with a potential activator, inhibitor, or modulator. The extent of activation, inhibition, or change is examined by comparing the activity measurement from the samples of interest to control samples. A threshold level is established to assess activation or inhibition. For example, inhibition of a Galanin, GALR2, GALR3, or GALR2-GALR3 heterocomplex polypeptide is considered achieved when the Galanin, GALR2, GALR3, or GALR2-GALR3 heterocomplex activity value relative to the control is 80% or lower. Similarly, activation of a Galanin, GALR2, GALR3, or GALR2-GALR3 heterocomplex polypeptide is considered achieved when the Galanin, GALR2, GALR3, or GALR2-GALR3 heterocomplex activity value relative to the control is two or more fold higher.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. Various levels of purity may be applied as needed according to this invention in the different methodologies set forth herein; the customary purity standards known in the art may be used if no standard is otherwise specified.

An “isolated nucleic acid molecule” can refer to a nucleic acid molecule, depending upon the circumstance, that is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res, 19:081, 1991; Ohtsuka et al., J. Biol. Chem., 260:2600-2608, 1985; Rossolini et al., Mol. Cell Probes, 8:91-98, 1994). The term nucleic acid can be used interchangeably with gene, cDNA, mRNA, oligonucleotide, and polynucleotide.

A “host cell” is a naturally occurring cell or a transformed cell that contains an expression vector and supports the replication or expression of the expression vector. Host cells may be cultured cells, explants, cells in vivo, and the like. Host cells may be prokaryotic cells, for example, E. coli, or eukaryotic cells, for example, yeast, insect, amphibian, or mammalian cells, for example, CHO, HeLa, and the like.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, γ-carboxyglutamate, and O-phosphoserine, phosphothreonine. “Amino acid analogs” refer to compounds that have the same basic chemical structure as a naturally occurring amino acid, i.e., a carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid. Amino acids and analogs are well known in the art.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” apply to both amino acid and nucleic acid sequences. With respect to particular nucleic acid sequences, conservatively modified variants refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and UGG (tryptophan), can be modified conservatively to yield a functionally-identical peptide or protein molecule.

As to amino acid sequences, one skilled in the art will recognize that substitutions, deletions, or additions to a polypeptide or protein sequence which alter, add or delete a single amino acid or a small number (typically less than about ten) of amino acids is a “conservatively modified variant” where the alteration results in the substitution of an amino acid with a chemically similar amino acid. Conservative substitutions are well known in the art and include, for example, the changes of: alanine to serine; arginine to lysine; asparigine to glutamine or histidine; aspartate to glutamate; cysteine to serine; glutamine to asparigine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine. Other conservative and semi-conservative substitutions are known in the art and can be employed in practice of the present invention.

The terms “protein”, “peptide” and “polypeptide” are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides of the invention can be chemically synthesized or synthesized by recombinant DNA methods; or, they can be purified from tissues in which they are naturally expressed, according to standard biochemical methods of purification.

Also included in the invention are “functional polypeptides,” which possess one or more of the biological functions or activities of a protein or polypeptide of the invention. These functions or activities include the ability to bind some or all of the proteins which normally bind to Galanin, GALR2, GALR3, or GALR2-GALR3 heterocomplex protein.

The functional polypeptides may contain a primary amino acid sequence that has been modified from that considered to be the standard sequence of Galanin, GALR2, or GALR3 described herein. Preferably these modifications are conservative amino acid substitutions, as described herein.

A “label” or a “detectable moiety” is a composition that when linked with the nucleic acid or protein molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. A “labeled nucleic acid or oligonucleotide probe” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

The phrase “stringent hybridization conditions” refers to conditions under which a probe will hybridize to its target complementary sequence, typically in a complex mixture of nucleic acids, but to no other sequences. Stringent conditions are sequence-dependent and circumstance-dependent; for example, longer sequences can hybridize with specificity at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

Exemplary stringent hybridization conditions can be as following, for example: 50% formamide, 5×SSC and 1% SDS, incubating at 42° C., or 5×SSC and 1% SDS, incubating at 65° C., with wash in 0.2×SSC and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C. to 95° C. for 30 sec. to 2 min., an annealing phase lasting 30 sec. to 2 min., and an extension phase of about 72° C. for 1 to 2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in lx SSC at 45° C. A positive hybridization is at least twice background. Those of ordinary skill will readily recognize that alternative hybridization and wash conditions can be utilized to provide conditions of similar stringency.

The terms “about” or “approximately” in the context of numerical values and ranges refers to values or ranges that approximate or are close to the recited values or ranges such that the invention can perform as intended, such as having a desired amount of nucleic acids or polypeptides in a reaction mixture, as is apparent to the skilled person from the teachings contained herein. This is due, at least in part, to the varying properties of nucleic acid compositions, age, race, gender, anatomical and physiological variations and the inexactitude of biological systems. Thus these terms encompass values beyond those resulting from systematic error.

“Antibody” refers to a polypeptide comprising a framework region encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 2 kDa) and one “heavy” chain (up to about 70 kDa). Antibodies exist, for example, as intact immunoglobulins or as a number of well-characterized fragments produced by digestion with various peptidases. While various antibody fragments are defined in terms of the digestion of an intact antibody, one of skill in the art will appreciate that such fragments may be synthesized de novo chemically or via recombinant DNA methodologies. Thus, the term antibody, as used herein, also includes antibody fragments produced by the modification of whole antibodies, those synthesized de novo using recombinant DNA methodologies (for example, single chain Fv), humanized antibodies, and those identified using phage display libraries (see, for example, Knappik et al., J. Mol. Biol., 296:57-86, 2000; McCafferty et al., Nature, 348:2-4, 1990), for example. For preparation of antibodies—recombinant, monoclonal, or polyclonal antibodies—any technique known in the art can be used with this invention (see, for example, Kohler & Milstein, Nature, 256(5517):495-497, 1975; Kozbor et al., Immunology Today, 4:72, 1983; Cole et al., pp. 77-96 in Monoclonal Antibodies and Cancer Therapy, Alan R. Liss, Inc., 1998).

Techniques for the production of single chain antibodies (See U.S. Pat. No. 4,946,778) can be adapted to produce antibodies to polypeptides of this invention. Transgenic mice, or other organisms, for example, other mammals, may be used to express humanized antibodies. Phage display technology can also be used to identify antibodies and heteromeric Fab fragments that specifically bind to selected antigens (see, for example, McCafferty et al., Nature, 348:2-4, 1990; Marks et al., Biotechnology, 10(7):779-783, 1992).

The term antibody is used in the broadest sense including agonist, antagonist, and blocking or neutralizing antibodies.

“Blocking antibody” is a type of antibody, as described above, that refers to a polypeptide comprising variable and framework regions encoded by an immunoglobulin gene or fragments, homologues, analogs or mimetics thereof that specifically binds and blocks biological activities of an antigen; for example, a blocking antibody to Galanin, GALR2, or GALR3 blocks the oncogenic function or anti-apoptotic activity of Galanin, GALR2, or GALR3 genes, respectively. A blocking antibody binds to critical regions of a polypeptide and thereby inhibits its function. Critical regions include protein-protein interaction sites, such as active sites, functional domains, ligand binding sites, and recognition sites. Blocking antibodies may be induced in mammals, for example in human, by repeated small injections of antigen, too small to produce strong hypersensitivity reactions. See Bellanti J A, Immunology, W B Saunders Co., p.131-368 (1971). Blocking antibodies play an important role in blocking the function of a marker protein and inhibiting tumorigenic growth. See, for example, Jopling et al., J. Biol. Chem., 277(9):6864-73 (2002); Drebin et al., Cell, 41(3):697-706 (1985); Drebin et al., Proc. Natl. Acad. Sci. USA, 83(23):9129-33 (1986).

The term “tumor-cell killing” by anti-Galanin, -GALR2, -GALR3 or -GALR2-GALR3 heterocomplex blocking antibodies herein is meant any inhibition of tumor cell proliferation by means of blocking a function or binding to block a pathway related to tumor-cell proliferation. For example, anti-epidermal growth factor receptor monoclonal antibodies inhibit A431 tumor cell proliferation by blocking an autocrine pathway. See Mendelsohn et al., Trans Assoc Am Physicians, 100: 173-8 (1987); Masui et al., Cancer Res, 44(3):1002-7 (1984).

The term “Galanin-, GALR2-, GALR3-, or GALR2-GALR3 heterocomplex-oncogenic function-blocking antibody” herein is meant an anti-human Galanin-, GALR2-, GALR3- or GALR2-GALR3 heterocomplex- antibody whose interaction with the Galanin, GALR2, or GALR3 protein, or GALR2-GALR3 heterocomplex inhibits the oncogenic function or anti-apoptotic activity of the protein, mediates tumor-cell killing mechanisms, or inhibits tumor-cell proliferation. In contrast to antibodies that merely bind to tumor cells expressing Galanin, GALR2, or GALR3, blocking antibodies against Galanin, GALR2, GALR3, or GALR2-GALR3 heterocomplex mediate tumor-cell killing by mechanisms related to the oncogenic function or anti-apoptotic activity of Galanin, GALR2, GALR3, or GALR2-GALR3 heterocomplex. See Drebin et al., Proc. Natl. Acad. Sci. USA, 83(23):9129-33 (1986) for inhibition of tumorigenic growth; and Mendelsohn et al., Trans Assoc Am Physicians, 100:173-8 (1987), for an example of antibody-mediated anti-proliferative activity.

An “anti-Galanin” antibody is an antibody or antibody fragment that specifically binds a polypeptide encoded by a Galanin gene, cDNA, or a subsequence thereof. Anti-Galanin antibody also includes a blocking antibody that inhibits oncogenic function or anti-apoptotic activity of Galanin or mediates anti-proliferative activity on tumor-cell growth.

An “anti-GALR2” antibody is an antibody or antibody fragment that specifically binds a polypeptide encoded by a GALR2 gene, cDNA, or a subsequence thereof. Anti-GALR2 antibody also includes a blocking antibody that inhibits oncogenic function or anti-apoptotic activity of GALR2 or mediates anti-proliferative activity on tumor-cell growth.

An “anti-GALR3” antibody is an antibody or antibody fragment that specifically binds a polypeptide encoded by a GALR3 gene, cDNA, or a subsequence thereof. Anti-GALR3 antibody also includes a blocking antibody that inhibits oncogenic function or anti-apoptotic activity of GALR3 or mediates anti-proliferative activity on tumor-cell growth.

An “anti-GALR2-GALR3 heterocomplex” antibody is an antibody or antibody fragment that specifically binds a GALR2-GALR3 heterocomplex or a fragment thereof. Anti-GALR2-GALR3 heterocomplex antibody also includes a blocking antibody that inhibits oncogenic function or anti-apoptotic activity of GALR2-GALR3 heterocomplex or mediates anti-proliferative activity on tumor-cell growth.

“Cancer Vaccines” are substances that are designed to stimulate the immune system to launch an immune response against a specific target associated with a cancer. For a general overview on immunotherapy and vaccines for cancers, see Old L. J., Scientific American, September, 1996.

Cancer vaccines may be preventative or therapeutic. Typically, preventative vaccines (for example, the flu vaccine) generally contain parts of polypeptides that stimulate the immune system to generate cells and/or other substances (for example, antibodies) that fight the target of the vaccines. Preventative vaccines must be given before exposure to the target (for example, the flu virus) in order to provide the immune system with enough time to activate and make the immune cells and substances that can attack the target. Preventative vaccines stimulate an immune response that can last for years or even an individual's lifetime.

Therapeutic vaccines are used to combat existing disease. Thus, the goal of a therapeutic cancer vaccine is not just to prevent disease, but rather to stimulate the immune system to attack existing cancerous cells. Because of the many types of cancers and because it is often unpredictable who might get cancer, among other reasons, the cancer vaccines currently being developed are therapeutic. As discussed further below, due to the difficulties associated with fighting an established cancer, most vaccines are used in combination with cytokines or adjuvants that help stimulate the immune response and/or are used in conjunction with conventional cancer therapies.

The immune system must be able to tolerate normal cells and to recognize and attack abnormal cells. To the immune system, a cancer cell may be different in very small ways from a normal cell. Therefore, the immune system often tolerates cancer cells rather than attacking them, which allows the cancer to grow and spread. Therefore, cancer vaccines must not only provoke an immune response, but also stimulate the immune system strongly enough to overcome this tolerance. The most effective anti-tumor immune responses are achieved by stimulating T cells, which can recognize and kill tumor cells directly. Therefore, most current cancer vaccines try to activate T cells directly, try to enlist antigen presenting cells (APCs) to activate T cells, or both. By way of example, researchers are attempting to enhance T cell activation by altering tumor cells so molecules that are normally only on APCs are now on the tumor cell, thus enabling the molecules to give T cells a stronger activating signal than the original tumor cells, and by evaluating cytokines and adjuvants to determine which are best at calling APCs to areas they are needed.

Cancer vaccines can be made from whole tumor cells or from substances contained by the tumor (for example, antigens). For a whole cell vaccine, tumor cells are removed from a patient(s), grown in the laboratory, and treated to ensure that they can no longer multiply and are incapable of infecting the patient. When whole tumor cells are injected into a person, an immune response against the antigens on the tumor cells is generated. There are two types of whole cell cancer vaccines: 1) autologous whole cell vaccines made with a patient's own whole, inactivated tumor cells; and 2) allogenic whole cell vaccines made with another individual's whole, inactivated tumor cells (or the tumor cells from several individuals). Antigen vaccines are not made of whole cells, but of one or more antigens contained by the tumor. Some antigens are common to all cancers of a particular type, while some are unique to an individual. A few antigens are shared between tumors of different types of cancer.

Antigens in an antigen vaccine may be delivered in several ways. For example, proteins or fragments thereof from the tumor cells can be given directly as the vaccine. Nucleic acids coding for those proteins can be given (for example, RNA or DNA vaccines). Furthermore, viral vectors can be engineered so that when they infect a human cell and the cell will make and display the tumor antigen on its surface. The viral vector should be capable of infecting only a small number of human cells in order to start an immune response, but not enough to make a person sick. Viruses can also be engineered to make cytokines or to display proteins on their surface that help activate immune cells. These can be given alone or with a vaccine to help the immune response. Finally, antibodies themselves may be used as antigens in a vaccine (anti-idiotype vaccines). In this way, an antibody to a tumor antigen is administered, then the B cells make antibodies to that antibody that also recognize the tumor cells.

Cancer vaccines frequently contain components to help boost the immune response. Cytokines (for example, IL-2), chemical messengers that recruit other immune cells to the site of attack and help killer T cells perform their function, are frequently employed. Similarly, adjuvants, substances derived from a wide variety of sources, including bacteria, have been shown to elicit immune cells to an area where they are needed. In some cases, cytokines and adjuvants are added to the cancer vaccine mixture, in other cases they are given separately.

Cancer vaccines are most frequently developed to target tumor antigens normally expressed on the cell surface (for example, membrane-bound receptors or subparts thereof). However, cancer vaccines may also be effective against intracellular antigens that are, in a tumor-specific manner, exposed on the cell surface. Many tumor antigens are intracellular proteins that are degraded and expressed on the cell surface complexed with, for example, HLA. Frequently, it is difficult to attack these antigens with antibody therapy because they are sparsely dispersed on the cell surface. However, cancer vaccines are a viable alternative therapeutic approach.

Cancer vaccines may prove most useful in preventing cancer recurrence after surgery, radiation or chemotherapy has reduced or eliminated the primary tumor.

The term “immunoassay” is an assay that utilizes the binding interaction between an antibody and an antigen. Typically, an immunoassay uses the specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at a level at least two times the background and do not substantially bind in a significant amount to other proteins present in the sample. Specific binding to an antibody under such conditions may require an antibody that is selected for its specificity for a particular protein. For example, antibodies raised to a particular Galanin, GALR2, GALR3 peptide, or GALR2-GALR3 heterocomplex polypeptide can be selected to obtain only those antibodies that are specifically immunoreactive with the Galanin, GALR2, GALR3, or GALR2-GALR3 heterocomplex polypeptide, respectively, and not with other proteins, except for polymorphic variants, orthologs, and alleles of the specific Galanin, GALR2, GALR3, or GALR2-GALR3 heterocomplex polypeptide. In addition, antibodies raised to a particular Galanin, GALR2, GALR3, or GALR2-GALR3 heterocomplex polypeptide ortholog can be selected to obtain only those antibodies that are specifically immunoreactive with the Galanin, GALR2, GALR3, or GALR2-GALR3 heterocomplex polypeptide ortholog, respectively, and not with other orthologous proteins, except for polymorphic variants, mutants, and alleles of the Galanin, GALR2, GALR3, or GALR2-GALR3 heterocomplex polypeptide ortholog. This selection may be achieved by subtracting out antibodies that cross-react with desired Galanin, GALR2, GALR3, or GALR2-GALR3 heterocomplex molecules, as appropriate. A variety of immunoassay formats may be used to select antibodies specifically immunoreactive with a particular protein. For example, solid-phase ELISA immunoassays are routinely used to select antibodies specifically immunoreactive with a protein. See, for example, Harlow & Lane, Antibodies, A Laboratory Manual, 1988, for a description of immunoassay formats and conditions that can be used to determine specific immunoreactivity.

The phrase “selectively associates with” refers to the ability of a nucleic acid to “selectively hybridize” with another as defined supra, or the ability of an antibody to “selectively (or specifically) bind” to a protein, as defined supra.

“siRNA” refers to small interfering RNAs, which also include short hairpin RNA (shRNA) (Paddison et al., Genes & Dev. 16: 948-958, 2002), that are capable of causing interference and can cause post-transcriptional silencing of specific genes in cells, for example, mammalian cells (including human cells) and in the body, for example, mammalian bodies (including humans). The phenomenon of RNA interference is described and discussed in Bass, Nature, 411:428-29, 2001; Elbashir et al., Nature, 411:494-98, 2001; and Fire et al., Nature, 391:806-11, 1998, wherein methods of making interfering RNA also are discussed. The siRNAs based upon the sequence disclosed herein (for example, GenBank Accession Number NM_{—}003614 for GALR3 mRNA sequence) is typically less than 100 base pairs (“bps”) in length and constituency and preferably is about 30 bps or shorter, and can be made by approaches known in the art, including the use of complementary DNA strands or synthetic approaches. The siRNAs are capable of causing interference and can cause post-transcriptional silencing of specific genes in cells, such as mammalian cells (including human cells) and in the body, such as mammalian bodies (including humans). Exemplary siRNAs according to the invention could have up to 29 bps, 25 bps, 22 bps, 21 bps, 20 bps, 15 bps, 10 bps, 5 bps or any number thereabout or therebetween.

The term “transgene” refers to a nucleic acid sequence encoding, for example, one of the Galanin, GALR2 or GALR3 polypeptides, or an antisense transcript thereto, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (for example, it is inserted at a location which differs from that of the natural gene or its insertion results in a knockout). A transgene can include one or more transcriptional regulatory sequences and any other nucleic acid, (for example, an intron), that may be necessary for optimal expression of a selected nucleic acid.

A “transgenic animal” refers to any animal, preferably a non-human mammal, that is chimeric, and is achievable with most vertebrate species. Such species include, but are not limited to, non-human mammals, including rodents, for example, mice and rats; rabbits; birds or amphibians; ovines, for example, sheep and goats; porcines, for example, pigs; and bovines, for example, cattle and buffalo; in which one or more of the cells of the animal contains heterologous nucleic acid introduced by way of human intervention, for example, by transgenic techniques well known in the art. The nucleic acid is introduced into the cell, directly or indirectly by introduction into a precursor of the cell, by way of deliberate genetic manipulation, for example, by microinjection or by infection with a recombinant virus. The term genetic manipulation does not include classical cross-breeding, or sexual fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the Galarun, GALR2, or GALR3 proteins, for example, either agonistic or antagonistic forms. However, transgenic animals in which the recombinant Galanin, GALR2, or GALR3 gene is silent are also contemplated. Moreover, “transgenic animal” also includes those recombinant animals in which gene disruption of one or more Galanin, GALR2, or GALR3 genes is caused by human intervention, including both recombination and antisense techniques. The transgene can be limited to somatic cells or be placed into the germline.

Methods of obtaining transgenic animals are described in, for example, Puhler, A., Ed., Genetic Engineering of Animals, VCH Pub., 1993; Murphy and Carter, Eds., Transgenesis Techniques: Principles and Protocols (Methods in Molecular Biology, Vol. 18), 1993; and Pinkert, C A., Ed., Transgenic Animal Technology: A Laboratory Handbook, Academic Press, 1994.

The term “knockout construct” refers to a nucleotide sequence that is designed to decrease or suppress expression of a polypeptide encoded by an endogenous gene in one or more cells of a mammal. The nucleotide sequence used as the knockout construct is typically comprised of (1) DNA from some portion of the endogenous gene (one or more exon sequences, intron sequences, and/or promoter sequences) to be suppressed and (2) a marker sequence used to detect the presence of the knockout construct in the cell. The knockout construct can be inserted into a cell containing the endogenous gene to be knocked out. The knockout construct can then integrate with one or both alleles of an endogenous gene, for example, Galanin, GALR2, or GALR3 gene, and such integration of the knockout construct can prevent or interrupt transcription of the full-length endogenous gene. Integration of the knockout construct into the cellular chromosomal DNA is typically accomplished via homologous recombination (i.e., regions of the knockout construct that are homologous or complementary to endogenous DNA sequences can hybridize to each other when the knockout construct is inserted into the cell; these regions can then recombine so that the knockout construct is incorporated into the corresponding position of the endogenous DNA).

By “transgenic” is meant any mammal that includes a nucleic acid sequence, which is inserted into a cell and becomes a part of the genome of the animal that develops from that cell. Such a transgene may be partly or entirely heterologous to the transgenic animal.

Thus, for example, substitution of the naturally occurring Galanin, GALR2, or GALR3 gene for a gene from a second species results in an animal that produces the receptor of the second species. Substitution of the naturally occurring gene for a gene having a mutation results in an animal that produces the mutated receptor. A transgenic mouse carrying the human Galanin, GALR2, or GALR3 receptor can be generated by direct replacement of the mouse Galanin, GALR2, or GALR3 subunit with the human gene. These transgenic animals can be critical for drug antagonist studies on animal models for human diseases, and for eventual treatment of disorders or diseases associated with the respective genes. Transgenic mice carrying these mutations will be extremely useful in studying this disease.

A transgenic animal carrying a “knockout” of Galanin, GALR2, or GALR3 gene, would be useful for the establishment of a non-human model for diseases involving such receptors, and to distinguish between the activities of the different Galanin, GALR2, or GALR3 receptors in an in vivo system. “Knockout mice” refers to mice whose native or endogenous Galanin, GALR2, or GALR3 allele or alleles have been disrupted by homologous recombination and which produce no functional Galanin, GALR2, GALR3, or GALR2-GALR3 heterocomplex of their own. Knockout mice may be produced in accordance with techniques known in the art, for example, Thomas, et al., Immunol, 163:978-84, 1999; Kanakaraj, et al., J Exp Med, 187:2073-9, 1998; or Yeh et al., Immunity, 7:715-725, 1997.

GALR3:

Human Galanin receptor 3 (GALR3) (GenBank Accession Number NM_{—}003614. Unigene clusters for GALR3 is Hs. 158353, containing 2 ESTs with Accession Number BF116239 and BF512731) appears to be at the epicenter of an amplicon that is amplified in 25% of human lung tumors and 50% of human breast tumors, implying that the gene has an important biological function so that increased DNA copies of the gene are selected for during tumor formation. GALR3 gene is also found frequently overexpressed in human lung cancers.

Initial efforts in searching for amplified regions in the genome of human lung tumors were conducted by cDNA Microarray hybridization methods. See, for example, U.S. Pat. No. 6,232,068; Pollack et al., Nat. Genet. 23(1):41-46, (1999) and other methods known in the art. In microarray analysis, EST W69399 at chromosome 22 long armband -q13- was then discovered to be amplified in lung tumor sample LU84. Further analysis of the region containing W69399 provided evidence that GALR3 is at the epicenter of an amplification region.

GALR2:

In addition to the above mentioned invention of GALR3 being frequently amplified in human cancers, the inventors studied the other 2 known galanin receptors, i.e. GALR2 and GALRI, which share 58% and 36% amino acid identity with GALR3, respectively. It was found that only GALR2 is amplified in the same lung tumors that also contain increased GALR3 gene copy number (See Tables 1 and 2). It was also found that GALR2 is amplified in the same breast tumors containing increased GALR3 gene copy number (see Table 3). DNA copy number was determined using the quantitative PCR method.

Galanin:

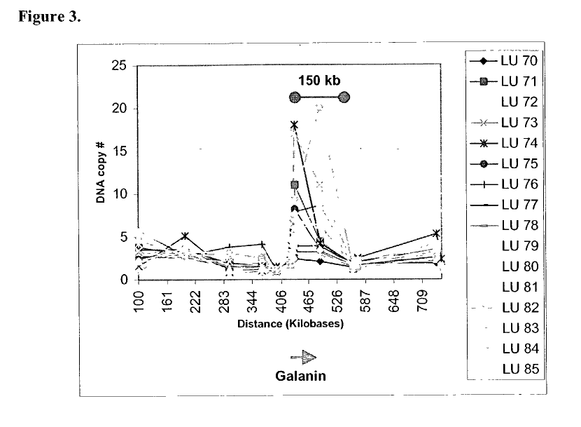

Galanin is a ligand to the Galanin receptors (GenBank Accession Number A28025, human preprogalanin cDNA sequence, [Unigene clusters for Galanin is: Hs.1907, Human Galanin.]; GenBank Accession Number CAA01907, amino acid sequence of human preprogalanin; and GenBank Accession Number AAB20740, human galanin peptide sequence). Galanin is frequently amplified (in over 70%, 54/77 samples tested) and overexpressed (in 78%, 39/50 samples tested) in the lung tumors containing amplified and overexpressed galanin receptors (See Table 7), indicating that the gene has an important biological function so that increased DNA copies of the gene are selected for during tumor formation. Galanin appears to be at the epicenter of an amplicon (See FIG. 3) that is amplified in over 70% of human lung tumors. The Galanin gene and its expressed protein product can thus be used diagnostically or as targets for cancer therapy; and they can also be used to identify compounds useful in the diagnosis, prevention, and therapy of tumors and cancers (for example, lung cancer).

Amplification of GALR2 and GALR3 Genes in Tumors:

The presence of a target gene that has undergone amplification in tumors is evaluated by determining the copy number of the target genes, i.e., the number of DNA sequences in a cell encoding the target protein. Generally, a normal diploid cell has two copies of a given autosomal gene. The copy number can be increased, however, by gene amplification or duplication, for example, in cancer cells, or reduced by deletion. Methods of evaluating the copy number of a particular gene are well known in the art, and include, inter alia, hybridization and amplification based assays.

TABLE 1
Co-amplification GALR2-GALR3 in lung tumors.

| | DNA Copy Number | | |
|---|---|---|---|
| Lung Tumors | GALR3 (WA15) | GALR2 | GALR1 |
| LU70 | 2.7 | 2.2 | 0.93 |
| LU71 | 7.4 | 6.4 | 0.52 |
| LU72 | 3.3 | 2.5 | 1.3 |
| LU73 | 32 | 17 | 0.89 |
| LU74 | 10 | 8.1 | 1.1 |
| LU75 | 5.8 | 6.9 | 0.88 |
| LU76 | 4.1 | 3.5 | 0.96 |
| LU77 | 1.5 | 3.1 | 0.99 |
| LU78 | 5.1 | 2.4 | 1.2 |
| LU79 | 7 | 4.2 | 1.1 |
| LU80 | 4.9 | 3.6 | 0.7 |
| LU81 | 6.5 | 3.3 | 1.1 |
| LU82 | 11 | 7.8 | 0.97 |
| LU83 | 26 | 12 | 1.1 |
| LU84 | 58 | 3.4 | 0.41 |
| LU85 | 6.8 | 3.7 | 1.3 |

TABLE 2
Co-amplification GALR2-GALR3 in lung tumors.

DNA Copy Number

| Lung Tumors | GALR2 | GALR3 |
|---|---|---|
| LU70 | 2 | 14 |
| LU71 | 9.4 | 13 |
| LU72 | 2.9 | 3.9 |
| LU73 | 16 | 24 |
| LU74 | 9.3 | 16 |
| LU75 | 8.2 | 6.8 |
| LU76 | 3.9 | 5 |
| LU77 | 3.4 | 2 |
| LU78 | 2.7 | 3.3 |
| LU79 | 4.6 | 10 |
| LU80 | 5.3 | 6.8 |
| LU81 | 3.8 | 6.8 |
| LU82 | 4.9 | 9.4 |
| LU83 | 9.6 | 31 |
| LU84 | 2.7 | 47 |
| LU85 | 4.6 | 5.7 |

TABLE 3
Co-amplification GALR2-GALR3 in breast tumors.

DNA Copy Number

| Breast Tumors | GALR2 | GALR3 |
|---|---|---|
| BR17 | 5.3 | 15 |
| BR18 | 2.0 | 4.2 |
| BR19 | 2.6 | 10 |
| BR20 | 3.8 | 12 |
| BR21 | 3.7 | 17 |
| BR22 | 9.8 | 19 |
| BR23 | 17 | 27 |
| BR24 | 3.9 | 11 |
| BR49 | 3.8 | 3.4 |
| BR50 | 4.5 | 3.0 |
| BR51 | 4.2 | 7.5 |
| BR52 | 3.8 | 3.4 |
| BR53 | 3.1 | 13 |
| BR54 | 7.8 | 20 |
| BR55 | 4.9 | 4.8 |
| BR56 | 8.0 | 10 |

Any of a number of hybridization based assays can be used to detect the copy number of the Galanin, GALR2, or GALR3 gene in the cells of a biological sample. One such method is Southern blot (see Ausubel et al., or Sambrook et al., supra), where the genomic DNA is typically fragmented, separated electrophoretically, transferred to a membrane, and subsequently hybridized to a Galanin, GALR2 or GALR3 specific probe. Comparison of the intensity of the hybridization signal from the probe for the target region with a signal from a control probe from a region of normal nonamplified, single-copied genomic DNA in the same genome provides an estimate of the relative Galanin, GALR2 or GALR3 copy number, corresponding to the specific probe used. An increased signal compared to control represents the presence of amplification.

A methodology for determining the copy number of the Galanin, GALR2 or GALR3 gene in a sample is in situ hybridization, for example, fluorescence in situ hybridization (FISH) (see Angerer, 1987 Meth. Enzymol., 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

Another alternative methodology for determining number of DNA copies is comparative genomic hybridization (CGH). In comparative genomic hybridization methods, a “test” collection of nucleic acids is labeled with a first label, while a second collection (for example, from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection, is detected and the ratio provides a measure of the Galanin, GALR2 or GALR3 gene copy number, corresponding to the specific probe used. A cytogenetic representation of DNA copy-number variation can be generated by CGH, which provides fluorescence ratios along the length of chromosomes from differentially labeled test and reference genomic DNAs.

Hybridization protocols suitable for use with the methods of the invention are described, for example, in Albertson (1984) EMBO J 3:1227-1234; Pinkel (1988) Proc. Natl. Acad. Sci. USA, 85:9138-9142; EPO Pub. No. 430:402; Methods in Molecular Biology, Vol. 33: In Situ Hybridization Protocols, Choo, ed., Humana Press, Totowa, N.J. (1994).

Amplification-based assays also can be used to measure the copy number of the Galanin, GALR2, or GALR3 gene. In such assays, the corresponding Galanin, GALR2, or GALR3 nucleic acid sequences act as a template in an amplification reaction (for example, Polymerase Chain Reaction or PCR). In a quantitative amplification, the amount of amplification product will be proportional to the amount of template in the original sample. Comparison to appropriate controls provides a measure of the copy number of the Galanin, GALR2, or GALR3 gene, corresponding to the specific probe used, according to the principle discussed above. Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

A Taqman-based assay can also be used to quantify Galanin, GALR2, or GALR3 polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

Other suitable amplification methods include, but are not limited to, ligase chain reaction (LCR) (see, Wu and Wallace, Genomics, 4: 560, 1989; Landegren et al., Science, 241: 1077, 1988; and Barringer et al, Gene, 89:117, 1990), transcription amplification (Kwoh et al., Proc. Natl. Acad. Sci. USA, 86:1173, 1989), self-sustained sequence replication (Guatelli et al., Proc Nat Acad Sci, USA 87:1874, 1990), dot PCR, and linker adapter PCR, etc.

One powerful method for determining DNA copy numbers uses microarray-based platforms. Microarray technology may be used because it offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068; Pollack et al., Nat. Genet., 23(1):41-6 (1999), and others.

As demonstrated in the Examples set forth herein, the Galanin, GALR2, or GALR3 gene is frequently amplified in certain cancers, particularly breast cancers; and it resides at the epicenter of the amplified chromosome region. All samples showing Galanin, GALR2 and GALR3 genes amplification in Table 5 and Table 7 also demonstrate overexpression of Galanin, GALR2, and GALR3 mRNA. The Galanin, GALR2 and GALR3 genes has these characteristic features of overexpression, amplification, and the correlation between the two, and these features are shared with other well studied oncogenes (Yoshimoto et al., JPN J Cancer Res, 77(6):540-5, 1986; Knuutila et al., Am. J. Pathol., 152(5):1107-23, 1998). The Galanin, GALR2, and GALR3 genes are accordingly used in the present invention as a target for cancer diagnosis and treatment.

Frequent Overexpression of Galanin, GALR2, and GALR3 Genes in Tumors:

The expression levels of the Galanin, GALR2, or GALR3 gene in tumors cells were examined. As demonstrated in the examples infra, GALR3 gene is overexpressed in lung, brain, breast, colon, prostate, and ovarian cancers. Detection and quantification of the Galanin, GALR2, or GALR3 gene expression may be carried out through direct hybridization based assays or amplification based assays. The hybridization based techniques for measuring gene transcript are known to those skilled in the art (Sambrook et al., Molecular Cloning: A Laboratory Manual, 2d Ed. vol. 1-3, Cold Spring Harbor Press, NY, 1989). For example, one method for evaluating the presence, absence, or quantity of the Galanin, GALR2 or GALR3 gene is by Northern blot. Isolated mRNAs from a given biological sample are electrophoresed to separate the mRNA species, and transferred from the gel to a membrane such as a nitrocellulose or nylon filter. Labeled Galanin, GALR2 or GALR3 probes are then hybridized to the membrane to identify and quantify the respective mRNAs. The example of amplification based assays include RT-PCR, which is well known in the art (Ausubel et al., Current Protocols in Molecular Biology, eds. 1995 supplement). Quantitative RT-PCR is used preferably to allow the numerical comparison of the level of respective Galanin, GALR2 or GALR3 mRNAs in different samples.

### Cancer Diagnosis, Therapies, And Vaccines Using Galanin, GALR2, GALR3, and GALR2-GALR3 Heterocomplex:

A. Overexpression and Amplification of the Galanin, GALR2, and GALR3 Genes:

The Galanin, GALR2, and GALR3 genes and their expressed protein product can be used for diagnosis, prognosis, rational drug design, and other therapeutic intervention of tumors and cancers (for example, a lung cancer, a brain cancer, a breast cancer, a colon cancer, a prostate cancer, or an ovarian cancer).

Detection and measurement of amplification and/or overexpression of the Galanin, GALR2, or GALR3 gene in a biological sample taken from a patient indicates that the patient may have developed a tumor. Particularly, the presence of amplified Galanin, GALR2, or GALR3 DNA leads to a diagnosis of cancer or precancerous condition, for example, a lung cancer, a brain cancer, a breast cancer, a colon cancer, a prostate cancer, or an ovarian cancer, respectively, with high probability of accuracy. The present invention therefore provides, in one aspect, methods for diagnosing or characterizing a cancer or tumor in a mammalian tissue by measuring the levels of Galanin, GALR2, or GALR3 mRNA expression in samples taken from the tissue of suspicion, and determining whether Galanin, GALR2, or GALR3 is overexpressed in the tissue. The various techniques, including hybridization based and amplification based methods, for measuring and evaluating mRNA levels are provided herein as discussed supra. The present invention also provides, in another aspect, methods for diagnosing a cancer or tumor in a mammalian tissue by measuring the numbers of Galanin, GALR2, or GALR3 DNA copy in samples taken from the tissue of suspicion, and determining whether the Galanin, GALR2, or GALR3 gene is amplified in the tissue. The various techniques, including hybridization based and amplification based methods, for measuring and evaluating DNA copy numbers are provided herein as discussed supra. The present invention thus provides methods for detecting amplified genes at DNA level and increased expression at RNA level; wherein both the results are indicative of tumor progression.

B. Detection of the Galanin Protein, GALR2 Protein, GALR3 Protein, or GALR2-GALR3 Heterocomplex Protein:

According to the present invention, the detection of increased Galanin protein, GALR2 protein, GALR3 protein, or GALR2-GALR3 heterocomplex protein level in a biological subject may also suggest the presence of a precancerous or cancerous condition in the tissue source of the sample. Protein detection for tumor and cancer diagnostics and prognostics can be carried out by immunoassays, for example, using antibodies directed against a target such as Galanin protein, GALR2 protein, GALR3 protein, or GALR2-GALR3 heterocomplex protein. Any methods that are known in the art for protein detection and quantitation can be used in the methods of this invention, including, inter alia, electrophoresis, capillary electrophoresis, high performance liquid chromatography (HPLC), thin layer chromatography (TLC), hyperdiffusion chromatography, immunoelectrophoresis, radioimmunoassay (RIA), enzyme-linked immunosorbent assays (ELISAs), immunoflouorescent assays, Western Blot, etc. Protein from the tissue or cell type to be analyzed may be isolated using standard techniques, for example, as described in Harlow and Lane, Antibodies: A Laboratory Manual (Cold Spring Harbor Laboratory Press, Cold Spring Harbor, N.Y. 1988).

The antibodies (or fragments thereof) useful in the present invention can, additionally, be employed histologically, as in immunofluorescence or immunoelectron microscopy, for in situ detection of target gene peptides. In situ detection can be accomplished by removing a histological specimen from a patient, and applying thereto a labeled antibody of the present invention. The antibody (or its fragment) is preferably applied by overlaying the labeled antibody (or fragment) onto a biological sample. Through the use of such a procedure, it is possible to determine not only the presence of the target gene product such as Galanin protein, GALR2 protein, GALR3 protein, or GALR2-GALR3 heterocomplex protein, but also their distribution in the examined tissue. Using the present invention, a skilled artisan will readily perceive that any of a wide variety of histological methods (such as staining procedures) can be modified to achieve such in situ detection.

The biological sample that is subjected to protein detection can be brought in contact with and immobilized on a solid phase support or carrier such as nitrocellulose, or other solid support which is capable of immobilizing cells, cell particles, or soluble proteins. The support can then be washed with suitable buffers followed by treatment with the detectably labeled fingerprint gene specific antibody. The solid phase support can then be washed with the buffer a second time to remove unbound antibody. The amount of bound label on the solid support can then be detected by conventional means.

A target product-specific antibody such as a Galanin, GALR2, GALR3, or GALR2-GALR3 heterocomplex antibody can be detectably labeled, in one aspect, by linking the same to an enzyme, for example, horseradish peroxidase, alkaline phosphatase, or glucoamylase, and using it in an enzyme immunoassay (EIA) (see, for example, Voller, A., 1978, The Enzyme Linked Immunosorbent Assay (ELISA), Diagnostic Horizons, 2:1-7; Voller et al., J. Clin. Pathol., 31:507-520, 1978; Butler, J. E., Meth. Enzymol., 73:482-523, 1981; Maggio, E. (ed.), Enzyme Immunoassay, CRC Press, Boca Raton, Fla., 1980; and Ishikawa et al. (eds), Enzyme Immunoassay, Kgaku Shoin, Tokyo, 1981). The enzyme bound to the antibody reacts with an appropriate substrate, preferably a chromogenic substrate, in such a manner as to produce a chemical moiety that can be detected, for example, by spectrophotometric or fluorimetric means, or by visual inspection.

In a related aspect, therefore, the present invention provides the use of Galanin, GALR2, GALR3, and GALR2-GALR3 heterocomplex antibodies in cancer diagnosis and intervention. Antibodies that specifically bind to Galanin protein, GALR2 protein, GALR3 protein, or GALR2-GALR3 heterocomplex protein, and polypeptides can be produced by a variety of methods. Such antibodies may include, but are not limited to, polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above.

Such antibodies can be used, for example, in the detection of the target gene, Galanin protein, GALR2 protein, GALR3 protein, or GALR2-GALR3 heterocomplex protein, or its fingerprint or pathway genes involved in a particular biological pathway, which may be of physiological or pathological importance. The Galanin, GALR2, GALR3, or GALR2-GALR3 heterocomplex antibodies can also be used in a method for the inhibition of Galanin, GALR2, GALR3, or GALR2-GALR3 heterocomplex activity, respectively. Thus, such antibodies can be used in treating tumors and cancers (for example, lung cancer, brain cancer, breast cancer, colon cancer, prostate cancer, or ovarian cancer); they may also be used in diagnostic procedures whereby patients are tested for abnormal levels of Galanin protein, GALR2 protein, GALR3 protein, or GALR2-GALR3 heterocomplex protein, and/or fingerprint or pathway gene proteins associated with Galanin, GALR2, GALR3, or GALR2-GALR3 heterocomplex, respectively, and for the presence of abnormal forms of such proteins.

To produce antibodies to Galanin protein, GALR2 protein, GALR3 protein, or GALR2-GALR3 heterocomplex protein, a host animal is immunized with the protein, or a portion thereof. Such host animals can include, but are not limited to, rabbits, mice, and rats. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin (KLH), dinitrophenol (DNP), and potentially useful human adjuvants such as BCG (Bacille Calmette-Guerin) and Corynebacterium parvum.

Monoclonal antibodies, which are homogeneous populations of antibodies to a particular antigen, such as a Galanin protein, GALR2 protein, GALR3 protein, or GALR2-GALR3 heterocomplex protein, as in the present invention, can be obtained by any technique which provides for the production of antibody molecules by continuous cell lines in culture. These include, but are not limited to the hybridoma technique of Kohler and Milstein, (Nature, 256:495-497, 1975; and U.S. Pat. No. 4,376,110), the human B-cell hybridoma technique (Kosbor et al., Immunology Today, 4:72, 1983; Cole et al., Proc. Natl. Acad. Sci. USA, 80:2026-2030, 1983), and the BV-hybridoma technique (Cole et al., Monoclonal Antibodies And Cancer Therapy (Alan R. Liss, Inc. 1985), pp. 77-96. Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

In addition, techniques developed for the production of “chimeric antibodies” can be made by splicing the genes from a mouse antibody molecule of appropriate antigen specificity together with genes from a human antibody molecule of appropriate biological activity (see, Morrison et al., Proc. Natl. Acad. Sci. USA, 81:6851-6855, 1984; Neuberger et al., Nature, 312:604-608, 1984; Takeda et al., Nature, 314:452-454, 1985; and U.S. Pat. No. 4,816,567). A chimeric antibody is a molecule in which different portions are derived from different animal species, for example, those having a variable region derived from a murine mAb and a container region derived from human immunoglobulin.

Alternatively, techniques described for the production of single chain antibodies (for example, U.S. Pat. No. 4,946,778; Bird, Science, 242:423-426, 1988; Huston et al., Proc. Natl. Acad. Sci. USA, 85:5879-5883, 1988; and Ward et al., Nature, 334:544-546, 1989), and for making humanized monoclonal antibodies (U.S. Pat. No. 5,225,539), can be used to produce anti-differentially expressed or anti-pathway gene product antibodies.

Antibody fragments that recognize specific epitopes can be generated by known techniques. For example, such fragments include but are not limited to: the F(ab′)₂ fragments that can be produced by pepsin digestion of the antibody molecule, and the Fab fragments that can be generated by reducing the disulfide bridges of the F(ab′)₂ fragments. Alternatively, Fab expression libraries can be constructed (Huse et al., Science, 246:1275-1281, 1989) to allow rapid and easy identification of monoclonal Fab fragments with the desired specificity.

C. Use of Galanin, GALR2, GALR3, and GALR2-GALR3 Heterocomplex Modulators in Cancer Diagnostics:

In addition to antibodies, the present invention provides, in another aspect, the diagnostic and therapeutic utilities of other molecules and compounds that interact with Galanin protein, GALR2 protein, GALR3 protein, or GALR2-GALR3 heterocomplex protein. Specifically, such compounds can include, but are not limited to proteins or peptides, comprising extracellular portions of transmembrane proteins of the target, if they exist. Exemplary peptides include soluble peptides, for example, Ig-tailed fusion peptides. Such compounds can also be obtained through the generation and screening of random peptide libraries (see, for example, Lam et al., Nature, 354:82-84, 1991; Houghton et al., Nature, 354:84-86, 1991), made of D- and/or L-configuration amino acids, phosphopeptides (including, but not limited to, members of random or partially degenerate phosphopeptide libraries; see, for example, Songyang et al., Cell, 72:767-778, 1993), and small organic or inorganic molecules. In this aspect, the present invention provides a number of methods and procedures to assay or identify compounds that bind to target, i.e., Galanin protein, GALR2 protein, GALR3 protein, or GALR2-GALR3 heterocomplex protein, or to any cellular protein that may interact with the target, and compounds that may interfere with the interaction of the target with other cellular proteins.

In vitro assay systems are provided that are capable of identifying compounds that specifically bind to the target gene product, for example, Galanin protein, GALR2 protein, GALR3 protein, or GALR2-GALR3 heterocomplex protein. The assays all involve the preparation of a reaction mixture of the target gene product such as Galanin protein, GALR2 protein, GALR3 protein, or GALR2-GALR3 heterocomplex protein and a test compound under conditions and for a time sufficient to allow the two components to interact and bind, thus forming a complex that can be removed and/or detected in the reaction mixture. These assays can be conducted in a variety of ways. For example, one method involves anchoring the target protein or the test substance to a solid phase, and detecting target protein—test compound complexes anchored to the solid phase at the end of the reaction. In one aspect of such a method, the target protein can be anchored onto a solid surface, and the test compound, which is not anchored, can be labeled, either directly or indirectly. In practice, microtiter plates can be used as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

To conduct the assay, the non-immobilized component is added to the coated surface containing the anchored component. After the reaction is complete, unreacted components are removed, for example, by washing, and complexes anchored on the solid surface are detected. Where the previously immobilized component is pre-labeled, the detection of label immobilized on the surface indicates that complexes were formed. Where the previously non-immobilized component is not pre-labeled, an indirect label can be used to detect complexes anchored on the surface; for example, using a labeled antibody specific for the immobilized component (the antibody, in turn, can be directly labeled or indirectly labeled with a labeled anti-Ig antibody). Alternatively, the reaction can be conducted in a liquid phase, the reaction products separated from unreacted components, and complexes detected, for example, using an immobilized antibody specific for a target gene or the test compound to anchor any complexes formed in solution, and a labeled antibody specific for the other component of the possible complex to detect anchored complexes.

Assays are also provided for identifying any cellular protein that may interact with the target protein, i.e., Galanin, GALR2, GALR3, or GALR2-GALR3 heterocomplex protein. Any method suitable for detecting protein-protein interactions can be used to identify novel interactions between target protein and cellular or extracellular proteins. Those cellular or extracellular proteins may be involved in certain cancers, for example, lung cancer, brain cancer, breast cancer, colon cancer, prostate cancer, or ovarian cancer, and represent certain tumorigenic pathways including the target, for example, Galanin, GALR2, or GALR3. They may thus be denoted as pathway genes.

Methods such as co-immunoprecipitation and co-purification through gradients or chromatographic columns can be used to identify protein-protein interactions engaged by the target protein. The amino acid sequence of the target protein, i.e., Galanin, GALR2, or GALR3 protein or a portion thereof, is useful in identifying the pathway gene products or other proteins that interact with Galanin, GALR2, or GALR3 protein. The amino acid sequence can be derived from the nucleotide sequence, or from published database records (SWISS-PROT, PIR, EMBL); it can also be ascertained using techniques well known to a skilled artisan, for example, the Edman degradation technique (see, for example, Creighton, Proteins: Structures and Molecular Principles, 1983, W. H. Freeman & Co., N.Y., 34-49). The nucleotide subsequences of the target gene, for example, Galanin, GALR2, or GALR3, can be used in a reaction mixture to screen for pathway gene sequences. Screening can be accomplished, for example, by standard hybridization or PCR techniques. Techniques for the generation of oligonucleotide mixtures and the screening are well known (see, for example, Ausubel, supra, and Innis et al. (eds.), PCR Protocols: A Guide to Methods and Applications, 1990, Academic Press, Inc., New York).

By way of example, the yeast two-hybrid system which is often used in detecting protein interactions in vivo is discussed herein. Chien et al. has reported the use of a version of the yeast two-hybrid system (Proc. Natl. Acad. Sci. USA, 1991, 88:9578-9582); it is commercially available from Clontech (Palo Alto, Calif.). Briefly, utilizing such a system, plasmids are constructed that encode two hybrid proteins: the first hybrid protein comprises the DNA-binding domain of a transcription factor, for example, activation protein, fused to a known protein, in this case, a protein known to be involved in a tumor or cancer, and the second hybrid protein comprises the transcription factor's activation domain fused to an unknown protein that is encoded by a cDNA which has been recombined into this plasmid as part of a cDNA library. The plasmids are transformed into a strain of the yeast Saccharomyces cerevisiae that contains a reporter gene, for example, lacZ, whose expression is regulated by the transcription factor's binding site. Either hybrid protein alone cannot activate transcription of the reporter gene. The DNA binding hybrid protein cannot activate transcription because it does not provide the activation domain function, and the activation domain hybrid protein cannot activate transcription because it lacks the domain required for binding to its target site, i.e., it cannot localize to the transcription activator protein's binding site. Interaction between the DNA binding hybrid protein and the library encoded protein reconstitutes the functional transcription factor and results in expression of the reporter gene, which is detected by an assay for the reporter gene product

The two-hybrid system or similar methods can be used to screen activation domain libraries for proteins that interact with a known “bait” gene product. The Galanin, GALR2, or GALR3 gene product, involved in a number of tumors and cancers, is such a bait according to the present invention. Total genomic or cDNA sequences are fused to the DNA encoding an activation domain. This library and a plasmid encoding a hybrid of the bait gene product, i.e., Galanin, GALR2, or GALR3 protein or polypeptides, fused to the DNA-binding domain are co-transformed into a yeast reporter strain, and the resulting transformants are screened for those that express the reporter gene. For example, the bait gene Galanin, GALR2, or GALR3 can be cloned into a vector such that it is translationally fused to the DNA encoding the DNA-binding domain of the GAL4 protein. The colonies are purified and the (library) plasmids responsible for reporter gene expression are isolated. The inserts in the plasmids are sequenced to identify the proteins encoded by the cDNA or genomic DNA.

A cDNA library of a cell or tissue source that expresses proteins predicted to interact with the bait gene product, for example, Galanin, GALR2, or GALR3, can be made using methods routinely practiced in the art. According to the particular system described herein, the library is generated by inserting the cDNA fragments into a vector such that they are translationally fused to the activation domain of GAL4. This library can be cotransformed along with the bait gene-GAL4 fusion plasmid into a yeast strain which contains a lacZ gene whose expression is controlled by a promoter which contains a GAL4 activation sequence. A cDNA encoded protein, fused to GAL4 activation domain, that interacts with the bait gene product will reconstitute an active GAL4 transcription factor and thereby drive expression of the lacZ gene. Colonies that express lacZ can be detected by their blue color in the presence of X-gal. cDNA containing plasmids from such a blue colony can then be purified and used to produce and isolate the Galanin, GALR2, or GALR3-interacting protein using techniques routinely practiced in the art.

In another aspect, the present invention also provides assays for compounds that interfere with gene and cellular protein interactions involving the target Galanin protein, GALR2 protein, GALR3 protein, or GALR2-GALR3 heterocomplex protein. The target gene product such as Galanin protein, GALR2 protein, GALR3 protein, or GALR2-GALR3 heterocomplex protein may interact in vivo with one or more cellular or extracellular macromolecules, such as proteins and nucleic acid molecules. Such cellular and extracellular macromolecules are referred to as “binding partners.” Compounds that disrupt such interactions can be used to regulate the activity of the target gene product such as Galanin protein, GALR2 protein, GALR3 protein, or GALR2-GALR3 heterocomplex protein, especially mutant target gene product. Such compounds can include, but are not limited to, molecules such as antibodies, peptides and other chemical compounds.

The assay systems all involve the preparation of a reaction mixture containing the target gene product Galanin protein, GALR2 protein, GALR3 protein, or GALR2-GALR3 heterocomplex protein, and the binding partner under conditions and for a time sufficient to allow the two products to interact and bind, thus forming a complex. To test a compound for inhibitory activity, the reaction mixture is prepared in the presence and absence of the test compound. The test compound can be initially included in the reaction mixture, or can be added at a time subsequent to the addition of a target gene product and its cellular or extracellular binding partner. Control reaction mixtures are incubated without the test compound or with a placebo. The formation of complexes between the target gene product Galanin protein, GALR2 protein, GALR3 protein, or GALR2-GALR3 heterocomplex protein and the cellular or extracellular binding partner is then detected. The formation of a complex in the control reaction, but not in the reaction mixture containing the test compound, indicates that the compound interferes with the interaction of the target gene product Galanin protein, GALR2 protein, GALR3 protein, or GALR2-GALR3 heterocomplex protein and the interactive binding partner. Additionally, complex formation within reaction mixtures containing the test compound and normal target gene product can be compared to complex formation within reaction mixtures containing the test compound and mutant target gene product. This comparison can be important in the situation where it is desirable to identify compounds that disrupt interactions of mutant but not normal target gene product.

The assays can be conducted in a heterogeneous or homogeneous format. Heterogeneous assays involve anchoring either the target gene product Galanin protein, GALR2 protein, GALR3 protein, or GALR2-GALR3 heterocomplex protein or the binding partner to a solid phase and detecting complexes anchored to the solid phase at the end of the reaction, as described above. In homogeneous assays, the entire reaction is carried out in a liquid phase, as described below. In either approach, the order of addition of reactants can be varied to obtain different information about the compounds being tested. For example, test compounds that interfere with the interaction between the target gene product Galanin protein, GALR2 protein, GALR3 protein, or GALR2-GALR3 heterocomplex protein and the binding partners, for example, by competition, can be identified by conducting the reaction in the presence of the test substance; i.e., by adding the test substance to the reaction mixture prior to or simultaneously with the target gene product Galanin protein, GALR2 protein, GALR3 protein, or GALR2-GALR3 heterocomplex protein and interactive cellular or extracellular binding partner. Alternatively, test compounds that disrupt preformed complexes, for example, compounds with higher binding constants that displace one of the components from the complex, can be tested by adding the test compound to the reaction mixture after complexes have been formed.

In a homogeneous assay, a preformed complex of the target gene product and the interactive cellular or extracellular binding partner product is prepared in which either the target gene products or their binding partners are labeled, but the signal generated by the label is quenched due to complex formation (see, for example, Rubenstein, U.S. Pat. No. 4,109,496). The addition of a test substance that competes with and displaces one of the species from the preformed complex will result in the generation of a signal above background. The test substances that disrupt the interaction between the target gene product Galanin protein, GALR2 protein, GALR3 protein, or GALR2-GALR3 heterocomplex protein and cellular or extracellular binding partners can thus be identified.

In one aspect, the target gene product Galanin protein, GALR2 protein, GALR3 protein, or GALR2-GALR3 heterocomplex protein can be prepared for immobilization using recombinant DNA techniques. For example, the target Galanin, GALR2, or GALR3 gene coding region can be fused to a glutathione-S-transferase (GST) gene using a fusion vector such as pGEX-5X-1, in such a manner that its binding activity is maintained in the resulting fusion product. The interactive cellular or extracellular binding partner product is purified, and used to raise a monoclonal antibody, using methods routinely practiced in the art. This antibody can be labeled with the radioactive isotope ¹²⁵I, for example, by methods routinely practiced in the art.

In a heterogeneous assay, the GST-Target gene fusion product is anchored, for example, to glutathione-agarose beads. The interactive cellular or extracellular binding partner is then added in the presence or absence of the test compound in a manner that allows interaction and binding to occur. At the end of the reaction period, unbound material is washed away, and the labeled monoclonal antibody can be added to the system and allowed to bind to the complexed components. The interaction between the target gene product Galanin protein, GALR2 protein, GALR3 protein, or GALR2-GALR3 heterocomplex protein and the interactive cellular or extracellular binding partner is detected by measuring the corresponding amount of radioactivity that remains associated with the glutathione-agarose beads. A successful inhibition of the interaction by the test compound will result in a decrease in measured radioactivity. Alternatively, the GST-target gene fusion product and the interactive cellular or extracellular binding partner can be mixed together in liquid in the absence of the solid glutathione-agarose beads. The test compound is added either during or after the binding partners are allowed to interact. This mixture is then added to the glutathione-agarose beads and unbound material is washed away. Again, the extent of inhibition of the binding partner interaction can be detected by adding the labeled antibody and measuring the radioactivity associated with the beads.

In other aspects of the invention, these same techniques are employed using peptide fragments that correspond to the binding domains of the target gene product such as Galanin protein, GALR2 protein, GALR3 protein, or GALR2-GALR3 heterocomplex protein and the interactive cellular or extracellular binding partner (where the binding partner is a product), in place of one or both of the full-length products. Any number of methods routinely practiced in the art can be used to identify and isolate the protein's binding site. These methods include, but are not limited to, mutagenesis of one of the genes encoding one of the products and screening for disruption of binding in a co-immunoprecipitation assay.

Additionally, compensating mutations in the gene encoding the second species in the complex can be selected. Sequence analysis of the genes encoding the respective products will reveal mutations that correspond to the region of the product involved in interactive binding. Alternatively, one product can be anchored to a solid surface using methods described above, and allowed to interact with and bind to its labeled binding partner, which has been treated with a proteolytic enzyme, for example, trypsin. After washing, a short, labeled peptide comprising the binding domain can remain associated with the solid material, which can be isolated and identified by amino acid sequencing. Also, once the gene coding for the cellular or extracellular binding partner product is obtained, short gene segments can be engineered to express peptide fragments of the product, which can then be tested for binding activity and purified or synthesized.

D. Methods For Cancer Treatment Using Galanin, GALR2 and GALR3 Modulators:

In another aspect, the present invention provides methods for treating or controlling a cancer or tumor and the symptoms associated therewith. Any of the binding compounds, for example, those identified in the aforementioned assay systems, can be tested for the ability to prevent and/or ameliorate symptoms of tumors and cancers (for example, breast cancer, colon cancer, lung cancer, brain cancer, prostate cancer, or ovarian cancer). As used herein, inhibit, control, ameliorate, prevent, treat, and suppress collectively and interchangeably mean stopping or slowing cancer formation, development, or growth and eliminating or reducing cancer symptoms. Cell-based and animal model-based trial systems for evaluating the ability of the tested compounds to prevent and/or ameliorate tumors and cancers symptoms are used according to the present invention.

For example, cell based systems can be exposed to a compound suspected of ameliorating breast, colon, lung cancer, brain cancer, prostate cancer, or ovarian tumor or cancer symptoms, at a sufficient concentration and for a time sufficient to elicit such an amelioration in the exposed cells. After exposure, the cells are examined to determine whether one or more tumor or cancer phenotypes has been altered to resemble a more normal or more wild-type, non-cancerous phenotype. Further, the levels of Galanin, GALR2, or GALR3 mRNA expression and DNA amplification within these cells may be determined, according to the methods provided supra. A decrease in the observed level of expression and amplification would indicate to a certain extent the successful intervention of tumors and cancers (for example, lung cancer, brain cancer, breast cancer, colon cancer, prostate cancer, or ovarian cancer).

In addition, animal models can be used to identify compounds for use as drugs and pharmaceuticals that are capable of treating or suppressing symptoms of tumors and cancers. For example, animal models can be exposed to a test compound at a sufficient concentration and for a time sufficient to elicit such an amelioration in the exposed animals. The response of the animals to the exposure can be monitored by assessing the reversal of symptoms associated with the tumor or cancer, or by evaluating the changes in DNA copy number and levels of mRNA expression of the target gene such as Galanin, GALR2, or GALR3. Any treatments which reverse any symptom of tumors and cancers; and/or which reduce overexpression and amplification of the target Galanin, GALR2, or GALR3 gene may be considered as candidates for therapy in humans. Dosages of test agents can be determined by deriving dose-response curves.

Moreover, fingerprint patterns or gene, gene expression profiles can be characterized for known cell states, for example, normal or known pre-neoplastic, neoplastic, or metastatic states, within the cell- and/or animal-based model systems. Subsequently, these known fingerprint patterns can be compared to ascertain the ability of a test compound to modify such fingerprint patterns, and to cause the pattern to more closely resemble that of a normal fingerprint pattern. For example, administration of a compound which interacts with and affects Galanin, GALR2, or GALR3 gene expression and amplification may cause the fingerprint pattern of a cancerous model system to more closely resemble a control, normal system; such a compound thus will have therapeutic utilities in treating the cancer. In other situations, administration of a compound may cause the fingerprint pattern of a control system to begin to mimic tumors and cancers (for example, lung cancer, brain cancer, breast cancer, colon cancer, prostate cancer, or ovarian cancer); such a compound therefore acts as a tumorigenic agent, which in turn can serve as a target for therapeutic interventions of the cancer and its diagnosis.

E. Methods for Monitoring Efficacy of Cancer Treatment:

In a further aspect, the present invention provides methods for monitoring the efficacy of a therapeutic treatment regimen of cancer and methods for monitoring the efficacy of a compound in clinical trials for inhibition of tumors. The monitoring can be accomplished by detecting and measuring, in the biological samples taken from a patient at various time points during the course of the application of a treatment regimen for treating a cancer or a clinical trial, the changed levels of expression or amplification of the target gene such as Galanin, GALR2, or GALR3. A level of expression and/or amplification that is lower in samples taken at the later time of the treatment or trial then those at the earlier date indicates that the treatment regimen is effective to control the cancer in the patient, or the compound is effective in inhibiting the tumor. The time course studies should be so designed that sufficient time is allowed for the treatment regimen or the compound to exert its effect.

Therefore, the influence of compounds on tumors and cancers can be monitored both in a clinical trial and in a basic drug screening. In a clinical trial, for example, tumor cells can be isolated from lung, brain, breast, colon, prostate, or ovarian tumors removed by surgery, and RNA prepared and analyzed by Northern blot analysis or Taqman RT-PCR as described herein, or alternatively by measuring the amount of protein produced. The fingerprint expression profiles thus generated can serve as putative biomarkers for lung, brain, breast, colon, prostate, or ovarian tumors or cancers. Particularly, the expression of Galanin, GALR2, or GALR3 serves as one such biomarker. Thus, by monitoring the level of expression of the differentially or over-expressed genes such as Galanin, GALR2, or GALR3, an effective treatment protocol can be developed using suitable chemotherapeutic anticancer drugs.

F. Use of Modulators to Galanin, GALR2 or GALR3 Nucleotides in Cancer Treatment:

In a further aspect of this invention, additional compounds and methods for treatment of tumors are provided. Symptoms of tumors and cancers can be controlled by, for example, target gene modulation, and/or by a depletion of the precancerous or cancerous cells. Target gene modulation can be of a negative or positive nature, depending on whether the target resembles a gene (for example, tumorigenic) or a tumor suppressor gene (for example, tumor suppressive). That is, inhibition, i.e., a negative modulation, of an oncogene-like target gene or stimulation, i.e., a positive modulation, of a tumor suppressor-like target gene will control or ameliorate the tumor or cancer in which the target gene is involved. More precisely, “negative modulation” refers to a reduction in the level and/or activity of target gene or its product, such as Galanin protein, GALR2 protein, GALR3 protein, or GALR2-GALR3 heterocomplex protein, relative to the level and/or activity of the target gene product in the absence of the modulatory treatment. “Positive modulation” refers to an increase in the level and/or activity of target gene product, such as Galanin protein, GALR2 protein, GALR3 protein, or GALR2-GALR3 heterocomplex protein, relative to the level and/or activity of target gene or its product in the absence of modulatory treatment. Particularly because Galanin, GALR2, or GALR3 shares many features with well known oncogenes as discussed supra, inhibition of the Galanin, GALR2, or GALR3 gene, its protein, or its activities will control or ameliorate cancerous conditions, for example, lung cancer and/or brain cancer and/or breast cancer and/or colon cancer and/or prostate cancer and/or ovarian cancer.

The techniques to inhibit or suppress a target gene such as Galanin, GALR2, or GALR3 that is involved in cancers, i.e., the negative modulatory techniques are provided in the present invention. For example, compounds that exhibit negative modulatory activity on Galanin, GALR2, GALR3, or GALR2-GALR3 heterocomplex can be used in accordance with the invention to prevent and/or ameliorate symptoms of tumors and cancers (for example, lung cancer, brain cancer, breast cancer, colon cancer, prostate cancer, or ovarian cancer). Such molecules can include, but are not limited to, peptides, phosphopeptides, small molecules (molecular weight below about 500), large molecules (molecular weight above about 500), or antibodies (including, for example, polyclonal, monoclonal, humanized, anti-idiotypic, chimeric or single chain antibodies, and Fab, F(ab′)2 and Fab expression library fragments, and epitope-binding fragments thereof), and nucleic acid molecules that interfere with replication, transcription, or translation of the Galanin, GALR2, or GALR3 gene (for example, antisense nucleic acid molecules, siRNAs, triple helix forming molecules, and ribozymes, which can be used alone or in any combination).

Antisense, siRNAs and ribozyme molecules that inhibit expression of a target gene such as Galanin, GALR2, or GALR3 may reduce the level of the functional activities of the target gene and its product, for example, reduce the catalytic potency of Galanin, GALR2, or GALR3, respectively. Triple helix forming molecules, also related, can be used in reducing the level of target gene activity. These molecules can be designed to reduce or inhibit′ either wild type, or if appropriate, mutant target gene activity.

For example, anti-sense RNA and DNA molecules act to directly block the translation of mRNA by hybridizing to targeted mRNA and preventing protein translation. With respect to antisense DNA, oligodeoxyribonucleotides derived from the translation initiation site, for example, between the −10 and +10 regions of the target gene nucleotide sequence of interest, are preferred.

Ribozymes are enzymatic RNA molecules capable of catalyzing the specific cleavage of RNA. A review is provided in Rossi, Current Biology, 4:469-471 (1994). The mechanism of ribozyme action involves sequence-specific hybridization of the ribozyme molecule to complementary target RNA, followed by an endonucleolytic cleavage. A composition of ribozyme molecules must include one or more sequences complementary to the target gene mRNA, and must include a well-known catalytic sequence responsible for mRNA cleavage (U.S. Pat. No. 5,093,246). Engineered hammerhead motif ribozyme molecules that may specifically and efficiently catalyze internal cleavage of RNA sequences encoding target protein such as Galanin, GALR2, or GALR3 may be used according to this invention in cancer intervention.

Specific ribozyme cleavage sites within any potential RNA target are initially identified by scanning the molecule of interest, for example, Galanin, GALR2, or GALR3 RNA, for ribozyme cleavage sites which include the following sequences, GUA, GUU and GUC. Once identified, short RNA sequences of between 15 and 20 ribonucleotides corresponding to the region of the target gene such as Galanin, GALR2, or GALR3 containing the cleavage site can be evaluated for predicted structural features, such as secondary structure, that can render an oligonucleotide sequence unsuitable. The suitability of candidate sequences can also be evaluated by testing their accessibility to hybridization with complementary oligonucleotides, using ribonuclease protection assays.

The Galanin, GALR2, and GALR3 gene sequences also can be employed in an RNA interference context. The phenomenon of RNA interference is described and discussed in Bass, Nature, 411: 428-29 (2001); Elbashir et al., Nature, 411: 494-98 (2001); and Fire et al., Nature, 391: 806-11 (1998), where methods of making interfering RNA also are discussed. The siRNAs based upon the sequence disclosed herein (for example, GenBank Accession Number NM_{—}003857 and NM_{—}003614 for Galanin, GALR2, and GALR3 mRNA sequences, respectively) is typically less than 100 base pairs (“bps”) in length and constituency and preferably is about 30 bps or shorter, and can be made by approaches known in the art, including the use of complementary DNA strands or synthetic approaches. The RNAs that are capable of causing interference can be referred to as small interfering RNAs (“siRNA”), and can cause post-transcriptional silencing of specific genes in cells, such as mammalian cells (including human cells) and in the body, such as mammalian bodies (including humans). Exemplary siRNAs according to the invention could have up to 29 bps, 25 bps, 22 bps, 21 bps, 20 bps, 15 bps, 10 bps, 5 bps or any number thereabout or therebetween.

Nucleic acid molecules that can associate together in a triple-stranded conformation (triple helix) and that thereby can be used to inhibit transcription of a target gene, should be single helices composed of deoxynucleotides. The base composition of these oligonucleotides must be designed to promote triple helix formation via Hoogsteen base pairing rules, which generally require sizeable stretches of either purines or pyrimidines on one strand of a duplex. Nucleotide sequences can be pyrimidine-based, which will result in TAT and CGC triplets across the three associated strands of the resulting triple helix. The pyrimidine-rich molecules provide bases complementary to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines on one strand of a duplex.

In instances wherein the antisense, ribozyme, siRNA, and triple helix forming molecules described herein are used to reduce or inhibit mutant gene expression, it is possible that they can also effectively reduce or inhibit the transcription (for example, using a triple helix) and/or translation (for example, using antisense, ribozyme molecules) of mRNA produced by the normal target gene allele. These situations are pertinent to tumor suppressor genes whose normal levels in the cell or tissue need to be maintained while a mutant is being inhibited. To do this, nucleic acid molecules which are resistant to inhibition by any antisense, ribozyme or triple helix forming molecules used, and which encode and express target gene polypeptides that exhibit normal target gene activity, can be introduced into cells via gene therapy methods. Alternatively, when the target gene encodes an extracellular protein, it may be preferable to co-administer normal target gene protein into the cell or tissue to maintain the requisite level of cellular or tissue target gene activity. By contrast, in the case of oncogene-like target genes such as Galanin, GALR2, or GALR3, it is the respective normal wild type Galanin, GALR2, or GALR3 gene and its protein that need to be suppressed. Thus, any mutant or variants that are defective in Galanin, GALR2, or GALR3 function or that interferes or completely abolishes its normal function would be desirable for cancer treatment. Therefore, the same methodologies described above to safeguard normal gene alleles may be used in the present invention to safeguard the mutants of the target gene in the application of antisense, ribozyme, and triple helix treatment.

Anti-sense RNA and DNA, ribozyme, and triple helix forming molecules of the invention can be prepared by standard methods known in the art for the synthesis of DNA and RNA molecules. These include techniques for chemically synthesizing oligodeoxyribonucleotides and oligoribonucleotides well known in the art such as, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which also include suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules can be introduced as a means for increasing intracellular stability and half-life. Possible modifications include, but are not limited to, the addition of flanking sequences of ribo- or deoxy- nucleotides to the 5′ and/or 3′ ends of the molecule, or the use of phosphorothioate or 2′ O-methyl rather than phosphodiesterase linkages within the oligodeoxyribonucleotide backbone.

In this aspect, the present invention also provides negative modulatory techniques using antibodies. Antibodies can be generated which are both specific for a target gene product and which reduce target gene product activity; they can be administered when negative modulatory techniques are appropriate for the treatment of tumors and cancers, for example, in the case of Galanin, GALR2, GALR3, or GALR2-GALR3 heterocomplex antibodies for lung cancer, brain cancer, breast cancer, colon cancer, prostate cancer, or ovarian cancer treatment.

In instances where the target gene protein to which the antibody is directed is intracellular, and whole antibodies are used, internalizing antibodies are preferred. However, lipofectin or liposomes can be used to deliver the antibody, or a fragment of the Fab region which binds to the target gene epitope, into cells. Where fragments of an antibody are used, the smallest inhibitory fragment which specifically binds to the binding domain of the protein is preferred. For example, peptides having an amino acid sequence corresponding to the domain of the variable region of the antibody that specifically binds to the target gene protein can be used. Such peptides can be synthesized chemically or produced by recombinant DNA technology using methods well known in the art (for example, see Creighton, 1983, supra; and Sambrook et al., 1989, supra). Alternatively, single chain neutralizing antibodies that bind to intracellular target gene product epitopes also can be administered. Such single chain antibodies can be administered, for example, by expressing nucleotide sequences encoding single-chain antibodies within the target cell population by using, for example, techniques such as those described in Marasco et al., Proc. Natl. Acad. Sci. U.S.A., 90:7889-7893 (1993). When the target gene protein is extracellular, or is a transmembrane protein, any of the administration techniques known in the art which are appropriate for peptide administration can be used to effectively administer inhibitory target gene antibodies to their site of action. The methods of administration and pharmaceutical preparations are discussed below.

G. Cancer Vaccines Using Galanin, GALR2, or GALR3:

One aspect of the invention relates to methods for inducing an immunological response in a mammal which comprises inoculating the mammal with Galanin, GALR2, or GALR3 polypeptide, or a fragment thereof, adequate to produce antibody and/or T cell immune response to protect the mammal from cancers, including ovarian cancer.

In another aspect, the present invention relates to peptides derived from the Galanin, GALR2, or GALR3 amino acid sequence (for example, SEQ ID NO:8, SEQ ID NO:4, or SEQ ID NO:2), where those skilled in the art would be aware that the peptides of the present invention, or analogs thereof, can be synthesized by automated instruments sold by a variety of manufacturers, can be commercially custom ordered and prepared, or can be expressed from suitable expression vectors as described above. The term amino acid analogs has been previously described in the specification and for purposes of describing peptides of the present invention, analogs can further include branched or non-linear peptides.

The present invention therefore provides pharmaceutical compositions comprising Galanin, GALR2, GALR3, or GALR2-GALR3 heterocomplex, protein or peptides derived therefrom for use in vaccines and in immunotherapy methods. When used as vaccines to protect mammals against cancer, the pharmaceutical composition can comprise as an immunogen cell lysate from cells transfected with a recombinant expression vector or a culture supernatant containing the expressed protein. Alternatively, the immunogen is a partially or substantially purified recombinant protein or a synthetic peptide.

Vaccination can be conducted by conventional methods. For example, the immunogen can be used in a suitable diluent such as saline or water, or complete or incomplete adjuvants. Further, the immunogen may or may not be bound to a carrier to make the protein immunogenic. Examples of such carrier molecules include but are not limited to bovine serum albumin (BSA), keyhole limpet hemocyanin (KLH), tetanus toxoid, and the like. The immunogen can be administered by any route appropriate for antibody production such as intravenous, intraperitoneal, intramuscular, subcutaneous, and the like. The to immunogen may be administered once or at periodic intervals until a significant titer of anti-Galanin, GALR2, GALR3, or GALR2-GALR3 heterocomplex antibody is produced. The antibody may be detected in the serum using an immunoassay.

In yet another aspect, the present invention provides pharmaceutical compositions comprising nucleic acid sequence capable of directing host organism synthesis of an Galanin, GALR2, or GALR3 protein or of a peptide derived from the Galanin, GALR2, or GALR3 protein sequence. Such nucleic acid sequence may be inserted into a suitable expression vector by methods known to those skilled in the art. Expression vectors suitable for producing high efficiency gene transfer in vivo include, but are not limited to, retroviral, adenoviral and vaccinia viral vectors. Operational elements of such expression vectors are disclosed previously in the present specification and are known to one skilled in the art. Such expression vectors can be administered, for example, intravenously, intramuscularly, subcutaneously, intraperitoneally or orally.

Whether the immunogen is an Galanin, GALR2, GALR3, or GALR2-GALR3 heterocomplex, protein, a peptide derived therefrom or a nucleic acid sequence capable of directing host organism synthesis of Galanin, GALR2, or GALR3 protein or peptides derived therefrom, the immunogen may be administered for either a prophylactic or therapeutic purpose. Such prophylactic use may be appropriate for, for example, individuals with a genetic predisposition to a particular cancer. When provided prophylactically, the immunogen is provided in advance of the cancer or any symptom due to the cancer. The prophylactic administration of the immunogen serves to prevent or attenuate any subsequent onset of cancer. When provided therapeutically, the immunogen is provided at, or shortly after, the onset of cancer or any symptom associated with the cancer.

The present invention further relates to a vaccine for immunizing a mammal, for example, humans, against cancer comprising Galanin, GALR2, GALR3, or GALR2-GALR3 heterocomplex, protein or an expression vector capable of directing host organism synthesis of Galanin, GALR2, or GALR3 protein in a pharmaceutically acceptable carrier.

In addition to use as vaccines and in immunotherapy, the above compositions can be used to prepare antibodies to Galanin, GALR2, GALR3, or GALR2-GALR3 heterocomplex. To prepare antibodies, a host animal is immunized using the Galanin, GALR2, GALR3, or GALR2-GALR3 heterocomplex, protein or peptides derived therefrom or aforementioned expression vectors capable of expressing Galanin, GALR2, or GALR3 protein or peptides derived therefrom. The host serum or plasma is collected following an appropriate time interval to provide a composition comprising antibodies reactive with the virus particle. The gamma globulin fraction or the IgG antibodies can be obtained, for example, by use of saturated ammonium sulfate or DEAE Sephadex, or other techniques known to those skilled in the art. The antibodies are substantially free of many of the adverse side effects which may be associated with other anti-viral agents such as drugs.

The antibody compositions can be made even more compatible with the host system by minimizing potential adverse immune system responses. This is accomplished by removing all or a portion of the Fc portion of a foreign species antibody or using an antibody of the same species as the host animal, for example, the use of antibodies from human/human hybridomas. Humanized antibodies (i.e., nonimmunogenic in a human) may be produced, for example, by replacing an immunogenic portion of a non-human antibody with a corresponding, but nonimmunogenic portion (i.e., chimeric antibodies). Such chimeric antibodies may contain the reactive or antigen binding portion of an antibody from one species and the Fc portion of an antibody (nonimmunogenic) from a different species. Examples of chimeric antibodies, include but are not limited to, non-human mammal-human chimeras, rodent-human chimeras, murine-human and rat-human chimeras (Cabilly et al, Proc. Natl. Acad. Sci. USA, 84:3439, 1987; Nishimura et al., Cancer Res., 47:999, 1987; Wood et al., Nature, 314:446, 1985; Shaw et al., J. Natl. Cancer Inst., 80:15553,1988). General reviews of “humanized” chimeric antibodies are provided by Morrison S., Science, 229:1202, 1985 and by Oi et al., BioTechniques, 4:214, 1986.

Alternatively, anti-Galanin, -GALR2, -GALR3, or -GALR2-GALR3 heterocomplex antibodies can be induced by administering anti-idiotype antibodies as immunogen. Conveniently, a purified anti-Galanin, -GALR2, -GALR3, or -GALR2-GALR3 heterocomplex antibody preparation prepared as described above is used to induce anti-idiotype antibody in a host animal. The composition is administered to the host animal in a suitable diluent. Following administration, usually repeated administration, the host produces anti-idiotype antibody. To eliminate an immunogenic response to the Fc region, antibodies produced by the same species as the host animal can be used or the Fc region of the administered antibodies can be removed. Following induction of anti-idiotype antibody in the host animal, serum or plasma is removed to provide an antibody composition. The composition can be purified as described above for anti-Galanin, -GALR2, -GALR3, or -GALR2-GALR3 heterocomplex antibodies, or by affinity chromatography using anti-Galanin, -GALR2, -GALR3, or -GALR2-GALR3 heterocomplex antibodies bound to the affinity matrix. The anti-idiotype antibodies produced are similar in conformation to the authentic Galanin, GALR2, GALR3, or GALR2-GALR3 heterocomplex antigen and may be used to prepare vaccine rather than using a Galanin, a GALR2, a GALR3, or a GALR2-GALR3 heterocomplex.

When used as a means of inducing anti-Galanin, -GALR2, -GALR3, or -GALR2-GALR3 heterocomplex antibodies in an animal, the manner of injecting the antibody is the same as for vaccination purposes, namely intramuscularly, intraperitoneally, subcutaneously or the like in an effective concentration in a physiologically suitable diluent with or without adjuvant. One or more booster injections may be desirable.

For both in vivo use of antibodies to Galanin, GALR2, GALR3, or GALR2-GALR3 heterocomplex and anti-idiotype antibodies and for diagnostic use, it may be preferable to use monoclonal antibodies. Monoclonal anti-Galanin, -GALR2, -GALR3, or -GALR2-GALR3 heterocomplex antibodies or anti-idiotype antibodies can be produced by methods known to those skilled in the art. (Goding, J. W. 1983. Monoclonal Antibodies: Principles and Practice, Pladermic Press, Inc., NY, N.Y., pp. 56-97). To produce a human-human hybridoma, a human lymphocyte donor is selected. A donor known to have the Galanin, GALR2, GALR3, or GALR2-GALR3 heterocomplex antigen may serve as a suitable lymphocyte donor. Lymphocytes can be isolated from a peripheral blood sample or spleen cells may be used if the donor is subject to splenectomy. Epstein-Barr virus (EBV) can be used to immortalize human lymphocytes or a human fusion partner can be used to produce human-human hybridomas. Primary in vitro immunization with peptides can also be used in the generation of human monoclonal antibodies.

H. Pharmaceutical Applications of Compounds:

The identified compounds that inhibit the expression, synthesis, and/or activity of the target gene such as Galanin, GALR2, or GALR3 can be administered to a patient at therapeutically effective doses to prevent, treat, or control a tumor or cancer. A therapeutically effective dose refers to an amount of the compound that is sufficient to result in a measurable reduction or elimination of cancer or its symptoms.

Toxicity and therapeutic efficacy of such compounds can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from the cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

Pharmaceutical compositions for use in the present invention can be formulated by standard techniques using one or more physiologically acceptable carriers or excipients. The compounds and their physiologically acceptable salts and solvates can be formulated and administered, for example, orally, intraorally, rectally, parenterally, epicutaneously, topically, transdermally, subcutaneously, intramuscularly, intranasally, sublingually, intradurally, intraocularly, intrarespiratorally, intravenously, intraperitoneally, intrathecal, mucosally, by oral inhalation, nasal inhalation, or rectal administration, for example.

For oral administration, the pharmaceutical compositions can take the form of tablets or capsules prepared by conventional means with pharmaceutically acceptable excipients, for example, binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. The tablets can be coated by methods well known in the art. Liquid preparations for oral administration can take the form of solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. Preparations for oral administration can be suitably formulated to give controlled release of the active compound.

For administration by inhalation, the compounds are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use. The compounds can also be formulated in rectal compositions, for example, suppositories or retention enemas, for example, containing conventional suppository bases, for example, cocoa butter or other glycerides.

Furthermore, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which to can contain one or more unit dosage forms containing the active ingredient. The pack can for example comprise metal or plastic foil, for example, a blister pack. The pack or dispenser device can be accompanied by instructions for administration.

I. Administration of siRNA/shRNA:

The invention includes methods of administering siRNA, to a patient in need thereof, wherein the siRNA/shRNA molecule is delivered in the form of a naked oligonucleotide or via an expression vector as described herein.

The present invention provide methods of blocking the in vivo expression of Galanin, GALR2 or GALR3 gene by administering a naked DNA or a vector containing siRNA/shRNA as set forth herein (see, for example, Example IX), which interacts with the target gene and cause post-transcriptional silencing of specific genes in cells, for example, mammalian cells (including human cells) and in the body, for example, mammalian bodies (including humans).

The invention also provide methods for the treatment of cells ex vivo by administering a naked DNA or a vector according to the invention.

In its in vivo or ex vivo therapeutic applications, it is appropriate to administer siRNA/shRNA using a viral or retroviral vector, which enters the cell by transfection or infection. In particular, as a therapeutic product according to the invention, a vector can be a defective viral vector, such as an adenovirus, or a defective retroviral vector, such as a murine retrovirus.

The vector used to convey the gene construct according to the invention to its target can be a retroviral vector, which will transport the recombinant construct by a borrower capsid, and insert the genetic material into the DNA of the host cell.

Techniques that use vectors, in particular viral vectors (retroviruses, adenoviruses, adeno-associated viruses), to transport genetic material to target cells can be used to introduce genetic modifications into various somatic tissues, for example, breast, colon, lung, brain, prostate or ovarian cells.

The use of retroviral vectors to transport genetic material necessitates, on the one hand, carrying out the genetic construction of the recombinant retrovirus, and on the other hand having a cell system available which provides for the function of encapsidation of the genetic material to be transported:

i. In a first stage, genetic engineering techniques enable the genome of a murine retrovirus, such as Moloney virus (murine retrovirus belonging to the murine leukemia virus group (Reddy et al., Science, 214:445-450 (1981)). The retroviral genome is cloned into a plasmid vector, from which all the viral sequences coding for the structural proteins (genes: Gag, Env) as well as the sequence coding for the enzymatic activities (gene: Pol) are then deleted. As a result, only the necessary sequences “in cis” for replication, transcription and integration are retained (sequences corresponding to the two LTR regions, encapsidation signal and primer binding signal). The deleted genetic sequences may be replaced by non-viral genes such as the gene for resistance to neomycin (selection antibiotic for eukaryotic cells) and by the gene to be transported by the retroviral vector, for example, Galanin and/or GALR2 and/or GALR3 siRNA as set forth herein.

ii. In a second stage, the plasmid construct thereby obtained is introduced by transfection into the encapsidation cells. These cells constitutively express the Gag, Pol and Env viral proteins, but the RNA coding for these proteins lacks the signals needed for its encapsidation. As a result, the RNA cannot be encapsidated to enable viral particles to be formed. Only the recombinant RNA emanating from the transfected retroviral construction is equipped with the encapsidation signal and is encapsidated. The retroviral particles produced by this system contain all the elements needed for the infection of the target cells (such as CD34+ cells) and for the permanent integration of the gene of interest into these cells, for example, Galanin and/or GALR2 and/or GALR3 siRNA as set forth herein. The absence of the Gag, Pol and Env genes prevents the system from continuing to propagate.

DNA viruses such as adenoviruses also can be suited to this approach although, in this case, maintenance of the DNA in the episomal state in the form of an autonomous replicon is the most likely situation.

Adenoviruses possess some advantageous properties. In particular, they have a fairly broad host range, are capable of infecting quiescent cells and do not integrate into the genome of the infected cell. For these reasons, adenoviruses have already been used for the transfer of genes in vivo. To this end, various vectors derived from adenoviruses have been prepared, incorporating different genes (beta-gal, OTC, alpha-1At, cytokines, etc.). To limit the risks of multiplication and the formation of infectious particles in vivo, the adenoviruses used are generally modified so as to render them incapable of replication in the infected cell. Thus, the adenoviruses used generally have the E1 (E1a and/or E1b) and possibly E3 regions deleted.

The defective recombinant adenoviruses according to the invention may be prepared by any technique known to persons skilled in the art (Levrero et al., Gene, 101:195 (1991), EP 185 573; Graham, EMBO J. 3:2917 (1984)). In particular, they may be prepared by homologous recombination between an adenovirus and a plasmid in a suitable cell line.

According to the present invention, an exogenous DNA sequence, for example, Galanin and/or GALR2 and/or GALR3 siRNA as set forth herein, is inserted into the genome of the defective recombinant adenovirus.

Pharmaceutical compositions comprising one or more viral vectors, such as defective recombinants as described above, may be formulated for the purpose of topical, oral, parenteral, intranasal, intravenous, intramuscular, subcutaneous, intraocular, and the like, administration. Preferably, these compositions contain vehicles which are pharmaceutically acceptable for an administrable formulation. These can be, in particular, isotonic, sterile saline solutions (of monosodium or disodium phosphate, sodium, potassium, calcium or magnesium chloride, and the like, or mixtures of such salts), or dry, in particular lyophilized, compositions which, on addition, as appropriate, of sterilized water or of physiological saline, enable particular injectable solutions to be made up.

The doses of defective recombinant virus used for the injection may be adapted in accordance with various parameters, and in particular in accordance with the mode of administration used, the pathology in question, the gene to be expressed or the desired duration of treatment. Generally speaking, the recombinant adenoviruses according to the invention may be formulated and administered in the form of doses of between 10⁴ and 10¹⁴ pfu/ml, and preferably 10⁶ to 10¹⁰ pfu/ml. The term pfu (“plaque forming unit”) corresponds to the infectious power of a solution of virus, and is determined by infection of a suitable cell culture and measurement, generally after 48 hours, of the number of plaques of infected cells. The techniques of determination of the pfu titer of a viral solution are well documented in the literature.

The use of genetically modified viruses as a shuttle system for transporting the modified genetic material not only permits the genetic material to enter the recipient cell by the expedient of using a borrower viral capsid, but also allows a large number of cells to be treated simultaneously and over a short period of time, which permits therapeutic treatment applied to the whole body.

The invention is further described by the following examples, which do not limit the invention in any manner.

### EXAMPLES

### Example I

### Amplification of GALR3 Gene in Lung Tumors and Lung Cancer Cell Lines

Referring to Table 4, the DNA copy numbers were determined in the primary lung cancer samples using a Taqman 7700 and 7900 Sequence Detectors. The genomic DNAs were isolated from lung tumors and lung cancer cell lines. A total of 76 primary lung and 16 lung cancer cell lines were subjected, along with the same GALR3 and GALR2 Taqman probe representing the target, and a reference probe representing a normal non-amplified, single copy region in the genome, to analysis by TaqMan 7700 Sequence Detector (Applied Biosystems) following the manufacturer's protocol. Out of 76 primary lung tumors tested, 21 and 18 were found to be over 5 fold or higher for GALR3 and GALR2, respectively. Using 2.5 fold as a cutoff, 34 of the 76 samples were found to have increased copy number for both GALR2 and GALR3. Of the 16 lung cancer cell lines tested, colo699 was the only one found amplified for GALR3 gene at more than 2.5 fold. Only samples with the GALR3 gene copy number greater than or equal to 2.5 fold are deemed to have been amplified, because of the instrumental detection limit. However, an increase in GALR3 gene copy number less than 2.5 fold can still be considered as an amplification of the gene, if detected.

TABLE 4
GALR3 is frequently amplified in primary human lung tumors.

| Lung | | |
|---|---|---|
| Tumors | GALR3 DNA Copy Number | GALR2 DNA Copy Number |
| LU84 | 58 | 3.4 |
| LU73 | 32 | 16 |
| LU83 | 26 | 12 |
| LU82 | 11 | 7.8 |
| LU74 | 10 | 8.1 |
| LU50 | 9.7 | 7.6 |
| LU62 | 9.7 | 4.9 |
| LU64 | 8.8 | 1.7 |
| LU63 | 8.5 | 1.9 |
| LU71 | 7.4 | 6.4 |
| LU79 | 7 | 4.2 |
| LU85 | 6.8 | 3.7 |
| LU81 | 6.5 | 3.3 |
| LU66 | 6.1 | 2.3 |
| LU45 | 6 | 4.2 |
| LU75 | 5.8 | 6.9 |
| LU51 | 5.4 | 6.9 |
| LU52 | 5.4 | 4.7 |
| LU55 | 5.1 | 12 |
| LU15 | 5.1 | 6 |
| LU78 | 5.1 | 2.4 |
| LU80 | 4.9 | 3.6 |
| LU58 | 4.6 | 8.3 |
| LU59 | 4.6 | 7.8 |
| LU49 | 4.4 | 3.6 |
| LU65 | 4.4 | 1.4 |
| LU6 | 4.3 | 7.7 |
| LU21 | 4.1 | 4.4 |
| LU76 | 4.1 | 3.5 |
| LU54 | 3.8 | 4.7 |
| LU1 | 3.6 | 4.6 |
| LU30 | 3.5 | 3.7 |
| LU48 | 3.5 | 3.5 |
| LU72 | 3.3 | 2.5 |
| LU69 | 3.2 | 0.62 |
| LU61 | 3.1 | 4.6 |
| LU13 | 3.1 | 1.5 |
| LU24 | 2.9 | 6.5 |
| LU5 | 2.9 | 2.8 |
| LU67 | 2.8 | 0.95 |
| LU60 | 2.7 | 3.8 |
| LU70 | 2.7 | 2.2 |
| LU43 | 2.6 | 3.2 |
| LU23 | 2.4 | 3.5 |
| LU9 | 2.3 | 6.1 |
| LU22 | 2.2 | 22.4 |
| LU29 | 2.2 | 3.4 |
| LU12 | 2.2 | 3.3 |
| LU47 | 2.1 | 4 |
| LU4 | 2.1 | 3.7 |
| LU7 | 2 | 5.9 |
| LU8 | 1.9 | 3.8 |
| LU17 | 1.8 | 4.6 |
| LU42 | 1.7 | 4.2 |
| LU25 | 1.7 | 3.7 |
| LU11 | 1.7 | 3.6 |
| LU20 | 1.7 | 3.1 |
| LU68 | 1.7 | 0.82 |
| LU39 | 1.6 | 5.2 |
| LU37 | 1.5 | 3.5 |
| LU77 | 1.5 | 3.2 |
| LU2 | 1.3 | 2.2 |
| LU35 | 1.2 | 2.8 |
| LU16 | 1.2 | 2.1 |
| LU19 | 1.1 | 4.7 |
| LU28 | 1.1 | 3.5 |
| LU44 | 1.1 | 3.5 |
| LU27 | 1.1 | 2.5 |
| LU14 | 1.1 | 2.1 |
| LU34 | 1.1 | 1.4 |
| LU18 | 1 | 1.9 |
| LU3 | 0.95 | 1.6 |
| LU26 | 0.74 | 2.4 |
| LU10 | 0.69 | 2.5 |
| LU36 | 0.37 | 1.2 |

### Example II

### Overexpression of the GALR3 and GALR2 Genes in Lung Tumors and Cancer Cell Lines

Reverse transcriptase (RT)-directed quantitative PCR was performed using the TaqMan 7700 Sequence Detector (Applied Biosystems) to determine the GALR3 and GALR2 mRNA level in each sample. Human beta-actin mRNA was used as control.

Total RNA was isolated from human lung tumors and lung tumor cell lines using Trizol Reagent (Invitrogen) and treated with DNAase (Ambion) to eliminate genomic DNA. The reverse transcriptase reaction (at 48° C. for 30 min) was coupled with quantitative PCR measurement of cDNA copy number in a one-tube format according to the manufacturer (Perkin Elmer/Applied Biosystems). GALR3 and GALR2 expression level in the samples was normalized using human β-actin and overexpression fold was calculated by comparing GALR3 and GALR2 expression in tumor v. normal samples. The nucleotide sequences of GALR3 and GALR2 are used to design suitable Taqman probes based on the sequences listed under the GenBank Accession Number NM_{—}003614 (GALR3) and NM_{—}003857 (GALR2). The measurements of the mRNA level of each primary tumor and tumor cell line sample were normalized to the mRNA levels in normal lung tissue and normal human bronchial epithelial (NHBE) cell line, respectively. Relative numerical values of the mRNA levels are shown in Tables 5 and 6. Of the 10 lung tumors studied shown in Table 5, all overexpress GALR3 and GALR2 at varying degree, including the sample LU44 which lacks GALR3 gene amplification. Of the 11 lung cancer cell lines studies (Table 6), all 11 overexpress GALR3 5 and 7 of 11 overexpress GALR2 at 5 fold or more. The observation of GALR message overexpression without concomitant gene amplification is reminiscent of a hallmark of oncogenes such as c-myc (Yoshimoto et al. Jpn. J. Cancer Res. 77, 540-545 (1986)) and AIB1 (Anzick et al. Science 277, 965-968 (1997)).

TABLE 5
Amplification status and overexpression of GALR3 and GALR2
in lung tumors and relative numerical values of the mRNA levels.

| | GALR3 | | GALR2 | |
|---|---|---|---|---|
| | DNA | Relative | DNA | Relative |
| Lung | Copy | GALR3 mRNA | Copy | GALR2 mRNA |
| Tumors | Number | Level | Number | Level |
| LU1 | 3.6 | 5.9 | 4.6 | 71 |
| LU5 | 2.9 | 107 | 2.8 | 18 |
| LU30 | 3.5 | 182 | 3.7 | 22 |
| LU44 | 1.1 | 26 | 3.8 | 4.4 |
| LU45 | 5.3 | 49 | 4.7 | 5.1 |
| LU49 | 4.4 | 164 | 3.7 | 15 |
| LU52 | 5.4 | 5.8 | 4.7 | 20 |
| LU54 | 3.8 | 27 | 4.7 | 3.2 |
| LU82 | 3.5 | 29 | 3.3 | 10 |
| LU85 | 3.3 | 127 | 3.3 | 4.7 |

TABLE 6
Amplification status and overexpression of GALR3 and GALR2 in
lung cancer cell lines and relative numerical values of the mRNA levels.

| | GALR3 | | GALR2 | |
|---|---|---|---|---|
| | DNA | Relative | DNA | Relative |
| Lung Cancer | Copy | GALR3 mRNA | Copy | GALR2 mRNA |
| Cell Lines | Number | Level | Number | Level |
| 9812 | 0.55 | 69 | 0.98 | 25 |
| NCI-H128 | 0.31 | 21 | 0.68 | 8.5 |
| LCLC-103H | 0.79 | 37 | 1.1 | 0.66 |
| LOU-NH91 | 0.24 | 18 | 1.2 | 0.19 |
| colo699 | 4.5 | 666 | 2.2 | 38 |
| DMS114 | 0.37 | 28 | 1.5 | 2.1 |
| H510A | 0.11 | 161 | 0.98 | 10 |
| BEN | 0.32 | 38 | 1.3 | 5.7 |
| NCI-H727 | 1.3 | 14 | 1.4 | 2.3 |
| SKLC-13 | 2.1 | 65 | 2.1 | 41 |
| SKLU-1 | 1.5 | 6.2 | 1.9 | 14 |
| NHBE | 1.2 | 1 | 1.1 | 1 |

### Example III

### Physical Map of the Amplicon Containing the Galanin, GALR2 and GALR3 Genes

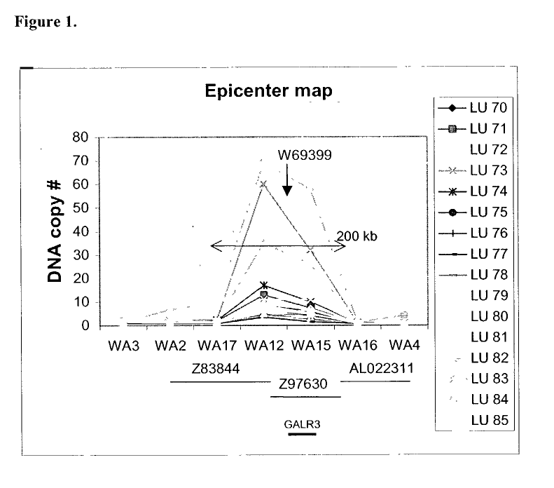

Cancer cell lines or primary tumors were examined for DNA copy number of genes and markers near GALR3, GALR2, and Galanin to map the boundaries of the amplified regions. It is demonstrated that GALR3, GALR2, and Galanin are located at the epicenter of the amplification regions (FIGS. 1, 2 and 3).

DNA was purified from tumor cell lines or primary tumors. The DNA copy number of each marker in each sample was directly measured using PCR and a fluorescence-labeled probe. The number of PCR cycles needed to cross a preset threshold, also known as Ct value, in the sample tumor DNA preparations and a series of normal human DNA preparations at various concentrations was measured for both the target probe and a known single-copy DNA probe using Applied Biosystems 7700 TaqMan machine. The relative abundance of target sequence to the single-copy probe in each sample was then calculated by statistical analyses of the Ct values of the unknown samples and the standard curve generated from the normal human DNA preparations at various concentrations.

To determine the DNA copy number for each of the genes, corresponding probes to each marker were designed using PrimerExpress 1.0 (Applied Biosystems) and synthesized by Operon Technologies. Subsequently, the target probe (representing the marker), a reference probe (representing a normal non-amplified, single copy region in the genome), and tumor genomic DNA (10 ng) were subjected to analysis by the Applied Biosystems 7700 TaqMan Sequence Detector following the manufacturer's protocol. The number of DNA copies for each sample was plotted against the corresponding marker in FIGS. 1, 2 and 3. Figures show the epicenter mapping of 22q13, 17q25, and 11q13 amplicons, which include GALR3, GALR2, and Galanin loci. The number of DNA copies for each sample is plotted on the Y-axis, and the X-axis corresponds to nucleotide position based on Human Genome Project working draft sequence (http://genome.ucsc.eclu/goldenPath/aug2001Tracks.html).

Referring to FIG. 1 (GALR3), based on the Human Genome Browser (http://genome.ucsc.edu/goldenPath/aug2001Tracks.html), three human genomic DNA clones are shown (Z83844, Z97630, and AL022311), but not to the scale of actual clone sizes. The epicenter which is about 200 kb in size is completely contained with these 3 genomic sequences. The 7 markers are placed at equal interval (not to the scale of actual distance) on X-axis for viewing purpose. WA3, bases 643-714 of genomic DNA clone AL022315; WA2, bases 488-562 of genomic DNA clone Z83844; WA17, bases 65146-65214 of genomic DNA clone Z83844; WA12, bases 131949-132033 of Z83844; WA15, bases 31437-31502 of Z97630; WA16, bases 16961-17057 of AL022311; WA4, bases 69682-69745 of AL022311.

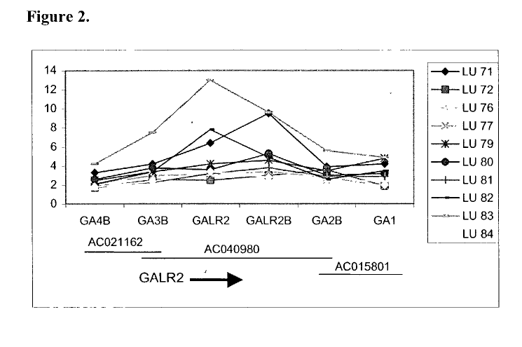

Referring to FIG. 2 (GALR2), based on the Human Genome Browser (http://genome.ucsc.edu/goldenPath/aug2001Tracks.html), three human genomic DNA clones are shown (AC021162, AC040980, and AC015801) not to the scale of actual clone sizes. GALR2 gene is indicated by an arrow. GA4B, bases 10540-10640 of human genomic DNA clone Accession Number AC021162; GA3B, bases 196815-196875 of AC021162; GALR2, bases 131772-131823 of AC040980; GALR2B, 130310-130362 of AC040980; GA2B, bases 4642-4706 of AC040980; GA1, bases 171-245 of AC015801.

Referring to FIG. 3, based on the Human Genome Project working draft sequence (http://genome.ucsc.edu/goldenPath/aug2001Tracks.html), depicting an epicenter at 11q13 containing the Galanin gene. The number of DNA copies for each sample is plotted on the Y-axis, and the X-axis corresponds to nucleotide position. The epicenter of approximately 150 -kb in size is contained within three overlapping human genomic DNA clones as shown in dotted lines (from left to right: AP001075, AP001788, AP003732). The 14 markers include (left to right): GR4, 100 kb; GR21, 200 kb; GR3, 295 kb; GRI, 365 kb; GR22, 395 kb; GR7, 425 kb; GR8, 428 kb; GR 9, 432 kb; GAA, 435 kb;.GR5, 490 kb; GR24, 560 kb; GR24A, 570 kb; GR6, 740 kb; GR25, 750 kb.

### Example IV

### Amplification and Overexpression of the Galanin Gene in Lung Tumors

Based on DNA microarray-based CGH to survey the genome for gene amplification, it was discovered that the Galanin gene is frequently amplified in tumor tissues.

The genomic DNAs were isolated from tumor samples. They were subjected, along with the same Galanin TaqMan probe representing the target, and a reference probe representing a normal non-amplified, single copy region in the genome, to analysis by TaqMan 7700 Sequence Detector (Applied Biosystems) following the manufacturer's protocol. Out of 77 primary lung tumors tested, 54 were found to have increased copy number for Galanin.

Total RNA was isolated from human lung tumors and lung tumor cell lines using Trizol Reagent (Invitrogen) and treated with DNAase (Ambion) to eliminate genomic DNA. The reverse transcriptase reaction (at 48° C. for 30 min) was coupled with quantitative PCR measurement of cDNA copy number in a one-tube format according to the manufacturer (Perkin Elmer/Applied Biosystems). Galanin expression level in the samples was normalized using human β-actin and overexpression fold was calculated by comparing Galanin expression in tumor v. normal samples. The nucleotide sequences of Galanin are used to design suitable Taqman probes based on the sequences listed under the GenBank Accession Number A28025. The measurements of the mRNA level of each primary tumor and tumor cell line sample were normalized to the mRNA levels in normal lung tissue and normal human bronchial epithelial (NHBE) cell line, respectively. Relative numerical values of the mRNA levels are shown in Table 7. Of the 10 lung tumors studied shown in Table 5, all overexpress GALR3 and GALR2 at varying degree, including the sample LU44 which lacks GALR3 gene amplification. Of the 50 samples tested (see Table 7), 39 overexpress Galanin. The observation of Galanin message overexpression without concomitant gene amplification is reminiscent of a hallmark of oncogenes such as c-myc (Yoshimoto et al. Jpn. J. Cancer Res. 77, 540-545 (1986)) and AIB1 (Anzick et al. Science 277, 965-968 (1997)).

TABLE 7
Amplification and overexpression of the Galanin gene in lung tumors.

| | |
|---|---|
| Lung Tumor | Galanin |

| Designation | *DNA Copy Number | *RNA Expression |
|---|---|---|
| LU1 | 5.5 | Low RNA |
| LU2 | 3.1 | 96 |
| LU3 | 2.8 | Low RNA |
| LU4 | 5.4 | 251 |
| LU5 | 5.2 | 79 |
| LU6 | 7.1 | 17 |
| LU7 | 5.4 | 4.4 |
| LU8 | 4.2 | 67 |
| LU9 | 4.9 | 5.5 |
| LU10 | 1.4 | 7.3 |
| LU11 | 5.1 | 2.8 |
| LU12 | 3.4 | |
| LU13 | 3.3 | 1138 |
| LU14 | 2.6 | 11 |
| LU15 | 7.8 | Low RNA |
| LU16 | 2.9 | 14 |
| LU17 | 21 | 281 |
| LU18 | 6 | 32 |
| LU19 | 12 | |
| LU20 | 4.9 | 45 (30) |
| LU21 | 7.2 | 16 |
| LU22 | 3.3 | 6.7 |
| LU23 | 4.7 | Low RNA |
| LU24 | 16 | |
| LU25 | 6.6 | 3.7 |
| LU26 | | 90 |
| LU27 | 6.2 | |
| LU28 | 5.4 | 74 |
| LU29 | 3.6 | 2.8 |
| LU30 | 14 | 12 |
| LU31 | | 16 |
| LU32 | | |
| LU33 | | 7.2 |
| LU34 | 6.3 | 33 |
| LU35 | | |
| LU36 | 1.7 | 7.7 |
| LU37 | 13 | 17 |
| LU38 | 6.6 | Low RNA |
| LU39 | | Low RNA |
| LU41 | | 2.9 |
| LU42 | 5.6 | 7.4 |
| LU43 | 3.4 | 124 |
| LU44 | 2.7 | 17 |
| LU45 | 3.4 | |
| LU46 | 5.1 | 9.9 |
| LU47 | 3.8 | 4.2 |
| LU48 | 6.2 | 139 |
| LU49 | 6.3 | 18 |
| LU50 | 9.2 | |
| LU51 | 8.5 | 20 |
| LU52 | 8 | 75 |
| LU53 | 14 | |
| LU54 | 5 | 91 |
| LU55 | 11 | |
| LU56 | 9.1 | |
| LU57 | 6.6 | |
| LU58 | 5.3 | 35 |
| LU59 | 48 | 271 |
| LU60 | 11 | |
| LU61 | 8.2 | |
| LU62 | 26 | |
| LU63 | 21 | |
| LU64 | 17 | |
| LU65 | 11 | |
| LU66 | 17 | |
| LU67 | 8.4 | |
| LU68 | 9.1 | |
| LU69 | 3.6 | |
| LU70 | 2.4 | 13 |
| LU71 | 11 | |
| LU72 | 3.5 | Low RNA |
| LU73 | 18 | |
| LU74 | 18 | 3.2 |
| LU75 | 8.3 | 8.7 |
| LU76 | 7.9 | 2.2 |
| LU77 | 3.9 | 0.047 |
| LU78 | 3.2 | |
| LU79 | 7.6 | 1.6 |
| LU80 | 5.4 | |
| LU81 | 5.9 | 54 |
| LU82 | 6.9 | 270 |
| LU83 | 17 | |
| LU84 | 9.3 | 66 |
| LU85 | 6.9 | 1.5 |

*Low RNA = RNA of low quality; Not all the lung tumors have both DNA copy number and RNA expression determined. *DNA copy number and mRNA expression level were determined using the quantitative PCR method. The mRNA expression level of galanin in normal human lung tissue was used to normalize the data shown under mRNA expression.

### Example V

### Animal Tumorigenicity Assay for the Galanin Gene

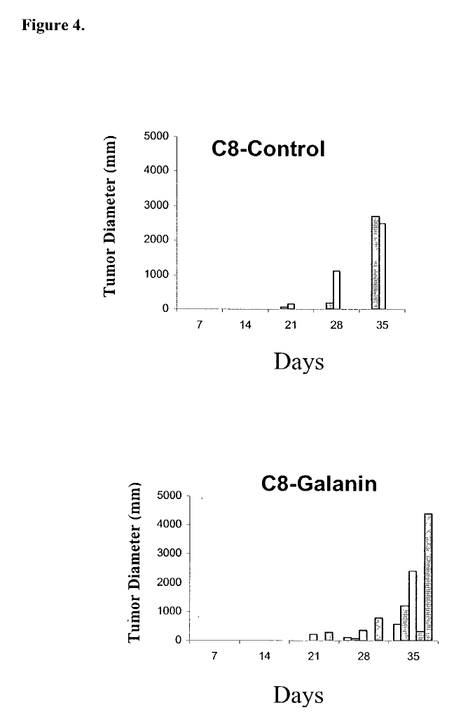

Galanin precursor or empty vector (pLPC) was transfected into rodent C8 cells via retrovirus (See FIG. 4). After drug selection, cells were collected and injected into 5 nude, athymic mice for tumor formation observation (250,000 cells/mouse). A clear enhancement of C8 cell-derived tumor growth was detected in galanin transfectants. At day 35 post injection, all 5 mice carrying galanin-C8 cells developed tumors (see FIG. 4B) whereas only 2 out of the 5 mice injected with the vector-C8 cells had tumors (see FIG. 4A). The results indicate that Galanin C8 Transfectants enhance tumorigenicity in animals. The results also provides that overexpression of galanin enhances tumorigenicity of host cells in animals, which indicates that galanin is an oncogene.

### Example VI

### Amplification and Overexpression of Galanin/GALR2/GALR3 Genes in Colon, Prostate, and Ovarian Tumors

Referring to Table 8, the DNA copy numbers were determined in the primary colon, prostate, and ovarian cancer samples using a Taqman 7700 and 7900 Sequence Detectors. The genomic DNAs were isolated from colon, prostate, and ovarian tumors. Primary colon, prostate, and ovarian tumor samples were subjected, along with Galanin or GALR2 or GALR3 Taqman probe set as described supra representing the target, and a reference probe representing a normal non-amplified region in the genome, to analysis by Taqman 7700 or 7900 Sequence Detectors following manufacturer's protocol. Increased copy number for Galanin, GALR2, and GALR3 genes were found in primary colon, prostate, and ovarian tumors tested (see Table 8).

Total RNA was isolated from human colon, prostate, and ovarian tumors using the Trizol reagent (Gibco). RNA of cultured normal human bronchial epithelial (NHBE) cell line (Clonetics) was prepared using the Trizol reagent. Reverse transcriptase (RT)-directed quantitative PCR was performed using the Taqman 7700 Sequence Detector (Applied Biosystems) to determine the Galanin, GALR2, and GALR3 mRNA levels in each sample. Human beta-actin mRNA was used as a control. The nucleotide sequences of Galanin, GALR2, and GALR3 were used to design suitable Taqman probes based on the sequences listed herein. The measurements of the mRNA level of each primary tumor and tumor cell line sample were normalized to the mRNA levels in normal colon, prostate, and ovarian tissues and NHBE, respectively. Overexpression of Galanin, GALR2, and GALR3 were detected in primary colon, prostate, and ovarian tumors tested (see Table 8).

TABLE 8
Galanin, GALR2 and GALR3 genes: amplification and overexpression in colon,
prostate, and ovarian tumors*.

| | | | | | Metastatic | | | |
|---|---|---|---|---|---|---|---|---|
| | Lung Tumor | | Colon Tumor | | Prostate Tumor | | Ovarian Tumor | |
| Gene | DNA | RNA | DNA | RNA | DNA | RNA | DNA | RNA |
| Galanin | 59% | 78% | 6% | 14% | 20% | 29% | 0% | 83% |
| | (45/77) | (39/50) | (1/16) | (1/7) | (4/15) | (2/7) | (0/16) | (15/18) |
| GALR2 | 21% | 40% | 0% | 9% | 0% | 42% | 6% | 6% |
| | (17/81) | (17/42) | (0/14) | (1/11) | (0/15) | (3/7) | (3/45) | (2/30) |
| GALR3 | 25% | 55% | 0% | 9% | 0% | 81% | 2% | 7% |
| | (21/83) | (21/38) | (0/14) | (1/11) | (0/16) | (9/11) | (1/48) | (2/27) |

*Numbers in parenthesis indicating positive samples per total number of samples tested for amplification (DNA) or overexpression (RNA) of Galanin, GALR2, or GALR3 gene.

### Example VII

### Fluorescence in situ Hybridization (FISH) Analysis of Human Lung Cancer Cell Line, EPLC-272H

FISH analysis of human lung cancer cell line, EPLC-272H, was performed using techniques well known in the art (see, for example, Heng et al., Cytogenetic Cell Genet, 93: 195, 2001; Angerer, Meth. Enzymol., 152: 649, 1987) to determine amplification of preprogalanin gene. Interphase chromosome slides were prepared by conventional method (see, for example, Heng and Tsui, Chromosoma 102:325, 1993). Multiple positive signals were detected with probe 2007L18, which is a BAC clone containing human preprogalanin gene. Neutral signals were seen with a control probe 282H10, which represents an unamplified region of human genome. FISH analysis of the cell line EPLC-272H indicated amplification of the preprogalanin gene.

### Example VIII

### Overexpression of Preprogalanin Enhances Tumorigenicity of EPLC-272H Cells in Nude Mice:

Retroviral transfectants of EPLC-272H cells overexpressing preprogalanin were created. Five million cells were implanted into each of 5 athymic nude mice subcutaneously. At day 14 post injection, all 5 mice carrying the preprogalanin gene developed tumors of varying sizes; one mouse died before reaching day 14. On the contrary, at day 14 no visible tumor was detected with the 5 mice injected with the control cells (vector-EPLC-272H) (see Table 9). The results indicate that preprogalanin-EPLC-272H transfectants enhance tumorigenicity in nude mice. The results also provides that overexpression of galanin enhances tumorigenicity of host cells in animals, which indicates that galanin is an oncogene.

TABLE 9
Overexpression of preprogalanin enhances tumorigenicity.

Days after injection

| | 7 days | 14 days |
|---|---|---|
| | Tumor size (mm³) | |
| vector-EPLC-272H | | |
| Mouse 1 | 0 | 0 |
| Mouse 2 | 0 | 0 |
| Mouse 3 | 0 | 0 |
| Mouse 4 | 0 | 0 |
| Mouse 5 | 0 | 0 |
| Preprogalanin-EPLC272H | | |
| Mouse 1 | 0 | 24 |
| Mouse 2 | 0 | Died |
| Mouse 3 | 0 | 40 |
| Mouse 4 | 0 | 180 |
| Mouse 5 | 0 | 32 |

### Example IX

### Small Interfering RNA (siRNA)

Sense and antisense siRNAs duplexes are made based upon targeted regions of a DNA sequence, as disclosed herein (for example, SEQ ID NO: 1, SEQ ID NO:3, SEQ ID NO:7, or a fragment thereof), are typically less than 100 base pairs (“bps”) in length and constituency and preferably are about 30 bps or shorter, and are made by approaches known in the art, including the use of complementary DNA strands or synthetic approaches. SiRNA derivatives employing polynucleic acid modification techniques, such as peptide nucleic acids, also can be employed according to the invention. The siRNAs are capable of causing interference and can cause post-transcriptional silencing of specific genes in cells, for example, mammalian cells (including human cells) and in the body, for example, mammalian bodies (including humans). Exemplary siRNAs according to the invention have up to 29 bps, 25 bps, 22 bps, 21 bps, 20 bps, 15 bps, 10 bps, 5 bps or any integer thereabout or therebetween.

A targeted region is selected from the DNA sequence (for example, SEQ ID NO:1, SEQ ID NO:3, SEQ ID NO:7, or a fragment thereof). Various strategies are followed in selecting target regions and designing siRNA oligos, for example, 5′ or 3′ UTRs and regions nearby the start codon should be avoided, as these may be richer in regulatory protein binding sites. Designed sequences preferably include AA-(N27 or less nucleotides)-TT and with about 30% to 70% G/C-content. If no suitable sequences are found, the fragment size is extended to sequences AA(N29 nucleotides). The sequence of the sense siRNA corresponds to, for example, (N27 nucleotides)-TT or N29 nucleotides, respectively. In the latter case, the 3′ end of the sense siRNA is converted to TT. The rationale for this sequence conversion is to generate a symmetric duplex with respect to the sequence composition of the sense and antisense 3′ overhangs. It is believed that symmetric 3′ overhangs help to ensure that the small interfering ribonucleoprotein particles (siRNPs) are formed with approximately equal ratios of sense and antisense target RNA-cleaving siRNPs (Elbashir et al. Genes & Dev. 15:188-200, 2001).

GALR3 siRNA: Sense or antisense siRNAs are synthesized based upon targeted regions of a DNA sequence, as disclosed herein (see SEQ ID NO: 1), include fragments having up to 29 bps, 25 bps, 22 bps, 21 bps, 20 bps, 15 bps, 10 bps, 5 bps or any integer thereabout or therebetween. For example, 29 bps siRNA include:

Targeted region (base position numbers 2-30, SEQ ID NO: 10) 5′-TGGCTGATGCCCAGAACATTTCACTGGAC-3′, and the corresponding sense siRNA (SEQ ID NO: 11) 5′-UGGCUGAUGCCCAGAACAUUUCACUGGAC-3′;

Targeted region (base position numbers 3-31, SEQ ID NO: 12) 5′-GGCTGATGCCCAGAACATTTCACTGGACA-3′, and the corresponding sense siRNA (SEQ ID NO: 13) 5′-GGCUGAUGCCCAGAACAUUUCACUGGACA-3′;

Targeted region (base position numbers 4-32, SEQ ID NO: 14) 5′-GCTGATGCCCAGAACATTTCACTGGACAG-3′, and the corresponding sense siRNA (SEQ ID NO: 15) 5′-GCUGAUGCCCAGAACAUUUCACUGGACAG-3′; and continuing in this progression to the end of GALR3 coding sequence, for example,

Targeted region (base position numbers 1071-1099, SEQ ID NO: 16) 5′-CGTCCACGGCGGAGAGGCTGCCCGAGGAC-3′, and the corresponding sense siRNA (SEQ ID NO: 17) 5′-CGUCCACGGCGGAGAGGCUGCCCGAGGAC-3′; and so on as set forth herein.

GALR2 siRNA: Sense or antisense siRNAs are synthesized based upon targeted regions of a DNA sequence, as disclosed herein (see SEQ ID NO:3), include fragments having up to 29 bps, 25 bps, 22 bps, 21 bps, 20 bps, 15 bps, 10 bps, 5 bps or any integer thereabout or therebetween. For example, 29.bps siRNA include:

Targeted region (base position numbers 2-30, SEQ ID NO: 18) 5′-TGAACGTCTCGGGCTGCCCAGGGGCCGGG-3′, and the corresponding sense siRNA (SEQ ID NO: 19) 5′-UGAACGUCUCGGGCUGCCCAGGGGCCGGG-3′;

Targeted region (base position numbers 3-31, SEQ ID NO:20) 5′-GAACGTCTCGGGCTGCCCAGGGGCCGGGA-3′, and the corresponding sense siRNA (SEQ ID NO:21) 5′-GAACGUCUCGGGCUGCCCAGGGGCCGGGA-3′;

Targeted region (base position numbers 4-32, SEQ ID NO:22) 5′-AACGTCTCGGGCTGCCCAGGGGCCGGGAA-3′, and the corresponding sense siRNA (SEQ ID NO:23) 5′-AACGUCUCGGGCUGCCCAGGGGCCGGGAA-3′; and continuing in this progression to the end of GALR2 coding sequence, for example,

Targeted region (base position numbers 1132-1160, SEQ ID NO:24) 5′-GGCGACAGCATCCTGACGGTTGATGTGGC-3′, and the corresponding sense siRNA (SEQ ID NO:25) 5′-GGCGACAGCAUCCUGACGGUUGAUGUGGC-3′; and so on as set forth herein.

Galanin siRNA: Sense or antisense siRNAs are synthesized based upon targeted 15 regions of a DNA sequence, as disclosed herein (see SEQ ID NO:7), include fragments having up to 29 bps, 25 bps, 22 bps, 21 bps, 20 bps, 15 bps, 10 bps, 5 bps or any integer thereabout or therebetween. For example, 29 bps siRNA include:

Targeted region (base position numbers 15-43, SEQ ID NO:26) 5′-TGGCCCGAGGCAGCGCCCTCCTGCTCGCC-3′, and the corresponding sense siRNA (SEQ ID NO:27) 5′-UGGCCCGAGGCAGCGCCCUCCUGCUCGCC-3′;

Targeted region (base position numbers 16-44, SEQ ID NO:28) 5′-GGCCCGAGGCAGCGCCCTCCTGCTCGCCT-3′, and the corresponding sense siRNA (SEQ ID NO:29) 5′-GGCCCGAGGCAGCGCCCUCCUGCUCGCCU-3′;

Targeted region (base position numbers 17-45, SEQ ID NO:30) 5′-GCCCGAGGCAGCGCCCTCCTGCTCGCCTC-3′, and the corresponding sense siRNA (SEQ ID NO:31) 5′-GCCCGAGGCAGCGCCCUCCUGCUCGCCUC-3′; and continuing in this progression to the end of Galanin coding sequence, for example,

Targeted region (base position numbers 353-381, SEQ ID NO:32) 5′-GCAGCCTCCTCAGAAGACATCGAGCGGTC-3′, and the corresponding sense siRNA (SEQ ID NO:33) 5′-GCAGCCUCCUCAGAAGACAUCGAGCGGUC-3′; and so on as set forth herein.

As described herein for GALR3, GALR2 and Galanin oligos also can be designed based on a set criteria. A 29 bps ‘sense’ sequences (for example, a target region starting at base position number 2 of the GALR3-coding sequence) containing a ‘C’ at the 3′ end can be selected from the GALR3-coding sequence (SEQ ID NO:1). A termination sequence (for example, AAAAAA, SEQ ID NO:34), an GALR3 antisense sequence, a loop (for example, GAAGCTTG, SEQ ID NO:35), and a reverse primer (for example, U6 reverse primer, GGTGTTTCGTCCTTTCCACAA, SEQ ID NO:36) can be subsequently added to the 29 bps sense strands to construct GALR3 PCR primers (see, for example, Paddison et al., Genes & Dev. 16: 948-958, 2002). Of course, other sense and anti-sense sequences can be selected from a target molecule to develop siRNAs for that molecule.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

We claim:

1. A method for diagnosing a cancer in a mammal, comprising:
a) determining Galanin gene copy number in a biological subject from a region of the mammal that is suspected to be precancerous or cancerous, thereby generating data for a test gene copy number; and
b) comparing the test gene copy number to data for a control gene copy number, wherein an amplification of the gene in the biological subject relative to the control indicates the presence of a precancerous lesion or a cancer in the mammal.

2. The method according to claim 1, wherein the control gene copy number is two copies per cell.

3. The method according to claim 1, wherein the data is stored in an electronic format.

4. The method according to claim 3, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

5. The method according to claim 3, wherein the data is displayed, transmitted or analyzed via electronic transmission, video display, or telecommunication.

6. A method for inhibiting cancer or precancerous growth in a mammalian tissue, comprising contacting the tissue with an inhibitor that interacts with Galanin DNA or RNA and thereby inhibits Galanin gene function.

7. The method according to claim 6, wherein the inhibitor is a siRNA, an antisense RNA, an antisense DNA, a decoy molecule, or a decoy DNA.

8. The method according to claim 6, wherein the inhibitor contains nucleotides, and wherein the inhibitor comprises less than about 100 bps in length.

9. The method according to claim 6, wherein the inhibitor is a ribozyme.

10. The method according to claim 6, wherein the inhibitor is a small molecule.

11. A method for inhibiting cancer or precancerous growth in a mammalian tissue, comprising contacting the tissue with an inhibitor of Galanin protein.

12. The method according to claim 11, wherein the inhibitor is an antibody that binds to Galanin protein.

13. The method according to claim 11, wherein the inhibitor is a small molecule that binds to Galanin protein.

14. The method according to claim 11, wherein the inhibitor is an antibody that blocks oncogenic function or anti-apoptotic activity of Galanin.

15. The method according to claim 11, wherein the inhibitor is an antibody that binds to a cell overexpressing Galanin protein.

16. An isolated Galanin gene amplicon, wherein the amplicon comprises more than one copy of a polynucleotide selected from the group consisting of:
a) a polynucleotide encoding the polypeptide set forth in SEQ ID NO:8 or SEQ ID NO:9;
b) a polynucleotide set forth in SEQ ID NO:7; and
c) a polynucleotide having at least about 90% sequence identity to the polynucleotide of a) or b).

17. A method for diagnosing a cancer in a mammal, comprising:
a) determining the level of Galanin in a biological subject from a region of the mammal that is suspected to be precancerous or cancerous, thereby generating data for a test level; and
b) comparing the test level to data for a control level, wherein an elevated test level of the biological subject relative to the control level indicates the presence of a precancerous lesion or a cancer in the mammal.

18. The method according to claim 17, wherein the control level is obtained from a database of Galanin levels detected in a normal biological subject.

19. The method according to claim 18, wherein the database contains control levels obtained from a demographically diverse population.

20. The method according to claim 17, wherein the data is stored in an electronic format.

21. The method according to claim 20, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

22. The method according to claim 20, wherein the data is displayed, transmitted or analyzed via electronic transmission, video display, or telecommunication.

23. A method of administering siRNA to a patient in need thereof, wherein the siRNA molecule is delivered in the form of a naked oligonucleotide or a vector, wherein the siRNA interacts with Galanin gene or Galanin transcript.

24. The method of claim 23, wherein the siRNA is delivered as a vector, wherein the vector is a plasmid, cosmid, bacteriophage, or a virus.

25. The method of claim 23, wherein the vector is a retrovirus or an adenovirus based vector.

26. A method of blocking in vivo expression of a gene by administering a vector encoding Galanin siRNA.

27. The method of claim 26, wherein the siRNA interferes with Galanin activity.

28. The method of claim 26, wherein the siRNA causes post-transcriptional silencing of Galanin gene in a mammalian cell.

29. The method of claim 28, wherein the cell is a human cell.

30. A method of screening a test molecule for Galanin antagonist activity comprising the steps of:
a) contacting the molecule with a cancer cell;
b) determining the level of Galanin in the cell, thereby generating data for a test level; and
c) comparing the test level to a control level, wherein a decrease in Galanin level in the cell relative to the control indicates Galanin antagonist activity of the test molecule.

31. The method of claim 30, wherein the level of Galanin is determined by reverse transcription and polymerase chain reaction (RT-PCR).

32. The method of claim 30, wherein the level of Galanin is determined by Northern hybridization.

33. The method of claim 30, wherein the test molecule is an antibody, a nucleotide, or a small molecule.

34. The method of claim 30, wherein the cell is obtained from a lung cancer, a brain cancer, a breast cancer, a colon cancer, a prostate cancer, or an ovarian cancer.

35. A method of screening a test molecule for Galanin antagonist activity comprising the steps of:
a) contacting the molecule with Galanin; and
b) determining the effect of the test molecule on Galanin.

36. The method according to claim 35, wherein the effect is determined via a binding assay.

37. A method for diagnosing a cancer in a mammal, comprising:
a) determining GALR2 gene copy number in a biological subject from a region of the mammal that is suspected to be precancerous or cancerous, thereby generating data for a test gene copy number; and
b) comparing the test gene copy number to data for a control gene copy number, wherein an amplification of the gene in the biological subject relative to the control indicates the presence of a precancerous lesion or a cancer in the mammal.

38. The method according to claim 37, wherein the control gene copy number is two copies per cell.

39. The method according to claim 37, wherein the data is stored in an electronic format.

40. The method according to claim 39, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

41. The method according to claim 39, wherein the data is displayed, transmitted or analyzed via electronic transmission, video display, or telecommunication.

42. A method for inhibiting cancer or precancerous growth in a mammalian tissue, comprising contacting the tissue with an inhibitor that interacts with GALR2 DNA or RNA and thereby inhibits GALR2 gene function.

43. The method according to claim 42, wherein the inhibitor is a siRNA, an antisense RNA, an antisense DNA, a decoy molecule, or a decoy DNA.

44. The method according to claim 42, wherein the inhibitor contains nucleotides, and wherein the inhibitor comprises less than about 100 bps in length.

45. The method according to claim 42, wherein the inhibitor is a ribozyme.

46. The method according to claim 42, wherein the inhibitor is a small molecule.

47. A method for inhibiting cancer or precancerous growth in a mammalian tissue, comprising contacting the tissue with an inhibitor of GALR2 protein.

48. The method according to claim 47, wherein the inhibitor is an antibody that binds to GALR2 protein.

49. The method according to claim 47, wherein the inhibitor is a small molecule that binds to GALR2 protein.

50. The method according to claim 47, wherein the inhibitor is an antibody that blocks oncogenic function or anti-apoptotic activity of GALR2.

51. The method according to claim 47, wherein the inhibitor is an antibody that binds to a cell overexpressing GALR2 protein.

52. An isolated GALR2 gene amplicon, wherein the amplicon comprises more than one copy of a polynucleotide selected from the group consisting of:
a) a polynucleotide encoding the polypeptide set forth in SEQ ID NO:4;
b) a polynucleotide set forth in SEQ ID NO:3; and
c) a polynucleotide having at least about 90% sequence identity to the polynucleotide of a) or b).

53. A method for diagnosing a cancer in a mammal, comprising:
a) determining the level of GALR2 in a biological subject from a region of the mammal that is suspected to be precancerous or cancerous, thereby generating data for a test level; and
b) comparing the test level to data for a control level, wherein an elevated test level of the biological subject relative to the control level indicates the presence of a precancerous lesion or a cancer in the mammal.

54. The method according to claim 53, wherein the control level is obtained from a database of GALR2 levels detected in a normal biological subject.

55. The method according to claim 54, wherein the database contains control levels obtained from a demographically diverse population.

56. The method according to claim 53, wherein the data is stored in an electronic format.

57. The method according to claim 56, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

58. The method according to claim 56, wherein the data is displayed, transmitted or analyzed via electronic transmission, video display, or telecommunication.

59. A method of administering siRNA to a patient in need thereof, wherein the siRNA molecule is delivered in the form of a naked oligonucleotide or a vector, wherein the siRNA interacts with GALR2 gene or GALR2 transcript.

60. The method of claim 59, wherein the siRNA is delivered as a vector, wherein the vector is a plasmid, cosmid, bacteriophage, or a virus.

61. The method of claim 59, wherein the vector is a retrovirus or an adenovirus based vector.

62. A method of blocking in vivo expression of a gene by administering a vector encoding GALR2 siRNA.

63. The method of claim 62, wherein the siRNA interferes with GALR2 activity.

64. The method of claim 62, wherein the siRNA causes post-transcriptional silencing of GALR2 gene in a mammalian cell.

65. The method of claim 64, wherein the cell is a human cell.

66. A method of screening a test molecule for GALR2 antagonist activity comprising the steps of:
a) contacting the molecule with a cancer cell;
b) determining the level of GALR2 in the cell, thereby generating data for a test level; and
c) comparing the test level to a control level, wherein a decrease in GALR2 level in the cell relative to the control indicates GALR2 antagonist activity of the test molecule.

67. The method of claim 66, wherein the level of GALR2 is determined by reverse transcription and polymerase chain reaction (RT-PCR).

68. The method of claim 66, wherein the level of GALR2 is determined by Northern hybridization.

69. The method of claim 66, wherein the test molecule is an antibody, a nucleotide, or a small molecule.

70. The method of claim 66, wherein the cell is obtained from a lung cancer, a brain cancer, a breast cancer, a colon cancer, a prostate cancer, or an ovarian cancer.

71. A method of screening a test molecule for GALR2 antagonist activity comprising the steps of:
a) contacting the molecule with GALR2; and
b) determining the effect of the test molecule on GALR2.

72. The method according to claim 71, wherein the effect is determined via a binding assay.

73. A method for diagnosing a cancer in a mammal, comprising:
a) determining GALR3 gene copy number in a biological subject from a region of the mammal that is suspected to be precancerous or cancerous, thereby generating data for a test gene copy number; and
b) comparing the test gene copy number to data for a control gene copy number, wherein an amplification of the gene in the biological subject relative to the control indicates the presence of a precancerous lesion or a cancer in the mammal.

74. The method according to claim 73, wherein the control gene copy number is two copies per cell.

75. The method according to claim 73, wherein the data is stored in an electronic format.

76. The method according to claim 75, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

77. The method according to claim 75, wherein the data is displayed, transmitted or analyzed via electronic transmission, video display, or telecommunication.

78. A method for inhibiting cancer or precancerous growth in a mammalian tissue, comprising contacting the tissue with an inhibitor that interacts with GALR3 DNA or RNA and thereby inhibits GALR3 gene function.

79. The method according to claim 78, wherein the inhibitor is a siRNA, an antisense RNA, an antisense DNA, a decoy molecule, or a decoy DNA.

80. The method according to claim 78, wherein the inhibitor contains nucleotides, and wherein the inhibitor comprises less than about 100 bps in length.

81. The method according to claim 78, wherein the inhibitor is a ribozyme.

82. The method according to claim 78, wherein the inhibitor is a small molecule.

83. A method for inhibiting cancer or precancerous growth in a mammalian tissue, comprising contacting the tissue with an inhibitor of GALR3 protein.

84. The method according to claim 83, wherein the inhibitor is an antibody that binds to GALR3 protein.

85. The method according to claim 83, wherein the inhibitor is a small molecule that binds to GALR3 protein.

86. The method according to claim 83, wherein the inhibitor is an antibody that blocks oncogenic function or anti-apoptotic activity of GALR3.

87. The method according to claim 83, wherein the inhibitor is an antibody that binds to a cell overexpressing GALR3 protein.

88. An isolated GALR3 gene amplicon, wherein the amplicon comprises more than one copy of a polynucleotide selected from the group consisting of:
a) a polynucleotide encoding the polypeptide set forth in SEQ ID NO:2;
b) a polynucleotide set forth in SEQ ID NO: 1; and
c) a polynucleotide having at least about 90% sequence identity to the polynucleotide of a) or b).

89. A method for diagnosing a cancer in a mammal, comprising:
a) determining the level of GALR3 in a biological subject from a region of the mammal that is suspected to be precancerous or cancerous, thereby generating data for a test level; and
b) comparing the test level to data for a control level, wherein an elevated test level of the biological subject relative to the control level indicates the presence of a precancerous lesion or a cancer in the mammal.

90. The method according to claim 89, wherein the control level is obtained from a database of GALR3 levels detected in a normal biological subject.

91. The method according to claim 90, wherein the database contains control levels obtained from a demographically diverse population.

92. The method according to claim 89, wherein the data is stored in an electronic format.

93. The method according to claim 92, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

94. The method according to claim 92, wherein the data is displayed, transmitted or analyzed via electronic transmission, video display, or telecommunication.

95. A method of administering siRNA to a patient in need thereof, wherein the siRNA molecule is delivered in the form of a naked oligonucleotide or a vector, wherein the siRNA interacts with GALR3 gene or GALR3 transcript.

96. The method of claim 95, wherein the siRNA is delivered as a vector, wherein the vector is a plasmid, cosmid, bacteriophage, or a virus.

97. The method of claim 95, wherein the vector is a retrovirus or an adenovirus based vector.

98. A method of blocking in vivo expression of a gene by administering a vector encoding GALR3 siRNA.

99. The method of claim 98, wherein the siRNA interferes with GALR3 activity.

100. The method of claim 98, wherein the siRNA causes post-transcriptional silencing of GALR3 gene in a mammalian cell.

101. The method of claim 100, wherein the cell is a human cell.

102. A method of screening a test molecule for GALR3 antagonist activity comprising the steps of:
a) contacting the molecule with a cancer cell;
b) determining the level of GALR3 in the cell, thereby generating data for a test level; and
c) comparing the test level to a control level, wherein a decrease in GALR3 level in the cell relative to the control indicates GALR3 antagonist activity of the test molecule.

103. The method of claim 102, wherein the level of GALR3 is determined by reverse transcription and polymerase chain reaction (RT-PCR).

104. The method of claim 102, wherein the level of GALR3 is determined by Northern hybridization.

105. The method of claim 102, wherein the test molecule is an antibody, a nucleotide, or a small molecule.

106. The method of claim 102, wherein the cell is obtained from a lung cancer, a brain cancer, a breast cancer, a colon cancer, a prostate cancer, or an ovarian cancer.

107. A method of screening a test molecule for GALR3 antagonist activity comprising the steps of:
a) contacting the molecule with GALR3; and
b) determining the effect of the test molecule on GALR3.

108. The method according to claim 107, wherein the effect is determined via a binding assay.

109. A method for inhibiting cancer or precancerous growth in a mammalian tissue, comprising contacting the tissue with an inhibitor that interacts with GALR2-GALR3 heterocomplex DNA or RNA and thereby inhibits GALR2-GALR3 heterocomplex gene function.

110. The method according to claim 109, wherein the inhibitor is a siRNA, an antisense RNA, an antisense DNA, a decoy molecule, or a decoy DNA.

111. The method according to claim 109, wherein the inhibitor contains nucleotides, and wherein the inhibitor comprises less than about 100 bps in length.

112. The method according to claim 109, wherein the inhibitor is a ribozyme.

113. The method according to claim 109, wherein the inhibitor is a small molecule.

114. A method for inhibiting cancer or precancerous growth in a mammalian tissue, comprising contacting the tissue with an inhibitor of GALR2-GALR3 heterocomplex protein.

115. The method according to claim 114, wherein the inhibitor is an antibody that binds to GALR2-GALR3 heterocomplex protein.

116. The method according to claim 114, wherein the inhibitor is a small molecule that binds to GALR2-GALR3 heterocomplex protein.

117. The method according to claim 114, wherein the inhibitor is an antibody that blocks oncogenic function or anti-apoptotic activity of GALR2-GALR3 heterocomplex.

118. The method according to claim 114, wherein the inhibitor is an antibody that binds to a cell overexpressing GALR2-GALR3 heterocomplex protein.

119. A method for diagnosing a cancer in a mammal, comprising:
a) determining the level of GALR2-GALR3 heterocomplex in a biological subject from a region of the mammal that is suspected to be precancerous or cancerous, thereby generating data for a test level; and
b) comparing the test level to data for a control level, wherein an elevated test level of the biological subject relative to the control level indicates the presence of a precancerous lesion or a cancer in the mammal.

120. The method according to claim 119, wherein the control level is obtained from a database of GALR2-GALR3 heterocomplex levels detected in a normal biological subject.

121. The method according to claim 120, wherein the database contains control levels obtained from a demographically diverse population.

122. The method according to claim 119, wherein the data is stored in an electronic format.

123. The method according to claim 122, wherein the electronic format is selected from the group consisting of electronic mail, disk, compact disk (CD), digital versatile disk (DVD), memory card, memory chip, ROM or RAM, magnetic optical disk, tape, video, video clip, microfilm, internet, shared network, and shared server.

124. The method according to claim 122, wherein the data is displayed, transmitted or analyzed via electronic transmission, video display, or telecommunication.

125. A method of screening a test molecule for GALR2-GALR3 heterocomplex antagonist activity comprising the steps of:
a) contacting the molecule with a cancer cell;
b) determining the level of GALR2-GALR3 heterocomplex in the cell, thereby generating data for a test level; and
c) comparing the test level to a control level, wherein a decrease in GALR2-GALR3 heterocomplex level in the cell relative to the control indicates GALR2-GALR3 heterocomplex antagonist activity of the test molecule.

126. The method of claim 125, wherein the level of GALR2-GALR3 heterocomplex is determined by Northern hybridization.

127. The method of claim 125, wherein the test molecule is an antibody, a nucleotide, or a small molecule.

128. The method of claim 125, wherein the cell is obtained from a lung cancer, a brain cancer, a breast cancer, a colon cancer, a prostate cancer, or an ovarian cancer.

129. A method of screening a test molecule for GALR2-GALR3 heterocomplex antagonist activity comprising the steps of:
a) contacting the molecule with GALR2-GALR3 heterocomplex; and
b) determining the effect of the test molecule on GALR2-GALR3 heterocomplex.

130. The method according to claim 129, wherein the effect is determined via a binding assay.
